SEQUENCE LISTING (5 sequences)

SEQ ID NO: 1 - DNA, 12 bp, Artificial
  Human
gcgtaggaggca

SEQ ID NO: 2 - DNA, 14 bp, Artificial
  Human
gaggaggaggagga

SEQ ID NO: 3 - DNA, 16 bp, Artificial
  Mouse
aagtgagtgggtgttt

SEQ ID NO: 4 - DNA, 18 bp, Artificial
  Sequencing primer
tgtaaaacgacggccagt

SEQ ID NO: 5 - DNA, 18 bp, Artificial
  Sequencing primer
caggaaacagctatgacc

(19) United States
(12) Patent Application Publication
(10) Pub. No.: US 2005/0158733 A1
(43) Pub. Date: Jul. 21, 2005

(54) EGR genes as targets for the diagnosis and treatment of schizophrenia

(76) Inventors: David J. Gerber, Somerville, MA (US); Joseph A. Gogos, Riverdale, NY (US); Diana Hall, Lausanne (CH); Maria Karayiorgou, Riverdale, NY (US); Susumu Tonegawa, Chestnut Hill, MA (US)

Correspondence Address: CHOATE, HALL & STEWART LLP, EXCHANGE PLACE, 53 STATE STREET, BOSTON, MA, 02109, US

(21) Appl. No.: 10/881,185

(22) Filed: Jun. 30, 2004

Related U.S. Application Data
(60) Provisional application No. 60/484,043, filed on Jun. 30, 2003.

(51) Int. Cl. C12Q 1/68

(52) U.S. Cl. 435/6

(57) ABSTRACT

The present invention provides targets, methods, and reagents for the diagnosis and treatment of schizophrenia and related conditions. The invention provides methods for the diagnosis of schizophrenia and susceptibility to schizophrenia by detection of polymorphisms, mutations, variations, alterations in expression, etc., in genes encoding an EGR molecule or an EGR interacting molecule, or polymorphisms linked to such genes. The invention provides oligonucleotides, arrays, and antibodies for detection of polymorphisms and variants. The invention provides transgenic mice having alterations in such genes. The invention also provides methods of treating schizophrenia by administering compounds that target these genes. The invention further provides screening methods for identifying such compounds and compounds obtained by perfoming the screens.

### CROSS-REFERENCE TO RELATED APPLICATIONS

This application claims priority to U.S. Provisional Application No. 60/484,043, filed Jun. 30, 2003, which is herein incorporated by reference.

### GOVERNMENT SUPPORT

The United States Government has provided grant support utilized in the development of the present invention. In particular, P50-MH58880, awarded by the National Institute of Health, and R01-MH61399, awarded by the National Institute of Health have supported development of this invention. The United States Government may have certain rights in the invention.

### BACKGROUND

Schizophrenia is a severe psychiatric condition that affects approximately one percent of the population worldwide (Lewis, D. A. & Lieberman, J. A. (2000) Neuron 28, 325-3). The disease is characterized by a variety of so-called “positive” symptoms that tend to occur episodically, including hallucinations, delusions, paranoia, and psychosis and/or relatively persistent symptoms such as flattened affect, social withdrawal, impaired attention, and cognitive impairments. Symptoms in the latter category are frequently referred to as “negative symptoms”.

Studies of the inheritance of schizophrenia have revealed that it is a multi-factorial disease characterized by multiple genetic susceptibility elements, each likely contributing a modest increase in risk (Karayiorgou, M. & Gogos, J. A. (1997) Neuron 19, 967-79). Family linkage studies and studies of chromosomal abnormalities associated with schizophrenia have identified a number of schizophrenia susceptibility loci (Karayiorgou, M. & Gogos, J. A. (1997) Neuron 19, 967-79; Thaker, G. K. & Carpenter, W. T., Jr. (2001) Nat Med 7, 667-71). These loci encompass relatively large chromosomal regions and can contain hundreds of genes. Therefore, the identification of specific susceptibility genes in these regions is challenging.

In addition to direct genetic analysis, a longstanding body of pharmacological studies has led to the prevailing hypotheses that dysfunction of dopaminergic or NMDA receptor-mediated signaling are major contributing factors in schizophrenia pathogenesis (Seeman, P. (1987) Synapse 1, 133-52; Carlsson, A., et al., (2001) Annu Rev Pharmacol Toxicol 41, 237-60). The dopamine hypothesis for the pathophysiology of schizophrenia maintains that dysfunction of the dopamine neurotransmitter system plays a key role in the abnormalities that occur in schizophrenia. This hypothesis stems from the observation that many drugs effective in treating schizophrenia share the common property of blocking dopamine receptors. In addition, certain of the symptoms of schizophrenia can be reproduced by drugs such as amphetamine that act positively on the dopaminergic system. The glutamate dysfunction hypothesis provides an alternate, and not necessarily inconsistent potential explanation for the etiology of schizophrenia. This hypothesis arose from the observation that exposure to certain compounds such as phencyclidine (PCP) and MK-801, which act as antagonists of NMDA receptors (physiological receptors for glutamate), leads to development of schizophrenia-like symptoms. (See, e.g., Javitt, D. and Zukin, R., Am J Psychiatry, 1991 October; 148 (10): 1301-8. Despite the appeal of these hypotheses, convincing direct genetic, physiological, or biochemical evidence for association of dopamine receptors or NMDA receptors with schizophrenia has not been obtained. In addition, although various pharmacological agents for the treatment of schizophrenia exist and are widely used, no truly satisfactory therapy exists.

Thus there remains a need in the art for improved understanding of the etiology of schizophrenia. In addition, there remains a need in the art for improved methods and reagents for the diagnosis and treatment of schizophrenia and susceptibility to schizophrenia.

### SUMMARY OF THE INVENTION

The present invention relates to the identification of expression products of early growth response (EGR) genes and of genes encoding EGR interacting molecules as targets for the diagnosis and treatment of schizophrenia and related conditions. The invention provides methods for diagnosis of schizophenia and treatment of schizophrenia by detecting a polymorphic variant of a polymorphism in a coding or noncoding portion of a gene encoding an EGR molecule or encoding an EGR interacting molecule, or detecting a polymorphic variant of a polymorphism in a genomic region linked to such a gene, in a sample obtained or derived from a subject. The invention further provides methods for prevention and/or treatment of schizophrenia by modulating the expression level and/or activity of an EGR molecule or of an endogenous target or regulator of an EGR molecule. The invention further provides methods for identifying reagents useful for the diagnosis of schizophrenia and/or related conditions. In addition, the invention provides methods for identifying compounds useful for the prevention and/or treatment of schizophrenia and/or related disorders.

In particular, the invention provides a variety of methods for diagnosis of schizophrenia or related conditions. For example, the invention provides a method for the diagnosis of schizophrenia or schizophrenia susceptibility comprising: (i) providing a sample obtained from a subject to be tested for schizophrenia or schizophrenia susceptibility; and (ii) detecting a polymorphic variant of a polymorphism in a coding or noncoding portion of a gene encoding an EGR molecule or encoding an EGR interacting molecule, or detecting a polymorphic variant of a polymorphism in a genomic region linked to such a gene, in the sample in the sample. The invention further provides a method for the diagnosis of schizophrenia or schizophrenia susceptibility comprising: (i) providing a sample obtained from a subject to be tested for schizophrenia or schizophrenia susceptibility; and (ii) detecting an alteration or variation in expression or activity of an EGR molecule or an EGR interacting molecule in the sample, relative to the expression or activity of the EGR molecule or EGR interacting molecule that would be expected in a sample obtained from a normal subject.

In another aspect, the invention provides methods for the detection of polymorphisms, mutations, variations, alterations in expression, etc., in such genes and/or in their mRNA or protein expression products for use in the diagnosis of schizophrenia or related conditions or susceptibility to schizophrenia or related conditions. Such methods are useful for various purposes, including diagnosis.

According to another aspect, the invention provides a number of different in vitro and in vivo methods of screening for compounds useful in treating schizophrenia and/or related conditions including methods of screening for compounds in various animal models.

In another aspect, the invention provides compounds identified according to these screening methods, and pharmaceutical compositions including these compounds.

In another aspect, the invention provides a variety of methods of treating schizophrenia or susceptibility to schizophrenia. For example, the invention provides a method for treating schizophrenia or susceptibility to schizophrenia comprising: (i) providing a subject at risk of or suffering from schizophrenia; and (ii) administering a compound that modulates activity or abundance of an EGR molecule or an EGR interacting molecule to the subject. In certain embodiments of the invention the compound disrupts binding between an EGR protein and an NAB protein. The invention further provides a method for treating schizophrenia or susceptibility to schizophrenia comprising: (i) providing a subject at risk of or suffering from schizophrenia; and (ii) administering a compound that modulates intracellular calcium levels to the subject. The compounds for use in the various treatment methods described herein may be identified according to any of the inventive screens described herein, or using other approaches.

The invention further provides reagents such as oligonucleotides, oligonucleotide arrays, antibodies, and transgenic mice, including knockout and knockdown mice, and methods for their use in performing screens for compounds useful in treating schizophrenia or related conditions.

The contents of all patents, patent applications, journal articles, books, and other references cited are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of March 2002; Sambrook, Russell, Sambrook, Molecular Cloning: A Laboratory Manual, 3^{rd} ed., Cold Spring Harbor Laboratory Press, Cold Spring Harbor, 2001; and Using Antibodies: A Laboratory Manual, Harlow, E. and Lane, D. (Editors) New York: Cold Spring Harbor Laboratory Press, 1998; Goodman and Gilman's The Pharmacological Basis of Therapeutics, 10^{th} Ed. McGraw Hill, 2001; Katzung, B. (ed.) Basic and Clinical Pharmacology, McGraw-Hill/Appleton & Lange; 8th edition (Sep. 21, 2000); and Kandel, E., Schwartz, J. H., Jessell, T. M., (eds.), Principles of Neural Science, 4^{th} ed., McGraw Hill, 2000. If there is a conflict between any of the incorporated references and the instant specification, the specification shall control.

### BRIEF DESCRIPTION OF THE DRAWING

FIG. 1. Genomic position of EGR3 with respect to PPP3CC and markers from relevant linkage studies. PPP3CC is located at 8p21.3 at nucleotide position, 22651228-22751312, EGR3 is located at 8p21.3 at nucleotide position 22898401-22903983. D8S136 [Pulver et al., 1995 (2)] is located at nucleotide position 22785956; D8S1771 [Blouin et al., 1998 (4); Gurling et al., 2001 (5)] is located at nucleotide position, 25794644; D8S1752 [Blouin et al., 1998 (4)] is located at nucleotide position, 23022206; D8S1715, D8S133 [Kendler et al., 1996 (3)] are located at nucleotide positions 22321170 and 19849292, respectively. All nucleotide positions are from the November, 2002, human draft sequence.

FIG. 2. Genomic position of EGR1 with respect to markers from relevant linkage studies. EGR1 is located at 5q31.2 at nucleotides 137832044-137835867. D5S804 and D5S393 [Straub et al. (9)] are located at nucleotide positions 125216237 and 135732370, respectively. IL9 and D5S399 [Schwab et al. (10)] are located at nucleotide positions 135262379 and 135994380, respectively. D5S414 [Paunio et al. (11)] is located at nucleotide position 137709517 and CSF1R [Hovatta et al. (12)] is located at nucleotide position 149438979. All nucleotide positions are from the April, 2003, human draft sequence.

FIG. 3A (adapted from 30) is a schematic diagram showing an alignment of the four members of the EGR transcription factor family. The three zinc finger DNA binding domains are labeled and indicated in black. The R1 repression domain, which binds to the NAB (NGFI-A) binding proteins and is absent in EGR4 is indicated in gray.

FIG. 3B (adapted from 30) is a schematic diagram showing an EGR family member binding to a canonical EGR response element (RE) upstream of a hypothetical target gene.

FIG. 4 (adapted from 68) shows a schematic diagram of NAB1 indicating the positions of the two conserved NCD1 and NCD2 domains present within NAB1 and NAB2. NCD1 mediates binding to EGR family members containing the R1 domain. NCD2 is believed to repress EGR-mediated transactivation.

### DEFINITIONS

Agonist: The term agonist refers to a molecule that increases, or prolongs the duration of, the effect of a polypeptide or a nucleic acid. Agonists may include proteins, nucleic acids, carbohydrates, lipids, small molecules, ions, or any other molecules that modulate the effect of the polypeptide or nucleic acid. An agonist may be a direct agonist, in which case it is a molecule that exerts its effect by binding to the polypeptide or nucleic acid, or an indirect agonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Allele: The term allele refers to one of the different forms of a gene or DNA sequence that can exist at a single locus within the genome. The term includes both naturally occurring alleles, which are typically studied in genetic linkage and association studies, and genetically engineered alleles.

Antagonist: The term antagonist refers to a molecule that decreases, or reduces the duration of, the effect of a polypeptide or a nucleic acid. Antagonists may include proteins, nucleic acids, carbohydrates, or any other molecules that modulate the effect of the polypeptide or nucleic acid. An antagonist may be a direct antagonist, in which case it is a molecule that exerts its effect by binding to the polypeptide or nucleic acid, or an indirect antagonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Preferred antibodies, antibody fragments, and/or protein domains comprising an antigen binding site may be generated and/or selected in vitro, e.g., using techniques such as phage display (Winter, G. et al. 1994. Annu. Rev. Immunol. 12: 433-455, 1994), ribosome display (Hanes, J., and Pluckthun, A. Proc. Natl. Acad. Sci. USA. 94: 4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Diagnostic information: As used herein, diagnostic information or information for use in diagnosis is any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as schizophrenia), information related to the nature or classification of a disease, information related to prognosis and/or information useful in selecting an appropriate treatment.

Effective amount: In general, an “effective amount” of an active agent refers to an amount necessary to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, in the case of an agent for the treatment of schizophrenia, an effective amount may be an amount sufficient to result in clinical improvement of the patient, e.g., relief of one or more symptoms of schizophrenia, and/or improved results on a quantitative test of a phenotype suggestive of schizophrenia.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as microRNAs (mRNAs), tRNAs, etc. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification). A compound or agent is said to increase gene expression if application of the compound or agent to a cell or subject results in an increase in either an RNA or polypeptide expression product or both. A compound or agent is said to decrease gene expression if application of the compound or agent to a cell or subject results in a decrease in either an RNA or polypeptide expression product or both.

Homology: The term homology refers to a degree of similarity between two or more nucleic acid sequences or between two or more amino acid sequences. As is well known in the art, given any nucleotide or amino acid sequence, homologous sequences may be identified by searching databases (e.g., GenBank, EST [expressed sequence tag]databases, GST [gene sequence tag] databases, GSS [genome survey sequence] databases, organism sequencing project databases) using computer programs such as BLASTN for nucleotide sequences and BLASTP, gapped BLAST, and PSI-BLAST for amino acid sequences. These programs are described in Altschul, SF, et al., Basic local alignment search tool, J. Mol. Biol., 215 (3): 403-410, 1990, Altchul, SF and Gish, W, Methods in Enzymology, and Altschul, SF, et al., “Gapped BLAST and PSI-BLAST: a new generation of protein database search programs”, Nucleic Acids Res. 25: 3389-3402, 1997. In addition to identifying homologous sequences, the programs mentioned above typically provide an indication of the degree of identity homology, e.g., percent identity. The terms “identical” or percent “identity,” as applied to two or more nucleic acid or polypeptide sequences, refers to two or more sequences or subsequences that are the same or have a specified percentage of amino acid residues or nucleotides that are the same (e.g, about 50% identity, 55%, 60%, 65%, 70%, 75%, 80%, 85%, 90%, 91%, 92%, 93%, 94%, 95%, 96%, 97%, 98%, 99%), or higher identity over a portion of the sequence, (e.g., at least 50 residues, at least 100 residues, at least 10%, 20%, 30%, 40%, 50%, 60%, 70%, 80%, 90%, etc. of the sequence), or over the entire sequence, when compared and aligned for maximum correspondence over a comparison window or designated region). Percent identity can be measured using a sequence comparison methodology such as any of the programs referred to above, or subsequent versions, e.g., with default parameters described below, or manually. The alignment preferably considers gaps, e.g., it aligns particular regions of the sequence so as to achieve maximal alignment, inserting gaps where appropriate. Determining the degree of identity or homology that exists between two or more amino acid sequences or between two or more nucleotide sequences can also be conveniently performed using any of a variety of other algorithms and computer programs known in the art or manually. Discussion and sources of appropriate programs may be found, for example, in Baxevanis, A., and Ouellette, B. F. F., Bioinformatics: A Practical Guide to the Analysis of Genes and Proteins, Wiley, 1998; and Misener, S. and Krawetz, S. (eds.), Bioinformatics Methods and Protocols (Methods in Molecular Biology, Vol. 132), Humana Press, 1999.

Hybridize: The term “hybridize”, as used herein, refers to the interaction between two complementary nucleic acid sequences. The phrase “hybridizes under high stringency conditions” describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based various factors such as the length of the hybridizing sequences, whether they contain RNA or DNA, etc. For example, appropriate temperatures for high, medium, or low stringency hybridization will generally be lower for shorter sequences such as oligonucleotides than for longer sequences. Additional examples of hybridization conditions of varying stringency are found, for example, in Ausubel, et al., Current Protocols in Molecular Biology, John Wiley & Sons, N.Y., edition as of March 2002.

Isolated: As used herein, “isolated” means 1) separated from at least some of the components with which it is usually associated in nature; 2) prepared or purified by a process that involves the hand of man; and/or 3) not occurring in nature.

Linkage or linked: As used herein, linkage or linked generally refers to genetic linkage. Two loci (e.g., a DNA marker locus and a disease locus such as a mutation causing disease) are said to be genetically linked when the probability of a recombination event occurring between these two loci is below 50% (which equals the probability of recombination between two unlinked loci). The terms linkage or linked may also refer to physical linkage. In general, two loci are physically linked when they are present on the same contiguous piece of DNA. The greater the physical distance between the two loci, the less the degree of physical linkage. It will be appreciated that although there is a correspondence between genetic and physical linkage, the correspondence may be imprecise and can be nonlinear. For example, two loci that are separated by any particular number of bases may be closely linked genetically if the recombination frequency in the region between the loci is low, but may be essentially genetically unlinked or only weakly linked if the recombination frequency between the two loci is high.

Oligonucleotide: The term oligonucleotide refers to a nucleic acid (which can be DNA or RNA) ranging in length from 2 to approximately 70 bases. Oligonucleotides are often synthetic but can also be produced from naturally occurring polynucleotides. An oligonucleotide probe or primer is an oligonucleotide, typically single-stranded, that is capable of binding to a target nucleic acid of complementary sequence through one or more types of chemical bonds, usually through complementary pairing via hydrogen bond formation. Oligonucleotide probes and/or primers are often 5 to 60 bases and in specific embodiments may be between 10 and 40, or 15 and 30 bases long. An oligonucleotide probe or primer may include natural (e.g., A, G, C or T) or modified bases (7-deazaguanosine, inosine, etc.). In addition, the bases may be joined by a linkage other than a phosphodiester bond, such as a phosphoramidite linkage or a phosphorothioate linkage, or they may be peptide nucleic acids in which the constituent bases are joined by peptide bonds rather than by phosphodiester bonds, so long as such linkages do not interfere with hybridization. Any of the oligonucleotides described herein may be provided in isolated form or purified form.

Operably linked: The term operably linked, in reference to nucleic acids, refers to a relationship between two nucleic acid sequences wherein the expression or processing of one of the nucleic acid sequences is controlled by, regulated by, modulated by, etc. the other nucleic acid sequence. For example, a promoter is operably linked with a coding sequence if the promoter controls transcription of the coding sequence. Preferably a nucleic acid sequence that is operably linked to a second nucleic acid sequence is covalently linked, either directly or indirectly, to such a sequence, although any effective three-dimensional association is acceptable. The term may be generally applied to any nucleic acid or polypeptide that regulates the expression, processing, localization, transport, etc., of a second nucleic acid or polypeptide, generally one to which it is chemically or physically bound (e.g., covalently linked, hydrogen bonded, associated via ionic bonds).

Polymorphism: The term polymorphism refers to the occurrence of two or more alternative sequences or alleles in a population. A polymorphic site is a location at which differences in genomic DNA sequence exist among members of a population. A polymorphic variant is any of the alternate sequences or alleles that may exist at a polymorphic site among members of a population. For purposes of the present invention, the term population may refer to the population of the world, or to any subset or group of individuals. Thus the term polymorphic variant as used herein generally refers to naturally occurring variants as opposed, for example, to variants created by recombinant DNA technology. However, the term includes variants created by recombinant DNA technology when such variants replicate or duplicate naturally occurring variants. Replication or duplication of naturally occurring variants is intended to include recapitulation of a naturally occurring human variant either in a different human genetic background or in an animal model such as a mouse (e.g., the creation of a mutation at a corresponding site within mouse genomic DNA).

Typically, for the various methods described herein (e.g., diagnostic methods, methods for identifying causative mutations, etc.) described herein, it will be of interest to determine which polymorphic variant is present in a subject, among multiple polymorphic variants that exist within a population.

Peptide, polypeptide, or protein: According to the present invention, a “peptide”, “polypeptide”, or “protein” comprises a string of at least three amino acids linked together by peptide bonds. The terms may be used interchangeably although a peptide generally represents a string of between approximately 8 and 30 amino acids. Peptide may refer to an individual peptide or a collection of peptides. Peptides preferably contain only natural amino acids, although non-natural amino acids (i.e., compounds that do not occur in nature but that can be incorporated into a polypeptide chain; see, for example, the web site having URL www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide, but such modifications may confer desirable properties, e.g., enhanced biological activity, on the peptide.

A compound or agent is said to increase expression of a polypeptide if application of the compound or agent to a cell or subject results in an increase in the amount of the polypeptide. A compound or agent is said to decrease expression of a polypeptide if application of the compound or agent to a cell or subject results in a decrease in the amount of the polypeptide.

Polynucleotide or nucleic acid: Polynucleotide or nucleic acid refers to a polymer of nucleotides. The term can refer to DNA or RNA. Nucleic acids can be single stranded, double stranded, or, in some cases, triple stranded. Unless otherwise stated, nucleic acids are listed in a 5′ to 3′ direction herein. Typically, a polynucleotide comprises at least three nucleotides. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

A compound or agent is said to increase expression of a polynucleotide if application of the compound or agent to a cell or subject results in an increase in the amount of the polynucleotide or of a translation product of the polynucleotide or both. A compound or agent is said to decrease expression of a polynucleotide if application of the compound or agent to a cell or subject results in a decrease in the amount of the polynucleotide or of a translation product of the polynucleotide or both.

Primer: The term primer refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. The appropriate length of a primer depends on the intended use of the primer, but typically ranges from approximately 10 to approximately 30 nucleotides. Short primer molecules generally require lower temperatures to form sufficiently stable hybrid complexes with the template. A primer need not be perfectly complementary to the template but should be sufficiently complementary to hybridize with it. The term primer site refers to the sequence of the target DNA to which a primer hybridizes. The term “primer pair” refers to a set of primers including a 5′ (upstream) primer that hybridizes with the 5′ end of a DNA sequence to be amplified and a 3′ (downstream) primer that hybridizes with the complement of the 3′ end of the sequence to be amplified. These primers are also referred to as forward and reverse primers respectively.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

Regulatory sequence or regulatory element: The term regulatory sequence or regulatory element is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct cell type or tissue-specific and/or inducible expression. For instance, non-limiting examples of tissue-specific promoters appropriate for use in mammalian cells include lymphoid-specific promoters (see, for example, Calame et al., Adv. Immunol. 43: 235, 1988) such as promoters of T cell receptors (see, e.g., Winoto et al., EMBO J. 8: 729, 1989) and immunoglobulins (see, for example, Banerji et al., Cell 33: 729, 1983; Queen et al., Cell 33: 741, 1983), and neuron-specific promoters (e.g., the neurofilament promoter; Byrne et al., Proc. Natl. Acad. Sci. USA 86: 5473, 1989). Developmentally-regulated promoters are also encompassed, including, for example, the murine hox promoters (Kessel et al., Science 249: 374, 1990) and the α-fetoprotein promoter (Campes et al., Genes Dev. 3: 537, 1989).

Sample: As used herein, a sample obtained from a subject may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling.

The term sample also includes any material derived by isolating, purifying, and/or processing such a sample. Derived samples may include nucleic acids or proteins extracted from the sample or obtained by subjecting the sample to techniques such as amplification or reverse transcription of mRNA, etc.

A short, interfering RNA (siRNA): An siRNA comprises an RNA duplex containing two individual RNA complementary strands. The duplex portion typically ranges from about 15 to 29 base pairs in length, typically 17-23 base pairs, eg., 19 base pairs and optionally further comprises one or two single-stranded overhangs. Molecules referred to as short hairpin RNAs (shRNAs) consist of a single self-complementary RNA strand containing a similar duplex portion and further comprises a loop connecting the portions that self-hybridize. When siRNAs or shRNAs include one or more free (unhybridized) strand ends, it is generally preferred that free 5′ ends have phosphate groups, and free 3′ ends have hydroxyl groups. In certain embodiments of the invention, one strand (the antisense strand with respect to the target transcript) of the duplex portion of an siRNA (or, the self-hybridizing portion of an shRNA) is precisely complementary with a region of the target transcript, meaning that the strand hybridizes to the target transcript without a single mismatch. The overhang, if present, may also be complementary. However, in other embodiments of the invention perfect complementarity is not necessary.

An siRNA or shRNA is considered to be targeted for the purposes described herein if 1) the stability (e.g., half-life) of the target gene transcript is reduced in the presence of the siRNA or shRNA as compared with its absence; and/or 2) at least a portion (the antisense strand or portion) of the siRNA or shRNA shows at least about 80%, at least about 90%, more preferably at least about 91%, 92%, 93%, 94%, 95%, 96%, 97%, 98%, 99%, or 100% precise sequence complementarity with the target transcript for a stretch of at least 13, at least 15, at least 17, more preferably at least 18, 19, 20, 21, 22, 23, or up to 29 nucleotides; and/or 3) the siRNA or shRNA hybridizes to the target transcript under stringent conditions.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Specific binding: As used herein, the term specific binding refers to an interaction between a first molecule and a second (binding) molecule such as an antibody, agonist, or antagonist, which may be a small molecule. The interaction is typically dependent upon the presence of a particular structural feature of the first molecule such as an antigenic determinant or epitope recognized by the binding molecule (e.g., in the case of an antibody). For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. Thus the degree of specificity of an antibody will depend on the context in which it is being used. In general, an antibody exhibits specificity for a particular partner if it favors binding of that partner above binding of other potential partners. One of ordinary skill in the art will be able to select antibodies having a sufficient degree of specificity to perform appropriately in any given application (e.g., for detection of a target molecule, for therapeutic purposes, etc). In the case of binding molecules that are small molecules, interaction is also typically dependent upon the presence of a particular structural feature of the molecule to which the binding molecule binds, e.g., a cleft or three-dimensional pocket into which the small molecule fits, etc.

It is also to be understood that specificity may be evaluated in the context of additional factors such as the affinity of the binding molecule for the first molecule versus the affinity of the binding molecule for other targets, e.g., competitors. If a binding molecule exhibits a high affinity for a particular molecule that it is desired to detect and low affinity for most or all other molecules, the binding molecule will likely be an acceptable reagent, e.g., for diagnostic and/or therapeutic purposes. Once the specificity of a binding molecule is established in one or more contexts, it may be employed in other, preferably similar, contexts without necessarily re-evaluating its specificity.

Treating: As used herein, treating can include reversing, alleviating, inhibiting the progress of, preventing, and/or reducing the likelihood of the disease, disorder, or condition to which such term applies, or one or more symptoms or manifestations of such disease, disorder or condition.

Vector: The term vector is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid.

### DETAILED DESCRIPTION OF CERTAIN EMBODIMENTS

I. Overview

Calcineurin is a calcium-dependent serine/threonine protein phosphatase that plays an important role in Ca²⁺-mediated signal transduction. Since its original identification in extracts of mammalian brain, calcineurin has been implicated in a variety of biological responses, and a number of roles for calcineurin in the nervous system have been identified. Calcineurin is highly expressed in the central nervous system (Klee, C. B., Ren, H. & Wang, X. (1998) J Biol Chem 273, 13367-70; Shibasaki, F., Hallin, U. & Uchino, H. (2002) J Biochem (Tokyo) 131, 1-15; Rusnak, F. and Mertz, P., Physiological Reviews (2000) 80 (4): 1483-1522. The genomic locations of genes encoding calcineurin and many of the molecules involved in calcineurin signaling have been identified.

As described in U.S. Ser. No. 10/400,348 and in references 1 and 19, the inventors observed that forebrain-specific calcineurin B knockout mice display a spectrum of phenotypes suggestive of schizophrenia and that locations of calcineurin subunit genes and numerous other genes encoding polypeptides that play a role in calcineurin signaling are coincident with schizophrenia susceptibility loci. The inventors tested the association between certain calcineurin subunits and calcineurin interacting genes with schizophrenia and discovered direct genetic evidence that the PPP3CC gene (encoding the calcineurin A gamma subunit) is associated with schizophenia.

As discussed in more detail below, while the cause of schizophrenia has not been identified, genetic factors are known to be important. A large number of genomic regions have been identified as susceptibility loci through genetic studies, and it is believed that mutations or variations within those regions contribute to schizophrenia pathogenesis, though no individual gene, mutation, or variation has been definitively shown to play a role. The identification of specific mutations or variations that are linked to schizophrenia provides a basis for improved diagnostic tests. In addition, the identification of the particular genes in which mutation or variation contributes to schizophrenia susceptibility and/or pathogenesis not only provides a basis for improved diagnostic tests but also provides a basis for improved treatments for schizophrenia. The inventors discovered that the chromosomal locations of EGR gene family members are coincident with locations of schizophrenia loci identified through genetic linkage studies. Related to this discovery, the invention provides methods and reagents for identifying the genetic mutations or alterations that result in susceptibility to and/or development of schizophrenia and related conditions and disorders, methods and reagents for diagnosing schizophrenia or susceptibility to schizophrenia, methods and reagents for identifying compounds to prevent or treat schizophrenia, and a variety of other methods and reagents.

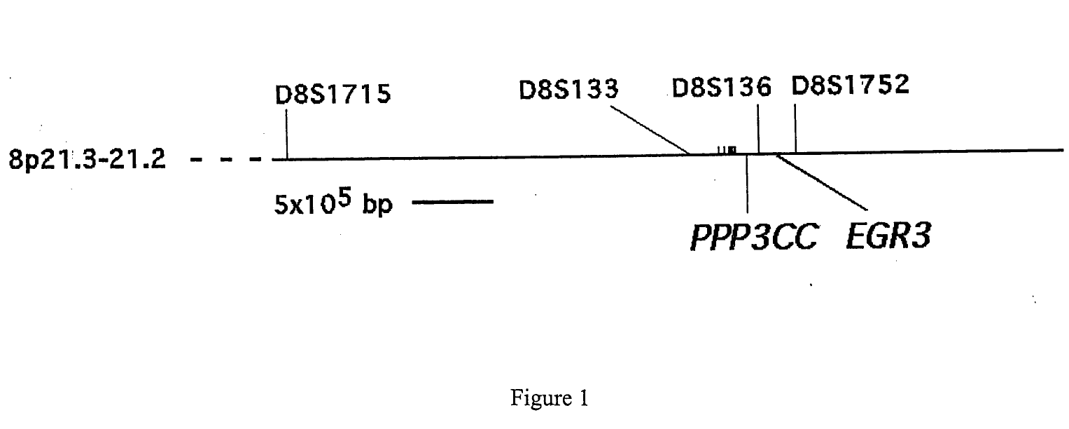

The inventors recognized that while the observed association between the PPP3CC gene and schizophrenia susceptibility was likely to reflect alterations in the PPP3CC locus itself, since the analyzed SNPs are within the PPP3CC locus, it is possible that variation affecting neighboring genes could also contribute to, or be responsible for, the observed association signal. The inventors therefore examined the human genome draft sequence to determine which genes are located in the vicinity of PPP3CC to identify candidate genes that could contribute to the observed association signal. Among the neighboring genes is EGR3, located proximal to and within 150 kilobases of PPP3CC (FIG. 1), for which the inventors have observed an association with schizophrenia, and within a confirmed schizophrenia susceptibility locus (2-8). As discussed further below, the inventors recognized a number of aspects of EGR3 function that support its potential relevance to schizophrenia pathogenesis.

EGR2 is located at chromosomal position 10q 21.3. The inventors performed a genome wide linkage scan for schizophrenia in a specific founder population sample and detected linkage of this chromosomal region with schizophrenia (Example 4). This linkage was replicated in an independent sample of families collected in the United States. EGR2 is located adjacent to the observed peak marker for linkage in both studies. Analysis with microsatellite markers and single nucleotide polymorphisms in a third independent sample consisting of 210 triads provided nominally significant evidence for association with a microsatellite and a SNP in the vicinity of the EGR2 gene.

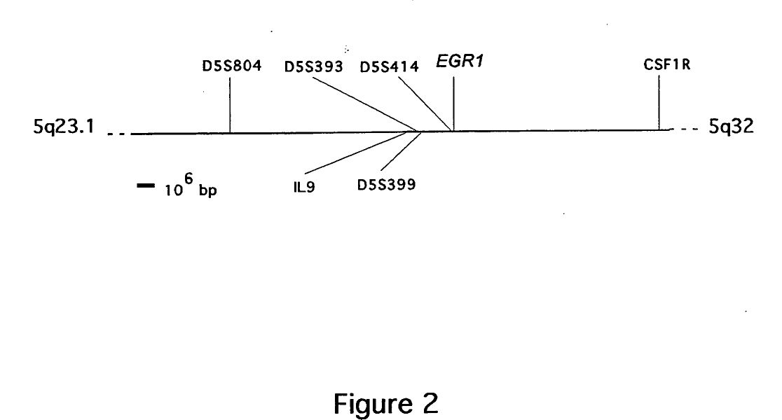

As described in more detail in Example 1, the inventors compared the positions of the other identified EGR genes with known schizophrenia susceptibility loci to determine whether these genes were also potentially linked to schizophrenia susceptibility loci and found this to be the case. Specifically, EGR1 is located at 5q31.2 (FIG. 2), within another confirmed schizophrenia susceptibility locus (8-13). EGR4 is located at 2p13.2 within a putative schizophrenia susceptibility locus at 2p13-14 (6,14-15). Thus all four EGR gene family members are located within putative schizophrenia susceptibility loci identified by linkage studies.

The coincidence in the chromosomal positions of all four EGR gene family members with locations of putative schizophrenia susceptibility loci identified by linkage or association studies suggests that altered EGR function could be a contributing factor in schizophrenia pathogenesis. The observation that EGR3 expression is induced by multiple pathways implicated in schizophrenia lends additional support. The extent and nature of involvement of EGR function in schizophrenia pathogenesis can be further confirmed by additional direct genetic studies of association of EGR genes with schizophrenia. The discoveries described herein suggest that EGR transcription factors, molecules (e.g., proteins) whose expression is induced by EGR transcription factor activity, molecules (e.g., proteins) whose activity modulates EGR function, and other genes in the EGR pathway(s) are targets for diagnosis of and therapy of schizophrenia, schizophrenia susceptibility, and schizophrenia-related conditions.

The next section discusses the EGR genes, their expression products and activities, related molecules, and molecules that either regulate or are regulated by EGR genes. Schizophrenia and its genetic basis are then described. Subsequent sections describe particular aspects of the invention in further detail.

II. EGR Molecules, EGR Interacting Molecules, and EGR Activities

A. EGR Transcription Factors

Transcriptional control of gene expression is of fundamental importance in the regulation of numerous cellular processes throughout the life of an organism. In general, transcription is regulated by the interaction of proteins referred to as transcription factors with DNA and with other nuclear proteins. Transcription factors typically bind to specific sequences within DNA (referred to as a “DNA binding site” or “DNA binding sequence”) and regulate transcription of operatively linked DNA sequences, which are frequently located downstream of (i.e., 3′ from) the DNA binding site. The DNA binding sites are typically located within promoters and/or enhancers.

While binding of transcription factors frequently activates transcription of operatively linked genes, repression may also occur. DNA binding and transcriptional activation or repression are usually mediated by distinct regions within a transcription factor. Thus many transcription factors include a DNA binding domain and a separate transcriptional activation (or repression) domain. These domains are frequently modular. For example, insertion of a DNA binding domain from a particular transcription factor into a protein that does not normally bind to DNA will frequently confer sequence-specific DNA binding activity on that protein. Similarly, insertion of a transcriptional activation (or repression) domain from a particular transcription factor into a protein that does not normally activate or repress transcription can convert the protein into a transcriptional activator or repressor, provided that the protein contains an appropriate DNA binding domain. The transcriptional activation or repression domain typically interacts with other nuclear proteins such as components of the basic transcriptional machinery (e.g., the core RNA polymerase II complex) and/or with corepressors or coactivators which may, but need not be, DNA binding proteins in their own right. The interaction is often, but need not be, via direct physical interaction. Coactivator proteins can increase the extent of transcriptional activation mediated by a transcription factor while corepressors can prevent transcriptional activation by a transcription factor and/or convert a transcription factor from one that activates transcription to one that represses transcription.

Transcription factors, coactivators, and corepressors may contain sites for binding of various ligands, which can modulate their activity. Extracellular signals are often transduced via a variety of signaling pathways that ultimately converge upon and influence transcription factors activity. For example, phosphorylation and dephosphorylation of various transcription factors, occurring as a result of extracellular stimuli, can regulate many aspects of transcription factor localization, functional activity, etc.

Members of the early growth response (EGR) family of transcription factors were discovered in searches to identify genes whose expression was induced by growth factors, which frequently trigger profound changes in cellular behavior and differentiation state. Increases in mRNA expression levels of these genes occurs rapidly following treatment by a variety of extracellular stimuli including mitogenic stimuli such as serum, platelet derived growth factors (PDGF), nerve growth factor (NGF), epidermal growth factor (EGF), fibroblast growth factor (FGF), and phorbol esters (PMA) leading to their designation as early response genes. It was later discovered that this response could also occur upon neurotransmitter receptor stimulation or depolarization, indicating that transcriptional regulation by EGR genes is likely to have various roles in addition to playing a part in differentiation and development in the nervous system.

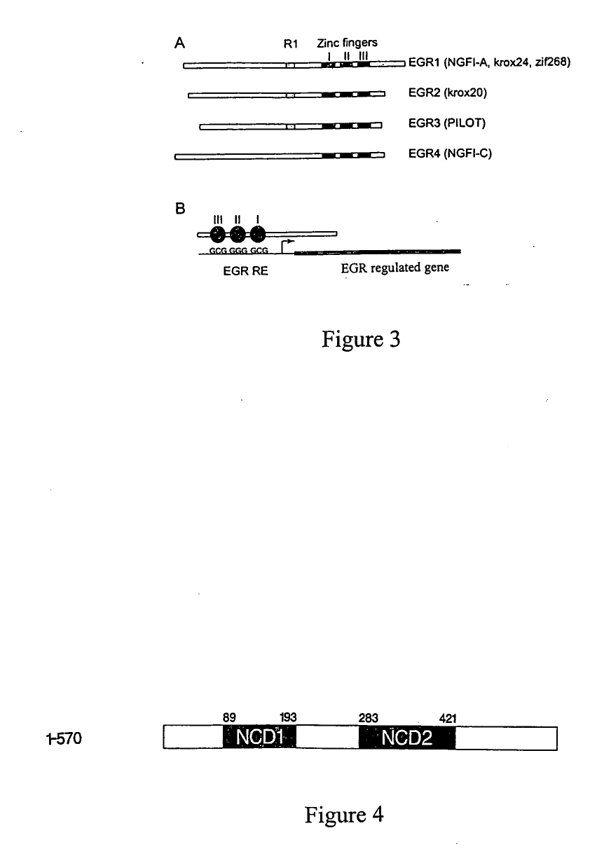

The four known EGR proteins contain three Cys₂His₂ zinc finger domains, which constitute DNA binding domains (FIG. 3A). These domains bind to a GC-rich consensus binding site, e.g., GCG[G/T]GGGCG, (FIG. 3B)(16) (the nucleotide at position 4 is usually a G). One or more copies of this DNA binding sequence, or variants thereof, is found in a number of genes, typically upstream of the coding region, and has been shown to allow regulation of the transcription these genes by EGR family members, which display similar binding specificities (16, and references therein). An operatively linked gene or DNA sequence whose transcription is regulated (e.g., activated or repressed) by an EGR transcription factor may be referred to as an “EGR-regulated gene”. Confirmed or putative EGR regulated genes include transforming growth factor β, platelet-derived growth factor (PDGF) A and B chains, tissue factor, Fas ligand, various Hox genes, stromelysin, ICAM-1, IL-2, IL2Rβ, CD44, tumor necrosis factor α, and luteneizing hormone β (LHβ). EGR DNA binding sites found upstream of these genes have been identified (see 69 and references therein). Noncanonical EGR1 binding sites that mediate transcriptional activation by EGR1 have been identified in the regulatory regions of various other genes, e.g., IL-2Rβ (GCGTAGGAGGCA —SEQ ID NO:1), PDGF A chain (GAGGAGGAGGAGGA—SEQ ID NO:2), rat cardiac myosin heavy chain (GTGGGGGTG), and fasL (AAGTGAGTGGGTGTTT—SEQ ID NO:3) (and references therein), thymidine kinase gene (CCGTGGGTG). Genes containing an EGR DNA binding site are putative EGR-regulated genes. It has also been discovered that the Wilms tumor suppressor gene (WT1) (chromosomal location 11p13), is a zinc finger polypeptide containing four zinc finger domains, of which domains II-IV are homologous to the EGR zinc fingers. In addition, the consensus DNA target sequence of WT1, GCGTGGGAGT, is related to the consensus EGR DNA binding site.

Deficiencies in EGR family members have been implicated as playing a causative role in various abnormalities (30 and references therein). In particular, EGR1 deficiency, leading to a reduction in pituitary LHβ, is implicated in female infertility. Other endocrine defects were also found in mice lacking EGR1 expression. Mice deficient in EGR2 display severe abnormalities in hindbrain development and peripheral nerve myelination. Mutations in the gene encoding EGR2 have been found in patients with congenital abnormalities of peripheral nerve myelination (e.g., Charcot-Marie-Tooth type 1, Dejerine-Sottas syndrome, and congenital hypomyelinating neuropathy). EGR3 deficient mice display severe motor abnormalities, most likely as a consequence of the fact that they lack muscle spindles. These mice display sensory ataxia, scoliosis, tremor, and ptosis. EGR4 deficiency has been shown to cause male infertility and an autonomous germ cell defect. Compounds that modulate EGR level and/or functional activity may be useful in the treatment of diseases and conditions associated with the afore-mentioned symptoms and signs. In addition to playing a role in these diverse aspects of development and disease, EGR transcription factors are believed to participate in changes in gene expression that underlie synaptic plasticity. Compounds that modulate EGR level and/or functional acitivy may therefore be useful in improving learning and/or memory, or in treating disorders of learning and/or memory.

B. EGR Regulation and Interacting Proteins

Mutational analysis of EGR1 identified an inhibitory domain, referred to as R1, deletion of which results in enhanced EGR1 transcriptional activity (FIG. 3A). The domain has been defined as extending from amino acid 267 to 306 of EGR1. Homologous R1 domains are present in EGR2 and EGR3. A two-hybrid screen in yeast, performed using the EGR R1 domain as bait, resulted in identification of a protein named NAB1, which interacts with EGR1 in vitro and represses EGR1-mediated transcription in cells (68, and references therein). A related protein, NAB2, which shares significant homology with NAB1 and also interacts with EGR proteins via the R1 domain, was subsequently discovered. A mutation at position 293 (e.g., an 1293A point mutation) in the R1 domain prevents EGR1 interaction with NAB proteins. The R1 domain is portable, in that the presence of this domain in proteins that are otherwise not susceptible to NAB-mediated repression can be made susceptible by inserting an R1 domain into them.

A schematic diagram of NAB1 is shown in FIG. 4. NAB1 and NAB2 bind to the R1 domain via a region referred to as NCD1, which is conserved between NAB1 and NAB2. A second conserved region in NAB 1 and NAB2, referred to as NCD2, represses EGR-mediated transcription (68). NAB proteins do not appear to interact with EGR4, which lacks the R1 domain. NAB-mediated repression is believed to involve recruitment of NAB-EGR complexes to regulatory regions (e.g., promoters) containing EGR DNA binding sites, though NAB proteins can also repress certain promoters without requiring EGR proteins. While NAB proteins generally repress EGR-mediated transcription, at certain promoters such as the LHβ and Fas ligand promoters, NAB proteins instead stimulate EGR-directed transcription (69). Analysis of a number of synthetic and mutant promoters indicated that the strength (binding affinity) and multiplicity of EGR DNA binding sites determines whether NAB proteins behave as transcriptional corepressors (repressing EGR-mediated transcription) or transcriptional coactivators (enhancing EGR-mediated transcription). Thus manipulating the number and sequence (which influences binding activity) of EGR binding sites can result in either corepression or coactivation of EGR-mediated transcription of an operatively linked gene.

C. Functional Relevance of EGR3 to Schizophrenia

While not wishing to be bound by any theory, the inventors note a number of functional aspects of EGR3 that are consistent with its potential involvement in schizophrenia pathogenesis. First, EGR3 expression is induced by calcineurin signaling (16). Second, EGR3 expression is induced by neuronal activity (17), NMDA receptor activation (17) and drugs that alter dopaminergic transmission (17). In addition, EGR3 expression has been shown to be induced by Neuregulinl/ErbB signaling (18). Alterations in calcineurin signaling (1,19, and pending patent application, U.S. Ser. No. 10/400,348), NMDA receptor signaling (20), dopaminergic transmission (21-25) and neuregulinl signaling (26) have all been implicated in schizophrenia pathogenesis. Thus EGR3 interacts with several molecular pathways reported to be involved with schizophrenia etiology.

D. EGR Molecules and EGR Interacting Molecules

As is evident from the description above, EGR transcription factors regulate and are regulated by expression products of a wide variety of genes. While not wishing to be bound by any theory, the fact that EGR proteins are transcription factors suggests that alterations in their expression level and/or functional activity (e.g., alterations that may result from mutations in the coding and/or regulatory sequences of EGR family members) will be reflected in alterations in the expression of EGR-regulated genes. Such alterations in the expression of EGR-regulated genes would likely be the means by which mutations in EGR genes contribute to schizophrenia susceptibility. Alterations in the expression level and/or functional activity of genes whose products regulate EGR proteins would be expected to alter EGR functional activity and could thus contribute to schizophrenia susceptibility by altering the expression of EGR-regulated genes. Therefore, mutations in either genes whose expression products either directly (e.g., by physically interacting with) or indirectly regulate EGR expression, and genes whose expression regulated by EGR may also contribute to schizophrenia susceptibility. Such genes and their expression products, referred to as EGR interacting molecules, are also targets for diagnosis and/or treatment of schizophrenia. As used herein, the phrase “EGR molecule”, refers to molecules (RNA or protein) encoded by the EGR1, EGR2, EGR3, or EGR4 genes (e.g., EGR1, EGR2, EGR3, or EGR4 protein). It is noted that the teachings of the invention may encompass molecules encoded by the WT1 gene, which has a DNA binding specificity related in sequence to the DNA binding sequence of EGR genes and may therefore play a role in regulating EGR-regulated genes.

As used herein, the phrase “EGR interacting molecule” refers to molecules (RNA or protein) that alter or modulate (e.g., enhance or inhibit) functional activity of one or more EGR molecules; molecules that regulate EGR expression (which includes regulation of expression of any EGR molecule or other EGR interacting molecule), intracellular location, and/or functional activity; RNAs or proteins whose expression is regulated by EGR (i.e., expression products of EGR-regulated genes); molecules that modify and/or post-translationally process an EGR molecule and/or an EGR interacting molecule; and molecules that enhance or antagonize the effects of an EGR molecule. “Regulation of expression” of an EGR molecule or an EGR interacting molecule may include regulation of transcription and/or post-transcriptional processing (e.g., splicing, polyadenylation) and/or localization of transcripts that encode such molecules, regulation of translation of transcripts that encode such molecules, and regulation of the degradation of transcripts that encode such molecules or degradation of the molecules themselves.

It will thus be appreciated that the term “EGR interacting molecule” includes but is not limited to, molecules that physically interact with an EGR molecule. Such molecules may be preferred for certain purposes. As described herein, certain of the genes that encode EGR molecules, and/or EGR interacting molecules are coincident with previously mapped or identified schizophrenia susceptibility loci. Without intending to limit the invention to such molecules, this subset includes the following: EGR1, EGR2, EGR3, and EGR4. These molecules and others are listed in Table 1 together with their GenBank accession numbers, names (and alternate names), and chromosomal locations. The fact that to date no schizophrenia locus that is coincident or nearby the chromosomal location of certain EGR interacting molecules may simply reflect the fact that genetic studies have thus far only identified a subset of susceptibility loci.

TABLE 1
Gene symbols, accession numbers, names, and
chromosomal locations of EGR genes and genes
encoding EGR interacting molecules.

| Gene | Accession | | Chromosomal |
|---|---|---|---|
| Symbol | Number | Name(s) | location |
| EGR1 | NM_001964 | Early growth response 1, | 5q31.2 |
| | | NGFI-A, Krox-24, zif268, TIS-8 | |
| EGR2 | NM_000399 | Early growth response 2, | 10q21.3 |
| | | NGFI-B, Krox20 | |
| EGR3 | NM_004430 | Early growth response 3, | 8p21.3 |
| | | PILOT | |
| EGR4 | NM_001965 | Early growth response 4, | 2p13.2 |
| | | NGFI-C | |
| NAB1 | NM_005966 | NGFI-A binding protein 1 | 2q32.3-33 |
| NAB2 | NM_005967 | NGFI-A binding protein 2 | 12q13.3-14.1 |

III. Genetic Analysis of Schizophrenia and Related Conditions

The cause of schizophrenia is unknown, but it has been shown to include a significant genetic component. Unlike disorders such as cystic fibrosis and sickle cell anemia that exhibit a Mendelian inheritance pattern and are caused by mutations in a single gene, schizophrenia is believed to be a multigenic disorder in which mutations or variations in many different genes may contribute, to different degrees and in different combinations, to development of disease. It appears likely that contributions from multiple genes are involved in any given patient, and that mutations or alterations in these genes display varying degrees of penetrance so that even if an individual harbors a mutation or alteration that may contribute to schizophrenia pathogenesis, the individual may not develop clinical disease. These features have made it difficult to conclusively determine the genetic basis of schizophrenia.

A large number of genetic studies have implicated certain regions of genomic DNA (chromosomal locations) as possibly harboring mutations or variations that contribute to development of schizophrenia Karayiorgou, M. & Gogos, J. A. (1997) Neuron 19, 967-79; Thaker, G. K. & Carpenter, W. T., Jr. (2001) Nat Med 7, 667-71). These regions are typically on the order of many kilobases or megabases in length and are referred to as “susceptibility loci” to reflect the fact that mutations or alterations somewhere within these regions are believed to confer an increased likelihood that an individual having such mutations or alterations will develop the condition. It is therefore likely that such regions harbor genes which, alone or in combinations, are causally implicated in schizophrenia in at least a subset of patients. Genetic studies include linkage studies, in which families having an increased incidence of schizophrenia relative to the incidence in the general population (referred to herein as “schizophrenia families” are studied) and association studies, in which populations typically containing both related and unrelated subjects diagnosed with schizophrenia, e.g., groups of schizophrenia families, are studied. Association studies can compare the frequencies of certain haplotypes in control and affected populations. Alternately, they can assess disequilibrium in the transmission of certain haplotypes to affected probands. In accordance with the art-accepted definition, a “haplotype” can be a specific polymorphic variant for a given polymorphism on a single chromosome, or the combination of polymorphic variants (alleles) for a group of polymorphisms represented on a single chromosome for a particular individual.

Linkage and association studies typically make use of genetic polymorphisms, i.e., differences between genomic DNA sequence that exist among members of a population at certain locations in the genome. (See, e.g., Cardon, L. and Bell, J., (2001), Nature Reviews Genetics, Vol. 2, pp. 91-99; Kruglyak, L. and Lander, E. (1995), Am. J. Hum. Genet., 56: 1212-1223; Jorde, L. B. (2000), Genome Research, 10: 1435-1444; Pritchard, J. and Przeworski, M. (2001), Am. J. Hum. Genet., 69: 1-14 and references in the foregoing articles for discussion of considerations in design of genetic studies, particularly for complex traits and diseases in which multiple genes play a role, such as schizophrenia). For example, a population may contain multiple subpopulations of individuals each of which has a different DNA sequence at a particular chromosomal location. Such polymorphisms may be single nucleotide differences (single nucleotide polymorphisms, referred to herein as SNPs). (See, e.g., Nowotny, P., et al. (2001), Curr. Op. Neurobiol., 11: 637-641; Wall, J. (2001), 11: 647-651 and references in these articles.) When SNPs occur within coding regions they may, but frequently do not, result in alterations in the amino acid sequence of the encoded protein. In general, while not wishing to be bound by any theory, SNPs are thought to arise as a result of mutations in what was originally a more homogeneous ancestral sequence. Other polymorphisms include multiple nucleotide polymorphisms, deletions (including microdeletions), insertions, inversions, translocations, etc.

It will be appreciated that while certain polymorphic variants may be responsible for disease or phenotypic variation by, for example, causing a functional alteration in an encoded protein, many polymorphisms appear to be silent in that no known detectable difference in phenotype exists between individuals having different alleles. However, polymorphisms (whether silent or not) may be physically and/or genetically linked to genes or DNA sequences in which mutations or variations confer susceptibility to and/or play a causative role in disease (i.e., they are located within a contiguous piece of DNA). In the absence of genetic recombination, polymorphisms that are physically linked to such mutations or variations will generally be inherited together with the mutation or alteration.

With increasing genetic recombination between any given polymorphism and a causative mutation or variation, the extent of co-inheritance will be reduced. Since the likelihood of genetic recombination between loci generally increases with increasing distance between the loci (though not necessarily in a linear fashion), co-inheritance of a particular polymorphism and a particular phenotype suggests that the polymorphism is located in proximity to a causative mutation or variation. Thus studying the co-inheritance of polymorphic variants, e.g., SNPs, allows identification of genomic regions likely to harbor a mutation or variation that, alone or in combination with other mutations or variations, causes or increases susceptibility to disease. Polymorphisms are thus useful for genetic mapping and identification of candidate genes, in which mutations or variations may play a causative role in disease. In addition, detection of particular polymorphic variants (alleles) is useful for diagnosis of disease or susceptibility to disease as described herein.

Linkage and association studies have identified a large number of schizophrenia susceptibility loci as described in a number of the references and in United States Published Patent Application 20020165144. These references are merely representative, and one of ordinary skill in the art will be able to perform literature searches to learn of additional such loci. In addition, a number of candidate genes located near or within schizophrenia susceptibility regions identified from genetic studies have been suggested to play a role in the etiology of schizophrenia. (See, e.g., Straub, R. E., et al., Am. J. Hum. Genet. (2002) 71: 337-348; Stefannson, H., et al., Am. J. Hum. Genet. (2002) 71: 877-892). However, definitive proof of the involvement of any of these candidate genes in schizophrenia is lacking.

Schizophrenia is one of a group of psychiatric conditions and disorders that exhibit a spectrum of similar phenotypes. Many of these conditions and disorders are found at increased frequency in family members of schizophrenic subjects, relative to their incidence in the general population. These factors make it likely that the same genetic mutations or alterations that contribute to schizophrenia susceptibility and/or pathogenesis are also involved in susceptibility to and/or pathogenesis of these conditions and disorders. Thus the methods and reagents of the invention are also applicable to these related conditions and disorders.

Conditions related to schizophrenia may include, but are not limited to: schizoaffective disorder, schizotypal personality disorder, schizotypy, atypical psychotic disorders, avoidant personality disorders, bipolar disorder, attention deficit hyperactivity disorder (ADHD), and obsessive compulsive disorder (OCD). Features and diagnostic criteria for these conditions are defined in DSM-III, DSM III-R, DSM-IV, or DSM IV-R. For purposes of description, rather than referring to “schizophrenia and/or related conditions or disorders”, the invention will be described in terms of schizophrenia itself. However, it is to be understood that the methods, e.g., diagnostic and therapeutic methods, and reagents may also be used in a similar manner with respect to these conditions and disorders as described for schizophrenia itself. Similarly, compounds identified as potential prophylactic or therapeutic agents for schizophrenia may also be utilized for treatment and/or prevention of these related disorders. The following sections provide further description of the various aspects of the invention.

IV. Methods and Reagents for Diagnosis of Schizophrenia or Schizophrenia Susceptibility

A. Diagnostic Methods. The invention provides a variety of methods for the diagnosis of schizophrenia or schizophrenia susceptibility. In particular, the invention provides a method for the diagnosis of schizophrenia or schizophrenia susceptibility comprising: (i) providing a sample obtained from a subject to be tested for schizophrenia or schizophrenia susceptibility; and (ii) detecting a polymorphic variant of a polymorphism in a coding or noncoding portion of gene encoding an EGR molecule or an EGR interacting molecule, or detecting a polymorphic variant of a polymorphism in a genomic region linked to a coding or noncoding portion of a gene encoding an EGR molecule or an EGR interacting molecule, in the sample. It is to be understood that “susceptibility to schizophrenia” does not necessarily mean that the subject will develop schizophrenia but rather that the subject is, in a statistical sense, more likely to develop schizophrenia than an average member of the population. As used herein, “susceptibility to schizophrenia” may exist if the subject has one or more genetic determinants (e.g., polymorphic variants or alleles) that may, either alone or in combination with one or more other genetic determinants, contribute to an increased risk of developing schizophrenia in some or all subjects. Ascertaining whether the subject has any such genetic determinants (i.e., genetic determinants that may increase the risk of developing schizophrenia in the appropriate genetic background) is included in the concept of diagnosing susceptibility to schizophrenia as used herein. Such determination is useful, for example, for purposes of genetic counseling. Thus providing diagnostic information regarding schizophrenia susceptibility includes providing information useful in genetic counseling, and the provision of such information is encompassed by the invention.

The sample itself will typically consist of cells (e.g., blood cells), tissue, etc., removed from the subject. The subject can be an adult, child, fetus, or embryo. According to certain embodiments of the invention the sample is obtained prenatally, either from the fetus or embryo or from the mother (e.g., from fetal or embryonic cells in that enter the maternal circulation). The sample may be further processed before the detecting step. For example, DNA in the cell or tissue sample may be separated from other components of the sample, may be amplified, etc. All samples obtained from a subject, including those subjected to any sort of further processing, are considered to be obtained from the subject.

In general, if the polymorphism is located in a gene, it may be located in a noncoding or coding region of the gene. If located in a coding region the polymorphism may, but frequently will not, result in an amino acid alteration. Such alteration may or may not have an effect on the function or activity of the encoded polypeptide. If the polymorphism is linked to, but not located within, a gene, it is preferred that the polymorphism is closely linked to the gene. For example, it is preferred that the recombination frequency between the polymorphism and the gene is less than approximately 20%, preferably less than approximately 10%, less than approximately 5%, less than approximately 1%, or still less.

According to certain preferred embodiments of any of the inventive methods described above, the gene is coincident with a mapped or identified schizophrenia susceptibility locus. For example, according to various embodiments of the invention the gene may encode EGR1, EGR2, EGR3, or EGR4, each of which is coincident with a mapped or identified schizophrenia susceptibility locus. The inventive methods also encompass genes coincident with schizophrenia susceptibility loci that have yet to be mapped or identified. By “coincident with” is meant either that the gene or a portion thereof falls within the identified chromosomal location or is located in close proximity to that location. In general, the resolution of studies identifying genetic susceptibility loci may be on the order of tens of centimorgans. According to certain embodiments of the invention “close proximity” refers to within 20 centimorgans of either side of the susceptibility locus, more preferably within 10 centimorgans of either side of the susceptibility locus, yet more preferably within 5 centimorgans of either side of the susceptibility locus. In general, susceptibility loci are designated by the chromosomal band positions that they span (e.g., 8p21 refers to chromosome 8, arm p, band 21; 8p20-21 refers to chromosome 8, arm p, bands 20-21 inclusive) and may be defined at higher resolution (e.g., 8p21.1). In general, the terms “coincident with” and “close proximity” may be interpreted in light of the knowledge of one of ordinary skill in the art.

Genes that are expressed in the nervous system, e.g., in the brain, may be particularly attractive candidates as schizophrenia susceptibility genes. Such genes may be expressed throughout the brain or in particular regions or cell types or regions in the brain such as cell types or regions (e.g., forebrain, cortex, hippocampus, etc.) implicated in schizophrenia pathogenesis. However, it is possible that genes not currently recognized as expressed in the brain will prove important. For example, such genes may be expressed in only a small subset of brain cells, during particular developmental stages, in particular environmental conditions, etc. Schizophrenia susceptibility genes may also be expressed outside the brain in addition to, or instead of, within the brain.

The invention further provides a method for the diagnosis of schizophrenia or schizophrenia susceptibility comprising: (i) providing a sample obtained from a subject to be tested for schizophrenia or schizophrenia susceptibility; and (ii) detecting an alteration or variation in expression or activity of an EGR molecule or an EGR interacting molecule, in the sample, relative to the expression or activity of the EGR molecule or EGR interacting molecule that would be expected in a sample obtained from a normal subject. For example, according to various embodiments of the invention the gene may encode any of the molecules listed in Table 1. The gene may be an EGR-regulated gene. Such genes include, but are not limited to: transforming growth factor β, platelet-derived growth factor (PDGF) A and B chains, tissue factor, Fas ligand, Hox genes, stromelysin, thymidine kinase, ICAM-1, IL-2, IL2Rβ, CD44, tumor necrosis factor α, and luteneizing hormone β (LHβ).

According to certain embodiments of any of the inventive methods for diagnosis, the methods are applied before the disease or condition manifests clinically. This may be advantageous for early intervention. Appropriate therapy may be administered to a susceptible subject (or to the subject's mother in the case of prenatal diagnosis) prior to development of disease (e.g., prior to birth in the case of prenatal diagnosis). Since schizophrenia may be at least in part a developmental disorder, such early intervention may prove to be critical for prevention of the disease.

The following sections provide further details regarding particular embodiments of the inventive methods and reagents. It is to be understood that there are not intended to be limiting.

B. Methods and Reagents for Identification and Detection ofpolymorphisms.

In general, polymorphisms of use in the practice of the invention may be initially identified using any of a number of methods well known in the art. For example, numerous polymorphisms are known to exist and are available in public databases, which can be searched as described, for example, in Example 1. Alternately, polymorphisms may be identified by sequencing either genomic DNA or cDNA in the region in which it is desired to find a polymorphism. According to one approach, primers are designed to amplify such a region, and DNA from a subject suffering from schizophrenia is obtained and amplified. The DNA is sequenced, and the sequence (referred to as a “subject sequence”) is compared with a reference sequence, which is typically taken to represent the “normal” or “wild type” sequence. Such a sequence may be, for example, the human draft genome sequence, publicly available in various databases mentioned in Example 1, or a sequence deposited in a database such as GenBank. In general, if sequencing reveals a difference between the sequenced region and the reference sequence, a polymorphism has been identified. Note that this analysis does not necessarily presuppose that either the subject sequence or the reference sequence is the “normal”, most common, or wild type sequence. It is the fact that a difference in nucleotide sequence is identified at a particular site that determines that a polymorphism exists at that site. In most instances, particularly in the case of SNPs, only two polymorphic variants will exist at any location. However, in the case of SNPs, up to four variants may exist since there are four naturally occurring nucleotides in DNA. Other polymorphisms such as insertions may have more than four alleles.

Once a polymorphic site is identified, any of a variety of methods may be employed to detect the existence of any particular polymorphic variant in a subject. In general, a subject may have either the reference sequence or an alternate sequence at the site. The phrase “detecting a polymorphism” or “detecting a polymorphic variant” as used herein generally refers to determining which of two or more polymorphic variants exists at a polymorphic site, although “detecting a polymorphism” may also refer to the process of initially determining that a polymorphic site exists in a population. The meaning to be given to these phrases will be clear from the context as interpreted in light of the knowledge of one of ordinary skill in the art. For purposes of description, if a subject has any sequence other than a defined reference sequence (e.g. the sequence present in the human draft genome) at a polymorphic site, the subject may be said to exhibit the polymorphism. In general, for a given polymorphism, any individual will exhibit either one or two possible variants at the polymorphic site (one on each chromosome). (This may, however, not be the case if the individual exhibits one more chromosomal abnormalities such as deletions.)

Detection of a polymorphism or polymorphic variant in a subject (genotyping) may be performed by sequencing, similarly to the manner in which the existence of a polymorphism is initially established as described above. However, once the existence of a polymorphism is established a variety of more efficient methods may be employed. Many such methods are based on the design of oligonucleotide probes or primers that facilitate distinguishing between two or more polymorphic variants.

“Probes” or “primers”, as used herein, typically refers to oligonucleotides that hybridize in a base-specific manner to a complementary nucleic acid molecule. Such probes and primers include polypeptide nucleic acids, as described in Nielsen et al, Science, 254, 1497-1500 (1991). The term “primer” in particular generally refers to a single-stranded oligonucleotide that can act as a point of initiation of template-directed DNA synthesis using methods such as PCR (polymerase chain reaction), LCR (ligase chain reaction), etc. Typically, a probe or primer will comprise a region of nucleotide sequence that hybridizes to at least about 8, more often at least about 10 to 15, typically about 20-25, and frequently about 40, 50 or 75, consecutive nucleotides of a nucleic acid molecule. In certain embodiments of the invention, a probe or primer comprises 100 or fewer nucleotides, preferably from 6 to 50 nucleotides, preferably from 12 to 30 nucleotides. In certain embodiments of the invention, the probe or primer is at least 70% identical to the contiguous nucleotide sequence or to the complement of the contiguous nucleotide sequence, preferably at least 80% identical, more preferably at least 90% identical, even more preferably at least 95% identical, or having an even higher degree of identity. In certain embodiments of the invention a preferred probe or primer is capable of selectively hybridizing to a target contiguous nucleotide sequence or to the complement of the contiguous nucleotide sequence. According to certain embodiments of the invention a probe or primer further comprises a label, for example by incorporating a radioisotope, fluorescent compound, enzyme, or enzyme co-factor.

Oligonucleotides that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site within it or at one or the other end) will generally hybridize preferentially to a nucleic acid comprising that sequence as opposed to a nucleic acid comprising an alternate polymorphic variant.

In order to detect polymorphisms and/or polymorphic variants, it will frequently be desirable to amplify a portion of DNA encompassing the polymorphic site. Such regions can be amplified and isolated by PCR using oligonucleotide primers designed based on genomic and/or cDNA sequences that flank the site. See e.g., PCR Primer: A Laboratory Manual, Dieffenbach, C. W. and Dveksler, G. S. (Eds.); PCR Basics: From Background to Bench, Springer Verlag, 2000; M. J. McPherson, et al; Mattila et al., Nucleic Acids Res., 19: 4967 (1991); Eckert et al., PCR Methods and Applications, 1: 17 (1991); PCR (eds. McPherson et al., IRL Press, Oxford); and U.S. Pat. No. 4,683,202. Other amplification methods that may be employed include the ligase chain reaction (LCR) (Wu and Wallace, Genomics, 4: 560 (1989), Landegren et al., Science, 241: 1077 (1988), transcription amplification (Kwoh et al., Proc. Natl. Acad. Sci. USA, 86: 1173 (1989)), self-sustained sequence replication (Guatelli et al., Proc. Nat. Acad. Sci. USA, 87: 1874 (1990)), and nucleic acid based sequence amplification (NASBA). Guidelines for selecting primers for PCR amplification are well known in the art. See, e.g., McPherson, M., et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

According to certain methods for diagnosing schizophrenia or susceptibility to schizophrenia, hybridization methods, such as Southern analysis, Northern analysis, or in situ hybridizations, can be used (see Current Protocols in Molecular Biology, Ausubel, F. et al., eds., John Wiley & Sons). For example, a sample (e.g., a sample comprising genomic DNA, RNA, or cDNA), is obtained from a subject suspected of being susceptible to or having schizophrenia. The DNA, RNA, or cDNA sample is then examined to determine whether a polymorphic variant in a coding or noncoding portion of a gene encoding a calcineurin subunit or a calcineurin interacting molecule, or a polymorphic variant in a genomic region linked to a coding or noncoding portion of a gene encoding an EGR molecule or an EGR interacting molecule, is present. The presence of the polymorphic variant can be indicated by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant, e.g., a polymorphic variant indicative of susceptibility to schizophrenia.

In order to diagnose susceptibility to schizophrenia, a hybridization sample is formed by contacting the sample with at least one nucleic acid probe. The probe is typically a nucleic acid probe (which may be labeled, e.g., with a radioactive, fluorescent, or enzymatic label or tag) capable of hybridizing to mRNA, genomic DNA, and/or cDNA sequences encompassing a polymorphic site in a coding or noncoding portion of gene encoding an EGR molecule or an EGR interacting molecule, or encompassing a polymorphic variant in a genomic region linked to a coding or noncoding portion of a gene encoding an EGR molecule or an EGR interacting molecule. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA, cDNA, or genomic DNA.

The hybridization sample is maintained under conditions selected to allow specific hybridization of the nucleic acid probe to a region encompassing the polymorphic site. Specific hybridization can be performed under high stringency conditions or moderate stringency conditions, for example, as described above. In a particularly preferred embodiment, the hybridization conditions for specific hybridization are high stringency. In general, the probe may be perfectly complementary to the region to which it hybridizes, i.e., perfectly complementary to a region encompassing the polymorphic site when the site contains any particular polymorphic sequence. Multiple nucleic acid probes (e.g., multiple probes differing only at the polymorphic site, or multiple probes designed to detect polymorphic variants at multiple polymorphic sites) may be used concurrently in this method. Specific hybridization of any one of the nucleic acid probes is indicative of a polymorphic variant in a coding or noncoding portion of gene encoding an EGR molecule or an EGR interacting molecule, or is indicative of a polymorphic variant in a genomic region linked to a coding or noncoding portion of a gene encoding an EGR molecule or an EGR interacting molecule, and is thus diagnostic of susceptibility to schizophrenia.

Northern analysis may be performed using similar nucleic acid probes in order to detect a polymorphic variant of a polymorphism in a coding or noncoding portion of gene encoding an EGR molecule or an EGR interacting molecule, or detecting a polymorphic variant in a genomic region linked to a coding or noncoding portion of a gene encoding an EGR molecule or an EGR interacting molecule. See, e.g., Ausubel, Current Protocols in Molecular Biology, referenced above.

According to certain embodiments of the invention, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a nucleic acid comprising a polymorphic variant conferring susceptibility to or indicative of the presence of schizophrenia.

According to another method, restriction digest analysis can be used to detect the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Current Protocols in Molecular Biology, referenced above). The digestion pattern of the relevant DNA fragment indicates the presence or absence of a particular polymorphic variant of the polymorphism and is therefore indicative of the presence or absence of susceptibility to schizophrenia.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to detect the presence of a polymorphic variant, e.g., through the use of dot-blot hybridization of amplified oligonucleotides with allele-specific oligonucleotide (ASO) probes (see, for example, Saiki, R. et al., (1986), Nature (London) 324: 163-166). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism, e.g., a polymorphism associated with a susceptibility to schizophrenia. An allele-specific oligonucleotide probe that is specific for particular a polymorphism can be prepared, using standard methods (see Current Protocols in Molecular Biology).

To determine which of multiple polymorphic variants is present in a subject, a sample comprising DNA is obtained from the individual. PCR can be used to amplify a portion encompassing the polymorphic site. DNA containing the amplified portion may be dot-blotted, using standard methods (see Current Protocols in Molecular Biology), and the blot contacted with the oligonucleotide probe. The presence of specific hybridization of the probe to the DNA is then detected. Specific hybridization of an allele-specific oligonucleotide probe (specific for a polymorphic variant indicative of susceptibility to schizophrenia) to DNA from the subject is indicative of susceptibility to schizophrenia.

According to another embodiment of the invention, arrays of oligonucleotide probes that are complementary to nucleic acid portions from a subject can be used to identify polymorphisms. Oligonucleotide arrays typically comprise a plurality of different oligonucleotide probes that are coupled to a surface of a substrate in different known locations. These oligonucleotide arrays, also referred to as “Genechips™” are described, for example, in U.S. Pat. No. 5,143,854 and PCT patent publication Nos. WO 90/15070 and 92/10092. Such arrays can generally be produced using mechanical synthesis methods or light directed synthesis methods which incorporate a combination of photolithographic methods and solid phase oligonucleotide synthesis methods. See Fodor et al., Science, 251: 767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261, the entire teachings of which are incorporated by reference herein.

The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., Published PCT Application Nos. WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition, to oligonucleotide arrays, cDNA arrays may be used similarly in certain embodiments of the invention.

Other methods of nucleic acid analysis can be used to detect polymorphisms and/or polymorphic variants. Such methods include, e.g., direct manual sequencing (Church and Gilbert, (1988), Proc. Natl. Acad. Sci. USA 81: 1991-1995; Sanger, F. et al. (1977) Proc. Natl. Acad. Sci. 74: 5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al (19891) Proc. Natl. Acad. Sci. USA 86: 232-236), mobility shift analysis (Orita, M. et al. (1989) Proc. Natl. Acad. Sci. USA 86: 2766-2770), restriction enzyme analysis (Flavell et al. (1978) Cell 15: 25; Geever, et al. (1981) Proc. Natl. Acad. Sci. USA 78: 5081); heteroduplex analysis; chemical mismatch cleavage (CMC) (Cotton et al. (1985) Proc. Natl. Acad. Sci. USA 85: 4397-4401); RNase protection assays (Myers, R. M. et al. (1985) Science 230: 1242); use of polypeptides that recognize nucleotide mismatches, e.g., E. coli mutS protein; allele-specific PCR, for example.

In certain embodiments of the invention fluorescence polarization template-directed dye-terminator incorporation (FP-TDI) is used to determine which of multiple polymorphic variants of a polymorphism is present in a subject. This method is based on template-directed primer extension and detection by fluorescence polarization. According to this method, amplified genomic DNA containing a polymorphic site is incubated with oligonucleotide primers (designed to hybridize to the DNA template adjacent to the polymorphic site) in the presence of allele-specific dye-labeled dideoxyribonucleoside triphosphates and a commercially available modified Taq DNA polymerase. The primer is extended by the dye-terminator specific for the allele present on the template, increasing ˜10-fold the molecular weight of the fluorophore. At the end of the reaction, the fluorescence polarization of the two dye-terminators in the reaction mixture are analyzed directly without separation or purification. This homogeneous DNA diagnostic method has been shown to be highly sensitive and specific and is suitable for automated genotyping of large number of samples. (Chen, X., et al., Genome Research, Vol. 9, Issue 5, 492-498, 1999). Note that rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderbom, A., et al., Genome Research, Vol. 10, Issue 8, 1249-1258, 2000). Additional methods include, for example, PCR amplification in combination with denaturing high performance liquid chromatography (dHPLC) (Underhill, P. A., et al., Genome Research, Vol. 7, No. 10, pp. 996-1005, 1997).

In general, it will be of interest to determine the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means above may be used to determine the genotype of a subject with respect to one or both copies of the polymorphism present in the subject's genome.

According to certain embodiments of the invention it is preferable to employ methods that can detect the presence of multiple polymorphic variants (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

The invention provides a database comprising a list of polymorphic sequences stored on a computer-readable medium, wherein the polymorphic sequences occur in a coding or noncoding portion of a gene encoding an EGR molecule or an EGR interacting molecule, or in a genomic region linked to such a gene, and wherein the list is largely or entirely limited to polymorphisms have been identified as useful in performing genetic diagnosis of schizophrenia or susceptibility to schizophrenia, or for performing genetic studies of schizophrenia or susceptibility to schizophrenia.

C. Primers, Probes, Oligonucleotide Arrays, and Kits

The invention provides oligonucleotide probes and primers that can detect polymorphic variants of polymorphisms in a coding or noncoding portion of gene encoding an EGR molecule or an EGR interacting molecule, or polymorphic variants of a polymorphism in a genomic region linked to a coding or noncoding portion of a gene encoding an EGR molecule or an EGR interacting molecule. According to certain embodiments of the invention the presence of a particular polymorphic variant at the polymorphic site is indicative of susceptibility to or diagnostic of schizophrenia. The genes include, but are not limited to, primers that can detect polymorphisms in any of the genes described herein (e.g., genes listed in Table 1 and genes that are targets for regulation by an EGR molecule). In particular, the invention provides oligonucleotide probes and primers that are able to detect polymorphic variants of the polymorphisms in the EGR1, EGR2, EGR3, or EGR4 gene, as defined in Tables 2, 3, 4, and 5.

According to certain embodiments of the invention the allele specific primers and/or probes preferably correspond exactly with the allele to be detected (i.e., they are identical in sequence or perfectly complementary to a portion of DNA that encompasses the polymorphic site, wherein the site contains any of the possible variants), but derivatives thereof are also provided wherein, for example, about 6-8 of the nucleotides at the 3′, terminus correspond with (i.e., are identical in sequence or perfectly complementary to) the allele to be detected and wherein up to 10, such as up to 8, 6, 4, 2 or 1 of the remaining nucleotides may be varied without significantly affecting the properties of the primer or probe.

The invention further provides a set of oligonucleotide primers, wherein the primers terminate adjacent to a polymorphic site in a coding or noncoding portion of gene encoding an EGR molecule or an EGR interacting molecule, or wherein the primers terminate adjacent to a polymorphic site in a genomic region linked to a coding or noncoding portion of a gene encoding an EGR molecule or an EGR interacting molecule. Such primers are useful, for example, in performing fluorescence polarization template-directed dye-terminator incorporation, as described above. In particular, the invention provides oligonucleotide primers that terminate immediately adjacent to the polymorphic sites in the gene encoding EGR1, EGR2, EGR3, or EGR4, as defined in Tables 2, 3, 4, and 5, respectively.

The invention provides, for each of these polymorphisms, a primer that terminates at the nucleotide position immediately adjacent to a polymorphic site on the 3′ side and extends at least 8 and less than 100 nucleotides in the 5′ direction from this site. It is noted that the foregoing includes two classes of primers, having sequences representing both DNA strands. According to certain embodiments of the invention the primer extends at least 10, at least 12, at least 15, or at least 20 nucleotides in the 5′ direction. According to certain embodiments of the invention the primer extends less than 80, less than 60, less than 50, less than 40, less than 30, or less than 30 nucleotides in the 5′direction. The invention further provides primers that terminate and extend similarly for any polymorphic site in a gene encoding a calcineurin subunit or calcineurin interacting molecule, or a polymorphic site in a genomic region linked to such a gene, wherein a polymorphic variant of a polymorphism located at the polymorphic site confers susceptibility to schizophrenia or is indicative of the presence of schizophrenia.

In general, primers and probes may be made using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993. According to certain embodiments of the invention the primer(s) and/or probes are labeled to facilitate detection.

The primers and probes of the invention may be conveniently provided in sets, e.g., sets capable of determining which polymorphic variant(s) is/are present among some or all of the possible polymorphic variants that may exist at a particular polymorphic site. The sets may include allele-specific primers or probes and/or primers that terminate immediately adjacent to a polymorphic site. Multiple sets of primers and/or probes, capable of detecting polymorphic variants at a plurality of polymorphic sites may be provided.

The primers or probes may be provided in the form of a kit for diagnostic and/or research purposes, which may further comprise any of a variety of other components including, but not limited to, appropriate packaging and instructions for use in the methods of the invention, appropriate buffer(s), nucleotides, and/or polymerase(s) such as thermostable polymerases, for example Taq polymerase, other enzymes, positive and negative control samples, negative control primers and/or probes, etc.

The invention further provides oligonucleotide arrays comprising one or more of the inventive probes described above. In particular, the invention provides an oligonucleotide array comprising oligonucleotide probes that are able to detect polymorphic variants of any of the polymorphisms listed in Tables 2, 3, 4, and 5. Such arrays may be provided in the form of kits for diagnostic and/or research purposes. Kits may include any of the components mentioned above, in addition to further components specific for hybridization and processing of oligonucleotide arrays. Appropriate software (i.e., computer-readable instructions stored on a computer-readable medium) for analyzing the results obtained by scanning the arrays may be provided by the invention. Such software may, for example, provide the user with an indication of the genotype of a sample and/or provide an assessment of the degree of susceptibility of the subject to schizophrenia, or an assessment of the likelihood that the subject suffers from schizophrenia.

According to certain embodiments of the invention the kits are manufactured in accordance with good manufacturing practices as required for FDA-approved diagnostic kits.

D. Detection of Alterations in mRNA. According to certain embodiments of the invention alterations or variations in mRNA expression are detected in order to determine whether a subject is susceptible to or suffers from schizophrenia. The expression level (i.e., abundance), expression pattern (e.g., temporal or spatial expression pattern, which includes subcellular localization, cell type specificity), etc., of mRNA encoding a calcineurin subunit or a calcineurin interacting molecule in a sample obtained from a subject is determined and compared with the expression level or expression pattern that would be expected in a sample obtained from a normal subject. mRNA size, processing (e.g., presence of splicing variants, polyadenylation, etc.) may also be compared. According to certain embodiments of the invention the EGR molecule or EGR interacting molecule is one that is encoded by a gene within or linked to a schizophrenia susceptibility locus, or within which a functional mutation causing or contributing to susceptibility or development of schizophrenia may exist.

In general, such detection and/or comparison may be performed using any of a number of suitable methods known in the art including, but not limited to, Northern blotting, cDNA or oligonucleotide array hybridization, in situ hybridization, RNase protection, PCR (e.g., RT-PCR, quantitative PCR) etc. Historical data (e.g., the known expression level, pattern, or size in the normal population) may be used for purposes of the comparison rather than performing the detection method on a control sample.

The invention provides cDNA probes and PCR primers useful for performing the analyses described above, e.g., cDNA probes and PCR primers that specifically hybridize to one or more polymorphic variants. Such probes and/or primers may encompass a polymorphic site and may be perfectly complementary to or identical in sequence to a region encompassing the site, in any of its possible variants. According to certain embodiments of the invention the probes and/or primers are exon-specific, e.g., they hybridize selectively or specifically to variants that either contain or lack a particular exon. Kits for diagnostic and/or research purposes containing, for example, cDNA probes and/or primers as described above, in addition to other components such as those mentioned above, are also provided by the invention.

E. Detection of Alterations in Protein. According to certain embodiments of the invention alterations or variations in protein expression and/or activity are detected in order to determine whether a subject is susceptible to or suffers from schizophrenia. The expression level (i.e., abundance), expression pattern (e.g., temporal or spatial expression pattern, which includes subcellular localization, cell type specificity), size, association with other cellular constituents (e.g., in a complex with DNA), etc., of an EGR molecule or an EGR interacting molecule, in a sample obtained from a subject is determined and compared with the expression level or expression pattern that would be expected in a sample obtained from a normal subject. According to certain embodiments of the invention the EGR molecule or EGR interacting molecule, is one that is encoded by a gene within or linked to a schizophrenia susceptibility locus, or within which a functional mutation causing or contributing to susceptibility or development of schizophrenia may exist.

In general, such detection and/or comparison may be performed using any of a number of suitable methods known in the art including, but not limited to, immunoblotting (Western blotting), immunohistochemistry, ELISA, radioimmunoassay, protein chips (e.g., comprising antibodies to the relevant proteins), etc. Historical data (e.g., the known expression level, activity, expression pattern, or size in the normal population) may be used for purposes of the comparison.

The present invention provides an antibody able to specifically bind to an EGR molecule or an EGR interacting molecule, wherein the subunit or molecule is encoded by a gene within or linked to a schizophrenia susceptibility locus, or within which a functional mutation causing or contributing to susceptibility or development of schizophrenia may exist. In particular, the invention provides an antibody able to specifically bind to a variant of such a an EGR molecule or an EGR interacting molecule, wherein the presence of the variant in a subject is indicative of susceptibility to or presence of schizophrenia. Such antibodies are able to distinguish between EGR molecules or EGR interacting molecules and variants that differ at sites encoded by polymorphic variants.

Generally applicable methods for producing antibodies are well known in the art and are described extensively in references cited above, e.g., Current Protocols in Immunology and Using Antibodies: A Laboratory Manual. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. For example, if a peptide is used the resulting antibody will bind only to the antigenic determinant represented by that peptide. It may be desirable to develop and/or select antibodies that specifically bind to particular regions of the polypeptide, e.g., the extracellular domain. Such specificity may be achieved by immunizing the animal with peptides or polypeptide fragments that correspond to that region. Alternately, a panel of monoclonal antibodies can be screened to identify those that specifically bind to the desired region. As mentioned above, according to certain embodiments of the invention the antibodies specifically bind to antigenic determinants that comprise a region encoded by a polymorphic site. According to certain embodiments of the invention such antibodies are able to distinguish between molecules that differ by a single amino acid. Any of the antibodies described herein may be labeled.

The invention provides any of the foregoing antibodies in panels, e.g., panels of antibodies able to specifically bind to multiple variants of any particular EGR molecule or EGR interacting molecule and panels of antibodies able to specifically bind to multiple variants of a plurality of an EGR molecule or an EGR interacting molecules. The antibodies may be provided in kits, with additional components as mentioned above, including substrates for an enzymatic reaction. The antibodies may be used for research, diagnostic, and/or therapeutic purposes.

In general, preferred antibodies will possess high affinity, e.g., a K_{d} of <200 nM, and preferably, of <100 nM for their target. According to certain embodiments of the invention preferred antibodies do not show significant reactivity with normal tissues, e.g., tissues of key importance such as heart, kidney, brain, liver, bone marrow, colon, breast, prostate, thyroid, gall bladder, lung, adrenals, muscle, nerve fibers, pancreas, skin, etc. Antibodies with low reactivity towards heart, kidney, central and peripheral nervous system tissues and liver are particularly preferred. In the context of reactivity with tissues, the term “significant reactivity”, as used herein, refers to an antibody or antibody fragment, which, when applied to a tissue of interest under conditions suitable for immunohistochemistry, will elicit either no staining or negligible staining, e.g., only a few positive cells scattered among a field of mostly negative cells.

According to certain embodiments of the invention the functional activity of an EGR molecule or an EGR interacting molecule in a sample obtained from a subject is detected and/or measured and is compared with the activity of the EGR molecule or EGR interacting molecule that would be expected in a sample obtained from a normal subject. According to certain embodiments of the invention the EGR molecule or EGR interacting molecule is one that is encoded by a gene within or linked to a schizophrenia susceptibility locus, or within which a functional mutation causing or contributing to susceptibility or development of schizophrenia may exist. It will be appreciated that the particular assay to be employed in detecting and/or measuring the functional activity will depend on the particular molecule being assayed. For example, if the molecule is a kinase or phosphatase, the appropriate assay will be a kinase or phosphatase assay, respectively, using a substrate of the kinase or phosphatase. In the case of a transcription factor such as an EGR molecule, the activity may be ability to activate or repress transcription, which may be measured using an appropriate reporter construct. The activity may be ability to bind to another molecule and/or to inhibit the activity of that other molecule, etc. For example, NAB1 and NAB2 are known to bind to EGR1, EGR2, and EGR3 and to function as transcriptional corepressors or coactivators, depending on the target DNA binding site with which the EGR molecule interacts. In such a case the activity may be binding of NAB1 or NAB2 to an EGR molecule and/or ability of the EGR molecule to activate or inhibit transcription of a reporter containing an appropriate DNA binding site. The activity of a transcription factor to bind DNA (e.g., to specifically bind a DNA target sequence), or the ability of a second molecule to increase or decrease binding affinity of a transcription factor for DNA or for any particular target sequence can also be measured by methods well known in the art, e.g., nuclear footprinting, gel-shift assays, etc.

V. Methods and Reagents for Screening for Compounds Useful in Treating Schizophrenia or Schizophrenia Susceptibility

The invention provides a number of methods and reagents that may be used to screen for compounds useful in treatment of schizophrenia or schizophrenia susceptibilty. It is noted that any of the inventive reagents, methods, and compounds identified according to these methods are not limited to uses related to treatment of schizophrenia or susceptibility to schizophrenia but may be employed for a variety of other purposes. It is also noted that the screens described below are divided into categories for convenience and ease of understanding only, and the classification is not intended to limit the applications of the compounds in any way or place any limitations on their mechanism(s) of action.

A. Screens for Compounds that Modulate (Enhance or Reduce) EGR Activity.

1. Transcription assay. According to one of the inventive methods, DNA transfection, electroporation, etc., is used to express an EGR molecule and, optionally, one or more EGR interacting molecules such as NAB molecules, and to introduce an EGR reporter construct (e.g., a construct comprising a nucleic acid encoding a detectable marker operably linked to an EGR-responsive regulatory element comprising one or more EGR DNA binding sites), into cells (e.g., a cell line) that are suitable hosts for EGR function. Expression of the EGR molecule and EGR interacting molecules may be achieved by transfection of a suitable expression constuct containing coding sequences for the molecules operably linked to promoters active in the particular cell type. Other methods of introducing the EGR molecule and EGR interacting molecules into cells, e.g., microinjection, could also be used. Alternatively, a cell line that expresses one or more EGR molecules and/or NAB molecules can be used as a host, and any desired components not expressed endogenously provided, e.g., by DNA transfection. In general, many commonly available cell lines are suitable hosts, e.g., CV-1 cells, COS cells, T cells, NIH3T3 cells. According to certain embodiments of the invention a cell line exhibiting features similar to those in which a therapeutic effect is desired may be used, e.g., a neural or glial cell line.

According to certain embodiments of the invention it may be preferable to avoid use of cells that naturally express NAB1 or NAB2. In certain embodiments of the invention expression of an undesired molecule is reduced or eliminated by RNAi, e.g., by introducing an siRNA or shRNA targeted to the molecule into the cell. For example, in a screen to detect activators or inhibitors of a particular EGR molecule, it may be desirable to minimize expression of other EGR molecules. For such purposes it is preferable to select a target region of the mRNA whose levels are to be reduced by RNAi that lacks significant homology to mRNA encoding the EGR molecule for which activators or inhibitors are sought in the screen. In a screen to identify molecules that enhance or disrupt binding between an EGR molecule and a specific EGR binding molecule such as NAB1 or NAB2, it may be desirable to minimize expression of other EGR binding molecules.

The EGR-responsive regulatory element may contain one or more EGR consensus DNA binding sites or any other EGR DNA binding site(s) (either identical to a naturally occurring EGR DNA binding site or artificial in the sense that it is not found in nature), or combination thereof. By appropriate selection of the sequence and number of EGR DNA binding sites, it is possible to determine whether NAB proteins will repress or activate transcription of the reporter gene, which is of use in performing certain of the inventive screens. For example, if a system in which presence of NAB proteins activates transcription is desired, a regulatory element resembling that in the TGF-β gene regulatory region can be used. If a system in which presence of NAB proteins represses EGR-mediated transcription is desired, a regulatory element resembling that in the LHβ gene regulatory region can be used. The ability of an EGR molecule to activate transcription and/or of an NAB molecule to repress or activate EGR-mediated transcription can readily be determined. In general, a wide variety of reporter genes encoding detectable markers can be used. Suitable markers include β-galactosidase (encoded by lacZ), green fluorescent protein (GFP) and numerous variants thereof, luciferase, etc. A number of EGR reporter constructs are known in the art. See, e.g., 68-70, and references therein). It may be desirable to employ a marker whose expression is readily quantifiable.

Cells expressing all desired components (EGR molecule, EGR reporter, and optionally EGR interacting molecule(s)), may be treated with a growth factor such as nerve growth factor (NGF) to stimulate EGR transcriptional activity, which may be measured by assessing activity of the reporter construct, e.g., luciferase in the case of a reporter comprising a luciferase gene operably linked to an EGR target DNA sequence. Comparison of reporter activity in the absence or presence of compounds (e.g., a member of a combinatorial library, natural product collection, etc.), is used to identify compounds that yield altered (e.g., increased or decreased) EGR-mediated transcription. Among these compounds will be those that increase or decrease EGR activity directly, e.g., through direct binding to an EGR molecule, or via other interactions, e.g., by binding to an NAB molecule or EGR target DNA sequence. To confirm EGR specificity, the same assay can be performed in the absence of one or more EGR molecules, which should reduce the effect, or with elevated levels of the EGR molecule, which should result in an enhanced effect. This may entail using cell lines deficient for particular EGR molecules or EGR interacting molecules, or reducing expression of one or more EGR molecules or EGR interacting molecules by RNAi during the assay.

Thus the invention provides a method for identifying a candidate compound for treatment of schizophrenia or susceptibility to schizophrenia comprising steps of: (i) providing a biological system containing an EGR molecule and an EGR reporter; (ii) contacting the biological system with a compound; (iii) comparing the transcriptional response of the reporter in the presence of the compound with the response or expected response in the absence of the compound. If the transcriptional response in the presence of the compound is different from (e.g., greater or less than) the transcriptional response that occurs or would be expected in the absence of the compound, the compound is identified as a modulator of EGR activity and a candidate compound for treatment of schizophrenia or susceptibility to schizophrenia. By “biological system” is meant any vessel, well, or container in which biomolecules (e.g., nucleic acids, polypeptides, polysaccharides, lipids, etc.) are placed; a cell or population of cells; a tissue; an organism, etc. Typically the biological system is a cell or population of cells, but the method can also be performed in a vessel using purified or recombinant proteins. In general, such an assay can identify compounds that affect EGR expression or functional activity by any of a number of different mechansims. For example, such compounds may (i) interfere with binding of an EGR molecule to an EGR DNA binding site (e.g., either by themselves binding to the site or binding to the EGR molecule); (ii) disrupting or enhancing binding between an EGR molecule and an EGR interacting molecule; (iii) activating or inhibiting an EGR interacting molecule that regulates EGR function by a mechanism other than binding (e.g., by phosphorylation/dephosphorylation, influencing localization, transducing an extracellular signal, etc.).

The assay can be used in combination with transfection or addition of EGR interacting proteins such as NAB1 or NAB2 to screen for compounds that inhibit or activate the EGR molecule by interfering with or enhancing binding between the EGR molecule and an EGR interacting molecule. Such compounds may, for example, bind to the R1 domain and thereby prevent binding of an NAB protein. As mentioned above, depending upon the particular DNA target sequence present in the reporter, EGR interacting molecules such as NAB1 or NAB2 are expected to either repress (in most cases) or activate (in certain cases) transcriptional activity of EGR molecules with which they interact. In particular, if the EGR reporter contains an EGR responsive regulatory element at which NAB proteins act as repressors, then a molecule that disrupts binding of an NAB protein and an EGR protein will increase reporter activity, and a molecule that enhances binding of an NAB protein and an EGR protein will decrease reporter activity. Conversely, if the EGR reporter contains an EGR responsive regulatory element at which NAB proteins act as activators, then a molecule that disrupts binding of an NAB protein and an EGR protein will decrease reporter activity, and a molecule that enhances binding of an NAB protein and an EGR protein will increase reporter activity. Thus the screens could identify both inhibitory and activating compounds. In any of the foregoing screens, specificity could be determined by repeating the assay in absence of transfection of the gene encoding the interacting molecule or by using RNAi to inhibit expression of the interating molecule. The identified compound can then be tested in an animal model for schizophrenia (see Example 5 and section E, below).

In certain embodiments of the invention the screening methods that utilize EGR molecules, EGR interacting molecules, and/or other cellular molecules can utilize a polymorphic variant of such molecule, e.g., a variant that is associated with schizophrenia or schizophrenia susceptibility. It is also noted that the screening methods described herein that utilize EGR molecules, EGR interacting molecules, etc., need not be performed with molecules that are identical to the versions thereof that exist in nature. Instead, the molecules may have one or more nucleotide or amino acid substitutions, deletions, and/or additions relative to their naturally occurring counterparts. In certain embodiments of the invention an amino acid substitution or deletion in an EGR molecule reduces or eliminates interaction of the molecule with an NAB protein, and such molecule is used. In certain embodiments of the invention an amino acid substitution is a conservative substitution, i.e., a substitution in which an amino acid residue is replaced with an amino acid residue having a similar side chain. Families of amino acid residues having similar side chains are known. For example, such families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In certain embodiments of the invention molecules that are not identical to naturally occurring versions but that are substantially identical (e.g., at least 70% identical, preferably at least 80%, at least 90%, at least 95%, or at least 99% identical) are used. For example, the molecule may have between 1 and 10 nucleotide or amino acid substitutions, additions, or deletions alterations respect to a naturally occurring molecule.

2. Additional screens for molecules that block binding of EGR proteins and EGR binding proteins. Standard yeast 2 and/or 3 hybrid assays can be used to screen for molecules that block binding of EGR binding proteins such as NAB1 or NAB2 to an EGR protein, e.g., EGR1, EGR2, or EGR3, or that block binding of an EGR binding protein such as NAB1 or NAB2 to a fragment of EGR1, EGR2, or EGR3, such as the R1 domain. Phage display can be used to select peptides that bind to EGR molecules, EGR interacting molecules, or fragments thereof such as the R1, NCD1, or NCD2 domains. In vitro binding assays can also be used to identify compounds, e.g., small molecules, that specifically bind to an EGR molecule or portion thereof, or to an EGR interacting molecule or portion thereof. The molecule or portion thereof can be expressed using recombinant DNA techniques, chemically synthesized, or purified from cells. According to certain embodiments of the invention, bioluminescence resonance energy transfer screens with recombinant, fluorescently tagged EGR and NAB proteins (Boute, N., et al., Trends in Pharmacol Sci (2002) 23 (8): 351-4) are used.

Direct in vitro binding assays, e.g., using labeled compounds, can also be used to identify compounds that bind to EGR molecules or EGR interacting molecules, independent of their ability to disrupt binding of EGR molecules with EGR binding proteins. Compounds may be tested in vitro or in vivo for their ability to inhibit or enhance binding of an EGR molecule to an inhibitory or activating protein. For example, the ability of a candidate compound to inhibit binding of an EGR molecule to an inhibitory or activating protein may be tested in vitro using purified (e.g., recombinant) proteins and/or extracts from cells expressing one or more of these components. Components may be mixed in the absence or presence of the candidate compound, and complexes containing the EGR molecule can be isolated and assayed to determine whether they contain the inhibitory or activating protein. Appropriate isolation and detection methods (e.g., immunoprecipitation, Western blot, ELISA) can be employed. The ability of a candidate compound to inhibit binding of an EGR molecule to an inhibitory or activating protein may similarly be assayed in intact cells using, e.g., co-immunoprecipitation. Cells may naturally express the components or be engineered to do so. The identified compound can then be tested in a cell-based assay such as those described above and/or administered to an animal model for schizophrenia (see Example 5).

In any methods employing recombinant proteins it may be desirable to use proteins that include a tag, e.g., a GST tag, FLAG tag, HA epitope tag, etc. Construction of appropriate expression vectors and their introduction into cells can be performed using standard methods, e.g., as described in Current Protocols in Molecular Biology. It may be desirable to engineer EGR proteins or one or more EGR interacting molecules to include a readily detectable marker, e.g., a fluorescent or luminescent marker such as GFP. Such readily detectable proteins may be useful to study the effect of compounds on the subcellular localization of the protein and/or its binding to DNA, etc. Any of the various compound identification and screening methods described above may be employed in a high throughput format or may readily be modified for high throughput screening.

B. Molecular Drug Design. The invention provides methods for rational drug design based on molecular modeling for identification of candidate compounds for treatment of schizophrenia or schizophrenia susceptibility, e.g., using the three-dimensional structure (crystal structure, NMR solution structure, etc.) of an EGR molecule or an EGR interacting molecule such as NAB1 or NAB2. Such methods may be particularly useful, e.g., to identify compounds that may interfere with binding between an EGR molecule such as EGR1, EGR2, or EGR3, and NAB1 or NAB2.

Structural information may also be used to guide selection of appropriate compound libraries for screening. Thus the invention provides a method for identifying a candidate compound for treatment of schizophrenia or schizophrenia susceptibility comprising steps of: (i) providing a molecular structure of an EGR molecule or a complex containing an EGR molecule; (ii) identifying a structure that is expected to bind to the EGR molecule or to prevent binding of an EGR molecule to an EGR interacting molecule; and (iii) selecting a compound having such a structure as a candidate compound for treatment of schizophrenia or schizophrenia susceptibility. The identified compound can then be administered to an animal model for schizophrenia (see Example 5).

C. Screens for Additional EGR Interacting Molecules. While not wishing to be bound by any theory, the inventors' discovery that the four EGR genes are located in genomic regions associated with schizophrenia susceptibility (see Example 1) suggests that EGR molecules and EGR interacting molecules, including potential target genes of EGR-regulated genes, could be involved with schizophrenia pathogenesis. Therefore, EGR interacting molecules, and EGR regulated genes, are attractive molecular targets for development of diagnostic and/or therapeutic agents. Compounds that interact with EGR molecules and EGR regulated gene products are attractive candidate compounds for treatment of schizophrenia or susceptibility to schizophrenia. Additional EGR interacting proteins can be identified using standard two or three hybrid methodology (e.g., in yeast or mammalian cells, etc., using an EGR molecule or portion thereof as bait. In addition, EGR interacting proteins can be identified by standard biochemical means, for example, subjecting cellular extracts to EGR affinity column chromatography using either a full length EGR protein or a portion thereof.

A variety of approaches may be used to identify additional EGR regulated genes. The human genome draft sequence can be searched for DNA sequences that are identical to or resemble the EGR consensus DNA binding sequence or EGR binding sequences found in genes known to be regulated by EGR proteins. Alterately, fragments of DNA that bind to recombinantly expressed EGR molecules can be isolated. Reporter assays can be used to analyze the ability of EGR proteins to activate or repress transcription from a candidate EGR response element. Gel shift assays, nuclease protection assays, DNase I footprinting assays, and other methods known in the art can be used to confirm that a particular regulatory region is bound by an EGR molecule and/or to map the binding sites. Genes whose regulatory regions contain one or more EGR DNA binding sites are candidate EGR regulated genes.

Another approach is to perform differential screening. For example, mRNA isolated from cells that have been exposed to a growth factor or other stimulus that activates EGR can be compared with mRNA isolated from cells that have not been exposed. The comparison may conveniently be performed using cDNA microarrays. Other forms of screening (e.g., subtractive hybridization), serial analysis of gene expresson (SAGE) could also be used (Velculescu, V. E., Zhang, L., Vogelstein, B., and Kinzler, K. W. (1995). Serial Analysis Of Gene Expression. Science 270, 484-487). Genes whose mRNAs whose expression is altered (either upregulated or down-regulated) under conditions in which EGR activity is stimulated are candidate EGR regulated genes. Whether a gene is upregulated or downregulated will depend, in general, upon the specific EGR binding site and/or presence of EGR binding molecules such as NAB proteins in the system. Quantitative RT-PCR can be performed to confirm the difference in expression.

RNAi can be used to inhibit expression of an EGR molecule or EGR interacting molecule such as an NAB protein. mRNA isolated from cells in which an EGR molecule is inhibited can be compared with mRNA isolated from cells in which the EGR molecule is not inhibited. Again, the comparison can be performed using cDNA microarrays or other methods. Genes whose mRNAs whose expression is altered (either upregulated or downregulated) under conditions in which EGR activity is inhibited are candidate EGR regulated genes. Searching the upstream region of such genes for EGR consensus or nonconsensus binding sites can be performed to reduce the number of candidate genes. Reporter assays and in vitro binding assays can be used to confirm the identity of a candidate gene as being an actual EGR regulated gene. The invention therefore provides a number of methods for the identification of additional EGR regulated genes.

The position of any such gene identified as an EGR regulated gene or as encoding an EGR interacting molecule of any type may be tested according to the methods described herein to determine whether it is coincident with a previously identified schizophrenia locus and/or genetic linkage studies can be performed to determine whether an association with schizophrenia susceptibility exists. If such coincidence and/or genetic linkage is found, the gene is a candidate for the diagnosis and/or treatment of schizophrenia in accordance with the methods described herein. Polymorphisms in the gene can be identified using the methods described herein (e.g., searching databases, sequencing, etc.) The invention thus provides a method of identifying a target for diagnosis and/or treatment of schizophrenia or schizophrenia susceptibility comprising steps of: (a) identifying a gene encoding an EGR interacting molecule; and (b) determining whether the location of the gene is coincident with a known schizophrenia susceptibility locus or performing a genetic study, e.g. a linkage study, to determine whether a polymorphism in the gene is associated with schizophrenia or schizophrenia susceptibility. The gene can be an EGR regulated gene or a gene that encodes a molecule that binds to an EGR protein or regulates EGR activity or expression, etc.

D. Compounds for Screening. Compounds suitable for use in any of the compound identification methods described above (or other methods) include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd CD, Leblanc C, Whittaker M, Prog Med Chem 36: 91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. See also U.S. Patent Application No. 6,448,443 and PCT publication WO9964379.

In general, compounds may be dissolved in any appropriate solvent, preferably a solvent that does not exert deleterious effects on the growth of cells, if the screen involves cells. A range of concentrations of the compound may be tested for the desired effect. As is well known to one of ordinary skill in the art, virtually any compound can have deleterious effects on an organism if present at sufficiently high concentration. Preferred compounds exert their effects at concentrations practical for administration as therapeutic agents (e.g., at concentrations that do not cause unacceptable adverse effects in the subject being treated). Screens can be performed, for example, at relatively low concentrations such as <0.1 μg/ml, at higher concentrations such as 0.1 to 1 μg/ml, 1 to 100 μg/ml, or at still higher concentrations, e.g., up to 1 mg/ml. In general, one of ordinary skill in the art will be able to select appropriate concentration ranges for testing, and the foregoing examples are not intended to be limiting.

According to one approach, polypeptides having a sequence comprising the sequence of the R1 domain, which is found in EGR1, EGR2, or EGR3, or a portion of the R1 domain are used. An R1 domain from either a human or non-human (e.g., mouse) EGR1, EGR2, or EGR3 could be used. The R1 domain has been shown to act as a dominant negative, reducing or preventing the interaction between EGR molecules containing an R1 domain and NAB proteins that would otherwise inhibit (in most cases) or activate (in some cases) the EGR protein with respect to a particular EGR regulated gene (71). Fragments of R1 can be tested (e.g., using reporter assays as described above) to identify small polypeptides capable of modulating EGR activity by inhibiting the interaction of an EGR protein and an NAB protein. Small molecule peptidomimetics are designed based on the structure of such a polypeptide using methods known in the art and are tested for their ability to disrupt EGR-NAB interactions. It is possible that such screens will also identify molecules that stabilize the EGR-NAB interaction. These peptidomimetics can be used to generate a combinatorial library of molecules suitable for screening.

E. Screens for Effectiveness of Candidate Compounds in Animal Models and Humans

Candidate compounds identified using any of the screening methods described herein (or other suitable methods) can be tested in any appropriate animal model for schizophrenia including both genetic and pharmacological models. For example, such compounds can be tested in a CNB forebrain-specific knockout mouse (19) and/or in any of the models described in Gainetdinov, et al., “Genetic animal models: focus on schizophrenia”, Trends in Neurosciences, Vol. 24, No. 9, September 2001. Such models include various selected or developed (e.g., using gene targeting technology) strains of mice (e.g., mice having mutations or deletions in various components of neurotransmitter systems such as the NMDA receptor, dopamine transporter, etc.). Alternately, animal models may be obtained by exposing the animals to appropriate compounds such as PCP, etc., that result in development of symptoms suggestive of schizophrenia.

When testing compounds in animal models, it may be preferred to use an animal model that does not contain a mutation or deletion in the expected target of the compound (although such animal models may usefully be employed as controls for specificity of the compound since if the compound is similarly effective in such animal models it is most likely acting via a mechanism that does not involve interaction with the expected target). Candidate compounds can also be tested in human subjects suffering from schizophrenia or a related condition or susceptibility thereto.

In general, such tests for efficacy involve administering the candidate compound to the subject (whether animal or human) and observing the subject to determine whether administration of the compound results in amelioration in or reduction of any sign or symptom of schizophrenia (or results in a decreased incidence of developing schizophrenia). Any of the phenotypes characteristic of animal models of schizophrenia (i.e., phenotypes suggestive of schizophrenia) may be assessed, including, but not limited to, those activities and behaviors described in Example 5. In particular, phenotypes such as (1) locomotor activity; (2) stereotyped behavior; (3) exploratory behavior towards inanimate objects; and (4) anxiety-like behavior can be assessed. Increases in these activities and behaviors are considered to indicate disturbances in cognitive functioning corresponding to disturbances found in human subjects suffering from schizophrenia and/or related conditions. Additional aspects of behavior and/or activity such as (1) social interaction; (2) prepulse inhibition; (3) latent inhibition; and (4) nesting behavior. Impairment or abnormality in most or all of these abnormalities are considered to indicate disturbances in cognitive functioning corresponding to disturbances found in human subjects suffering from schizophrenia and/or related conditions. Many of them are also found in a variety of currently available genetic mouse models and/or in mice treated with pharmacological compounds (e.g., cocaine, PCP), known to induce schizophrenia-like symptoms in human subjects.

In humans, any of the parameters used in the diagnosis and/or assessment of patients suffering from or suspected of suffering from schizophrenia or a related condition may be assessed. Methodology for performing clinical trials of candidate therapeutic agents for schizophrenia in humans is well established. According to certain embodiments of the invention human subjects for the clinical trial are selected by identifying subjects at risk of or suffering from schizophrenia using any of the inventive methods described herein. For example, subjects may be selected by detecting a polymorphic variant of a polymorphism in a coding or noncoding portion of a gene encoding an EGR molecule, an EGR interacting molecule, a calcineurin subunit, or a calcineurin interacting molecule, or by detecting a polymorphic variant of a polymorphism in a genomic region linked to such a gene, in a sample obtained from the subject. According to certain embodiments of the invention a group of subjects selected using any of the inventive methods is compared with a group of subjects selected using any other diagnostic criterion.

Thus the invention provides a method for identifying a candidate compound for treatment of schizophrenia comprising steps of: (i) providing a subject or subjects at risk of or exhibiting one or more phenotypes suggestive of schizophrenia, wherein the subject or subjects have an alteration in expression of at least EGR molecule or EGR interacting molecule; (ii) administering the candidate compound to the subject or subjects; (iii) comparing severity or incidence of the phenotype in the subject or subjects to severity or incidence of the phenotype in a subject or subjects to which the compound is not administered. Typically the method will be performed using groups of animals. If the phenotype appears less severe or occurs at reduced frequency in the subject(s) to which the compound is administered, the compound is identified a candidate compound for the treatment of schizophrenia and/or schizophrenia susceptibility (although of course this may be confirmed using additional methods).

The invention further provides a method of identifying a candidate compound for treatment or prevention of schizophrenia or schizophrenia susceptibility comprising: (a) administering a compound that increases of decreases expression or activity of an EGR molecule or an EGR interacting molecule to an animal that constitutes an animal model for schizophrenia; (b) evaluating at least one phenotypic parameter in the animal, wherein in the absence of the compound the animal has an alteration in the phenotypic parameter relative to its normal value that is suggestive of schizophrenia; and (c) identifying the compound as a candidate compound for treatment or prevention of schizophrenia or schizophrenia susceptibility if administration of the compound restores the phenotypic parameter towards a more normal value. The value of the parameter can be compared with its value in subjects that do not receive the compound. According to certain embodiments of the invention the subject that receive the compound and those that do not receive the compound (i.e., controls) may be genetically similar or identical animals. (It is noted that historical controls can be used.) The effect of the compound can also be evaluated in wild type mice and/or in human subjects. The animal model may or may not have an alteration in a gene encoding an EGR molecule or an EGR interacting molecule.

The compound may be, for example, an RNAi-inducing agent such as an siRNA targeted to a transcript encoding an EGR molecule or an EGR interacting molecule (which would reduce expression of the EGR molecule or EGR interacting molecule), an RNAi-inducing agent such as an siRNA targeted to an NAB protein (which would reduce expression of the NAB protein and thereby increase (in most cases) or decrease (in some cases) EGR functional activity with respect to transcription of a particular EGR regulated gene. The compound may be a polypeptide having a sequence comprising the sequence of the R1 domain of EGR1, EGR2, or EGR3, or a portion of the R1 domain. As mentioned above, the R1 domain has been shown to act as a dominant negative, blocking the interaction between EGR molecules containing an R1 domain and NAB proteins that would otherwise inhibit (in most cases) or activate (in some cases) the EGR protein with respect to a particular EGR regulated gene. The compound may be a molecule identified through any of the screening approaches described above. In general, compounds can be delivered using any available route including intravenous administration, oral administration, focal delivery (e.g., injection into a target site such as the brain, intrathecal delivery), inhalationally, transdermally, etc.

F. Animal Models for Efficacy Screens of Candidate Compounds. The invention provides a transgenic animal, e.g., a mouse, expressing an altered form of an EGR molecule or an EGR interacting molecule. The invention further provides a transgenic animal that overexpresses an EGR molecule or an EGR interacting molecule. The invention provides an EGR murine hypomorph, wherein the hypomorphic locus can be any gene encoding an EGR molecule or an EGR interacting molecule. Preferably the mouse displays one or more phenotypes suggestive of schizophrenia.

By “hypomorph” is meant an animal that expresses a given gene at less than wild type levels but at greater levels than would result from complete deletion of the gene (or other complete elimination of expression). For example, a hypomorph may express a gene at less than 50%, less than 25%, less than 10%, less than 5%, less than 1%, or an even lower percentage, but greater than 0, of the wild type level of expression. Hypomorphs expressing between 10% and 30% of the wild type level of expression may be preferred. Hypomorphic mice may be particularly useful when complete deletion or inactivation of the gene results in embryonic lethality or severe defects that prevent or impede assessment of phenotypes of interest (e.g., phenotypes suggestive of schizophrenia). Hypomorphic mice may be created, for example, by “knock in” of promoters such as the PGK promoter as described in [25]. This promoter has been shown to severely repress transcription at targeted loci, and this method has been used to create a mouse NMDA receptor hypomorph [25]. In general, the term “hypomorph” does not refer to tissue-specific or regional knockouts. However, the term includes tissue or region-specific reductions in expression.

The invention further provides mice having tissue restricted expression of any gene encoding an EGR molecule or an EGR interacting molecule. In particular, the invention provides mice lacking or having reduced expression of a gene encoding an EGR molecule or an EGR interacting molecule in one or more nervous system regions, e.g., in one or more regions of the brain.

In general, mice that over-express or under-express any of the above-mentioned genes may be generated according to a variety of conventional, recently developed, or emerging transgenic or knockout techniques. Such techniques may include use of cell or tissue specific regulatory elements, inducible systems, etc. See, e.g., Kwan, K., “Conditional alleles in mice: practical considerations for tissue-specific knockouts.” Genesis, 32 (2): 49-62, 2002; Lewandowski, M., “Conditional control of gene expression in the mouse”, Nat. Rev. Genet., 2 (10): 743-55, 2001; Bockamp, E., et al., Physiol Genomics 11 (3): 115-32 (2002).

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. Thus “knockout” animals are included. A transgene can, but need not, replace an endogenous gene. A transgene can direct the expression of an encoded product in one or more cell types or tissues of the transgenic animal.

According to certain embodiments of the invention murine hypomorphs or effective null mutants are generated by expressing an siRNA, shRNA, mRNA, or other RNAi-inducing agent targeted to an EGR molecule or targeted to an EGR interacting molecule in the mouse (which may be done in a tissue or cell type specific manner, or in an inducible manner if desired.) According to certain embodiments of the invention such expression is achieved using lentiviral vectors as described, for example, in Rubinson, D. A., et al., Nature Genet. 33: 401-406 (2003); An, D. S., Hum. Gene Ther., 14: 1207-1212 (2003) or in other references cited in Dorsett, Y. and Tuschl, T., Nature Reviews Drug Discovery, 3: 318-329 (2004), which are herein incorporated by reference. Such mice can be crossed to generate compound homo or heterozygotes. The invention further provides mice generated by crossing any of the inventive mice described above either with other members of this group or with mice of any different genotype including, but not limited to, mice used as models for schizophrenia (e.g., mice having mutations in the gene encoding a subunit of the NMDA receptor, dopamine transporter, calcineurin subunit, calcineurin interacting molecule, etc.), and mice expressing a recombinase such as Cre in a tissue-specific manner.

The invention provides transgenic animals that expresses a variant of an EGR molecule or an EGR interacting molecule, wherein the variant occurs at a homolous location to the location of a variant in a homologous human protein, wherein the variant is associated with schizophrenia or susceptibility to schizophrenia. The human variant is encoded by a gene having a polymorphic variant associated with schizophrenia or susceptibility to schizophrenia.

Although described primarily with reference to mice, the invention is not limited to such animals but also includes any other animal in which genetically engineered variants can be made including, but not limited to, rats, sheep, pigs, goats, bovine animals, and, possibly, primates. In particular, it is noted that siRNA mediated gene silencing has been demonstrated in transgenic rats (Hasuwa, H., et al., FEBS Lett 2002 Dec. 4; 532 (1-2): 227-30).

Any of the animals described above may be used to screen for efficacy of candidate compounds for treatment of schizophrenia or susceptibility to schizophrenia. A mouse hypomorphic for any particular protein may be particularly useful for testing compounds designed to enhance the activity of that protein. These mice can be used for both biochemical and behavioral assays to validate candidate compounds.

VI. Compounds and Methods for Treatment of Schizophrenia or Schizophrenia Susceptibility

A. Compounds and Methods of Use. The present invention provides compounds identified according to any of the inventive compound identification methods described above. According to certain embodiments of the invention preferred compounds exhibit the ability to cross the blood-brain barrier, so that a therapeutically effective concentration in the central nervous system may be achieved. Candidate compounds, e.g., compounds identified using in vitro methods may be appropriately modified, e.g., by conjugation with a lipophilic moiety or by any of various methods known in the art in order to enhance their ability to cross the blood-brain barrier. According to certain embodiments of the invention compound libraries, e.g., combinatorial libraries, are synthesized using an appropriate starting compound and/or substituents, so as to increase the likelihood that the compound will cross the blood-brain barrier.

In general, any of the compounds identified as described above may be further optimized to reduce or eliminate undesirable properties and/or to increase or enhance desirable properties. For example, compounds may be modified to increase solubility, increase absorbability, or otherwise enhance bioavailability. Such compounds may be useful as therapies and/or as lead compounds for the design or selection of further compounds. The invention thus provides derivatives of compounds identified according to the screening methods above, e.g., derivatives that display enhanced bioavailability, enhanced ability to cross the blood-brain barrier, improved safety profile, etc.

It is noted that any of the compounds identified according to the inventive methods described above may have a number of additional uses, both for research and therapeutic purposes, and their identification as candidate compounds for use in treatment of schizophrenia or schizophrenia susceptibility is not intended to limit their applications in any way.

The invention provides methods of treating schizophrenia or susceptibility to schizophrenia comprising steps of (i) providing a subject at risk of or suffering from schizophrenia; and (ii) administering a compound identified according to any of the inventive methods described above to the subject. The compound is preferably adminstered in an effective amount for the treatment or prevention of schizophrenia or schizophrenia susceptibility.

The invention provides a method for treating schizophrenia or susceptibility to schizophrenia comprising: (i) providing a subject at risk of or suffering from schizophrenia; and (ii) administering a compound that modulates activity or abundance of an EGR molecule or an EGR interacting molecule to the subject. According to various embodiments of the invention the compound enhances activity or abundance of the EGR molecule or EGR interacting molecule. According to certain other embodiments of the invention the compound reduces activity or abundance of the EGR molecule or EGR interacting molecule. According to certain embodiments of the invention the compound modulates (e.g., enhances or reduces) activity of the EGR molecule or EGR interacting molecule. According to certain embodiments of the invention the EGR molecule or EGR interacting molecule is selected from the group consisting of: EGR1, EGR2, EGR3, EGR4, NAB 1, and NAB2. Appropriate compounds include, but are not limited to, those identified according to any of the inventive compound screening methods described above.

B. Gene Therapy. The invention also provides methods of treating schizophrenia or susceptibility to schizophrenia using gene therapy, wherein a calcineurin subunit or calcineurin interacting molecule (including altered versions of such subunits or molecules) is expressed in cells of a subject. The EGR molecule or EGR interacting molecule may be any of those listed in Table 1, or others. Alternately, according to certain embodiments of the invention an inhibitory siRNA, shRNA, mRNA, or other RNAi-inducing agent targeted to the EGR molecule or EGR interacting molecule is expressed so as to reduce or eliminate endogenous expression of the subunit or molecule. Other gene therapy based methods of reducing expression of these molecules may also be used. Methods for modulating the transcription of genes encoding EGR molecules or EGR interacting molecules are also within the scope of the invention. See, e.g., U.S. Pat. No. 6,326,166. Methods and vectors for gene therapy are known in the art. In general, gene therapy vectors include retroviruses, lentiviruses, adenoviruses, adeno-associated viruses, and a number of non-viral vectors. According to the inventive methods a nucleic acid encoding the desired subunit or molecule is introduced into a gene therapy vector under control of appropriate regulatory elements. Such regulatory elements may be selected to achieve inducible or constitutive expression in a cell type or tissue of choice or throughout the body.

Gene therapy protocols may involve administering an effective amount of a gene therapy vector capable of directing expression of a calcineurin subunit or calcineurin interacting molecule or inhibitory siRNA to a subject either before, substantially contemporaneously, with, or after influenza virus infection. Another approach that may be used alternatively or in combination with the foregoing is to isolate a population of cells, e.g., stem cells or immune system cells from a subject, optionally expand the cells in tissue culture, and administer a gene therapy vector capable of directing expression of a calcineurin subunit or calcineurin interacting molecule or an inhibitory siRNA to the cells in vitro. The cells may then be returned to the subject. Optionally, cells expressing the calcineurin subunit or calcineurin interacting molecule siRNA can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

VII. Additional Methods, Reagents, and Compounds

The invention provides a number of additional methods, reagents, and compounds that may be used either for the treatment of schizophrenia or schizophrenia susceptibility, the development of treatments for schizophrenia or schizophrenia susceptibility, the practice of the other inventive methods described herein, or for a variety of other purposes.

A. RNAi-Inducing Agents. RNA interference (RNAi) is a mechanism of post-transcriptional gene silencing mediated by double-stranded RNA (dsRNA), which is distinct from antisense and ribozyme-based approaches. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus typically mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, in general, the presence of siRNA in mammalian cells, results in sequence-specific gene silencing, preferably without inducing the interferon response or, if such response is induced, without inducing it to levels resulting in unacceptable side effects.

In general, a short, interfering RNA (siRNA) comprises an RNA duplex that is preferably approximately 19 basepairs long and optionally further comprises one or two single-stranded overhangs, e.g., 3′ overhangs. The duplex region may, in general, range from about 15 to about 29 nucleotides in length, and for some purposes duplexes having lengths of greater than 19 base pairs are preferred. An siRNA generally comprises two RNA strands hybridized together, one of which is sense and the other of which is antisense (i.e., complementary) with respect to a target mRNA. siRNAs may include one or more free strand ends, which may include phosphate and/or hydroxyl groups, e.g., 5′ phosphate groups. siRNAs include a portion capable of hybridizing with a target transcript. One strand (the antisense strand) of the siRNA (or, the antisense strand of the self-complementary portion of an shRNA—see below) is typically precisely complementary with a region of the target transcript, meaning that the siRNA or shRNA antisense strand hybridizes to the target transcript without a single mismatch. However, perfect complementarity is generally not required. In certain embodiments of the invention in which perfect complementarity is not achieved, it may be preferred that any mismatches be located at or near the siRNA termini.
siRNAs have been shown to downregulate gene expression when transferred into mammalian cells by such methods as transfection, electroporation, or microinjection, or when expressed in cells via any of a variety of plasmid-based approaches (e.g., as shRNAs that are processed intracellularly to produce siRNAs). RNA interference using siRNA and other RNA-inducing agents is reviewed in, e.g., Tuschl, T., Nat. Biotechnol., 20: 446-448, May 2002. See also Yu, J., et al., Proc. Natl. Acad. Sci., 99 (9), 6047-6052 (2002); Sui, G., et al., Proc. Natl. Acad. Sci., 99 (8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Dorsett, Y, and Tuschl, T., referenced above, the teachings of which are relevant to and supplement the description herein. A number of variations in structure, length, number of mismatches, size of loop, identity of nucleotides in overhangs, etc., are consistent with effective RNAi-triggered gene silencing and it is generally possible to identify one or more effective siRNA sequence able to silence expression of any particular target by at least 80% using a trial and error approach. In addition, a number of publicly available or proprietary algorithms are available for selection of siRNA sequences likely to mediate silencing effectively. See, for example, the Whitehead Institute siRNA Selection Program available at the web site having URL http://jura.wi.mit.edu/siRNAext/.

Molecules referred to as short hairpin RNAs (shRNAs) generally comprise a single self-complementary RNA strand that includes sense and anti-sense (with respect to the sequence of a target mRNA) portions that hybridize to one another to generate a double-stranded (duplex) structure that can be processed intracellularly to produce one or more siRNAs. The complementary portions are typically separated by a non self-complementary region that forms a loop so that the overall structure resembles a hairpin containing a stem, a loop, and optionally an overhang, preferably a 3′ overhang. The stem may be approximately 19 nucleotides, the loop about 1-20, more preferably about 4-10, and most preferably about 6-8 nt long and/or the overhang about 1-20, and more preferably about 2-15 nt long. In certain embodiments of the invention the stem ranges from 15 nucleotides up to 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues. The sequence of the antisense and sense portions of the shRNA are chosen as for siRNAs.
siRNAs as described above trigger degradation of mRNAs to which they are targeted via a DICER-dependent mechanism, thereby also reducing the rate of protein synthesis. In addition to siRNAs that act via this pathway, certain RNAs, e.g., RNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that naturally occurring mRNAs are derived from larger hairpin-like precursors known as small temporal RNAs (stRNAs) or mRNA precursors, which are typically approximately 70 nt long with an approximately 4-15 nt loop. (See Grishok, A., et al., Cell 106, 23-24, 2001; Hutvagner, G., et al., Science, 293, 834-838, 2001; Ketting, R., et al., Genes Dev., 15, 2654-2659). Endogenous RNAs of this type have been identified in a number of organisms including mammals, suggesting that this mechanism of post-transcriptional gene silencing may be widespread (Lagos-Quintana, M. et al., Science, 294, 853-858, 2001; Pasquinelli, A., Trends in Genetics, 18 (4), 171-173, 2002, and references in the foregoing two articles). MicroRNAs have been shown to block translation of target transcripts containing target sites in mammalian cells (Zeng, Y., et al., Molecular Cell, 9, 1-20, 2002).

Naturally occurring or artificial (i.e., designed by humans) mRNAs that bind within the 3′ UTR (or elsewhere in a target transcript) and inhibit translation may tolerate a larger number of mismatches in the mRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such mRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an RNAi-inducing agent, and any structure that generates such an RNA (i.e., serves as a precursor to the RNA) or serves as a template for transcription of such an RNA is useful in the practice of the present invention.

In the context of the present invention, RNAi-inducing agents such as siRNAs are useful both for therapeutic purposes, e.g., to modulate the expression of an EGR molecule or an EGR interacting molecule in a subject at risk of or suffering from schizophrenia and for various of the inventive methods for the identification of compounds for treatment of schizophrenia that modulate the activity or level of calcineurin. In particular, it is noted that RNAi has been shown to be effective in the mammalian brain and that vectors providing templates for transcription of shRNA molecules have been introduced into the brain and shown to downregulate local gene expression (Hommel, JD, et al., Nature Medicine, 9 (12) 1539-1544). It is further noted that numerous chemical modifications can be made to the siRNA duplex, or portions of one or both strands, and/or 3′ overhang(s) while not abolishing and frequently not significantly diminishing silencing activity (Dorsett, Y, and Tuschl, T., referenced above). Such modifications may, in general, enhance stability, cellular uptake, and/or intracellular efficacy of siRNA. The invention encompasses the use of siRNA or shRNA having any such modification, e.g., phosphorothioate, 2′-O methyl, 2′-O,4′-methylene nucleotides, etc., and others known in the art, e.g., from the antisense field.

The invention therefore provides a method of inhibiting expression of a gene encoding an EGR molecule or an EGR interacting molecule comprising the step of (i) providing a biological system in which expression of a gene encoding an EGR moelcule or an EGR interacting molecule is to be inhibited; and (ii) contacting the system with an RNAi-inducing agent targeted to a transcript encoding the calcineurin subunit or calcineurin interacting molecule. According to certain embodiments of the invention the subunit or molecule is encoded by a gene within or linked to a schizophrenia susceptibility locus, or within which a functional mutation causing or contributing to susceptibility or development of schizophrenia may exist. According to certain embodiments of the invention the biological system comprises a cell, and the contacting step comprises expressing the RNAi-inducing agent in the cell. According to certain embodiments of the invention the biological system comprises a subject, e.g., a mammalian subject such as a mouse or human, and the contacting step comprises administering the RNAi-inducing agent to the subject or comprises expressing the agent in the subject. According to certain embodiments of the invention the agent is expressed inducibly and/or in a cell-type or tissue specific manner.

The invention provides RNAi-inducing molecules (e.g., siRNA, shRNA, mRNA, or vectors providing templates for transcription of any of these) targeted to a transcript encoding any EGR molecule or EGR interacting molecule. In particular, the invention provides RNAi-inducing agents selectively or specifically targeted to a transcript encoding a polymorphic variant of such a transcript, wherein existence of the polymorphic variant in a subject is indicative of susceptibility to or presence of schizophrenia. The terms selectively or specifically targeted to, in this context, are intended to indicate that the RNAi-inducing agent causes greater reduction in expression of the variant than of other variants (i.e., variants whose existence in a subject is not indicative of susceptibility to or presence of schizophrenia). The transcript may encode, for example, any of the molecules listed in Table 1, or a polymorphic variant thereof. The RNAi-inducing agents, may be provided in the form of kits with additional components as appropriate.

B. Full and Partial Length Antisense RNA Transcripts. Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, Annals N Y Acad. Sci., 660: 70, 1992, Nellen, Trends Biochem. Sci., 18: 419, 1993; Baker and Monia, Biochim. Biophys. Acta, 1489: 3, 1999; Xu, et al., Gene Therapy, 7: 438, 2000; French and Gerdes, Curr. Opin. Microbiol., 3: 159, 2000; Terryn and Rouze, Trends Plant Sci., 5: 1360, 2000).

C. Antisense RNA and DNA Oligonucleotides.

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of an EGR molecule or EGR interacting polypeptide may be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Antisense oligonucleotides can be synthesized with a base sequence that is complementary to a portion of any RNA transcript in the cell. Antisense oligonucleotides may modulate gene expression through a variety of mechanisms including the modulation of RNA splicing, the modulation of RNA transport and the modulation of the translation of mRNA (Denhardt, 1992). Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) [Wagner, Nat. Medicine, 1: 1116, 1995; Varga, et al., Immun. Lett., 69: 217, 1999; Neilsen, Curr. Opin. Biotech., 10: 71, 1999; Woolf, Nucleic Acids Res., 18: 1763, 1990].

The invention provides a method of inhibiting expression of a gene encoding a calcineurin subunit or calcineurin interacting molecule comprising the step of (i) providing a biological system in which expression of a gene encoding an EGR molecule or an EGR interacting molecule is to be inhibited; and (ii) contacting the system with an antisense molecule that hybridizes to a transcript encoding the EGR molecule or EGR interacting molecule. According to certain embodiments of the invention the subunit or molecule is encoded by a gene within or linked to a schizophrenia susceptibility locus, or within which a functional mutation causing or contributing to susceptibility or development of schizophrenia may exist. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression may be inducible and/or tissue or cell type-specific. The antisense molecule may be an oligonucleotide or a longer nucleic acid molecule. The invention provides such antisense molecules.

D. Ribozymes. Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation [Cotten and Bimstiel, EMBO J. 8: 3861-3866, 1989; Usman, et al., Nucl. Acids Mol. Biol., 10: 243, 1996; Usman, et al., Curr. Opin. Struct. Biol., 1: 527, 1996; Sun, et al., Pharmacol. Rev., 52: 325, 2000]. See also e.g., Cotten and Bimstiel, “Ribozyme mediated destruction of RNA in vivo”, EMBO J. 8: 3861-3866, 1989.

The invention provides a method of inhibiting expression of a gene encoding a EGR molecule or EGR interacting molecule comprising the step of (i) providing a biological system in which expression of a gene encoding a calcineurin subunit or calcineurin interacting molecule is to be inhibited; and (ii) contacting the system with a ribozyme that hybridizes to a transcript encoding the calcineurin subunit or calcineurin interacting molecule and directs cleavage of the transcript. According to certain embodiments of the invention the subunit or molecule is encoded by a gene within or linked to a schizophrenia susceptibility locus, or within which a functional mutation causing or contributing to susceptibility or development of schizophrenia may exist. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression may be inducible and/or tissue or cell-type specific according to certain embodiments of the invention. The invention provides ribozymes designed to cleave transcripts encoding EGR molecules or EGR interacting molecules, or polymorphic variants thereof, as described above.

VIII. Pharmaceutical Compositions for Treatment of Schizophrenia or Schizophrenia Susceptibility.

Inventive compositions comprising compounds identified as described herein, or pharmaceutically acceptable salts thereof, may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include an active compound or salt thereof, or a related compound or analog, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. For delivery of nucleic acids or polypeptides for treatment or other purposes it may be desirable to use any of a variety of lipid and/or polymeric carriers and matrices. (See, e.g., patents and published PCT applications by Langer, et al. for discussion of polymer-based delivery strategies.) It may be desirable to employ a strategy such as that described in U.S. Pat. No. 6,316,003, relating to novel transport polypeptides which include HIV tat protein or one or more portions thereof, and which are covalently attached to cargo molecules to be delivered to cells.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition as described herein, can include a single treatment or, in many cases, can include a series of treatments.

Exemplary doses include milligram or microgram amounts of the inventive composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

Inventive pharmaceutical compositions can be included in a container, pack, or dispenser together with instructions for administration.

Any of the inventive compounds may be administered concurrently with an additional agent useful for treatment of schizophrenia. Many such agents are known in the art and include a wide variety of typical and atypical anti-psychotic agents. In addition, the compounds may be administered concurrently with compounds useful for ameliorating the side effects of anti-psychotic agents. See, for example, Hardman, J. G., et al., (eds.) Goodman & Gilman's The Pharmacological Basis of Therapeutics, 10^{th} edition, McGraw Hill, 2001, for discussion of numerous agents useful for the foregoing purposes. The concurrently administered compounds may be administered to the subject separately or may be formulated together.

IX. Methods and Reagents for Identification of Susceptibility Loci and Functional Mutations

The invention provides a systematic approach to identifying additional schizophrenia susceptibility loci, polymorphisms useful in diagnosis of schizophrenia or susceptibility to schizophrenia, and to identifying functional mutations that cause or contribute to schizophrenia. The invention provides a method of identifying a method of identifying a polymorphism useful in diagnosis of schizophrenia or susceptibility to schizophrenia comprising steps of (i) identifying one or more polymorphisms in or linked to a gene encoding a CN subunit or CN interacting protein; (ii) providing a set of samples including samples obtained from subjects affected with schizophrenia; and (iii) testing the samples for linkage or association of one or more variants of the polymorphism with schizophrenia. If linkage or assocation exists, the polymorphism is useful in diagnosis of schizophrenia or susceptibility to schizophrenia. Such polymorphisms may thus be located in or define a schizophrenia susceptibility locus. The set of samples may comprise samples obtained from one or more families affected with schizophrenia and may comprise both related and unrelated individuals.

The invention further provides a method of identifying a candidate functional mutation that causes or contributes to schizophrenia comprising steps of: (i) identifying a polymorphism in or linked to a gene encoding a CN subunit or CN interacting protein; (ii) determining that a polymorphic variant of the polymorphism is linked to or associated with susceptibilty to schizophrenia; (iii) sequencing the gene and optionally regulatory regions of the gene in a sample obtained from one or more subjects suffering from schizophrenia; (iv) comparing the sequence obtained with a normal or wild type sequence of the same gene; and (v) identifying the polymorphic variant as representing a mutation that causes or contributes to schizophrenia if the sequence obtained in step (iii) differs from the normal or wild type sequence.

The methods may further comprise analyzying expression of the gene in normal subjects and in subjects affected with schizophrenia, which includes examining the mRNA abundance, size, and tissue expression pattern, examining the abundance, size, tissue expression pattern and/or activity of the encoded protein, etc.

### EXAMPLES

### Example 1

### Locations of EGR genes, and Genes Encoding Molecules that Interact with EGR molecules, that are Coincident with Schizophrenia Susceptibility Loci

Materials and Methods

Gene location analysis. In the course of analyzing the association of the PPP3CC gene with schizophrenia, the human draft sequence (http://www.ncbi.nlm.nih.gov/cgi-bin/Entrez/hum_srch?chr-hum_chr.inf&query) was examined to identify candidate genes in the vicinity of PPP3CC that could contribute to the observed association signal. Among the neighboring genes is EGR3.

The scientific literature and/or the map viewer function/site of the human draft sequence (URL http://www.ncbi.nlm.nih.gov/cgi-bin/Entrez/hum_srch?chr-hum_chr.inf&query) was consulted using terms such as “EGR” to determine the precise chromosomal locations of other EGR molecules and of certain EGR interacting molecules. To identify additional EGR molecules the map viewer function/site was searched with query terms such as “EGR”, and the precise chromosomal locations of the genes were retrieved. Terms describing the chromosomal positions of the genes (e.g. chromosome 5 or 5q in the case of EGR1) were combined with the term “schizophrenia”, and the individual combinations were used to search the Entrez Pubmed data base (http://www.ncbi.nlm.nih.gov/Entrez/) to retrieve publications describing schizophrenia susceptibility loci present on those chromosomes. The chromosomal positions of the schizophrenia loci were compared with those of the genes to detect coincidence. For more detailed analysis, the Human Genome Browser (http://genome.ucsc.edu/cgi-bin/h Gatewav) or human Ensembi site (http://www.ensembl.org/Homo_{—}sapiens/) were used to compare the precise gene locations with locations of markers of maximal significance for a given region of susceptibility.

To identify additional EGR interacting molecules the map viewer function/site is further searched with query terms such as “EGR”, “NAB”, and the precise chromosomal locations of the genes, including EGR target genes (e.g., genes whose transcription is activated or repressed by EGR proteins) are retrieved. Terms describing the chromosomal positions of the genes are combined with the term “schizophrenia”, and the individual combinations are used to search the Entrez Pubmed data base (http://www.ncbi.nlm.nih.gov/Entrez/) to retrieve publications describing schizophrenia susceptibility loci present on those chromosomes. The chromosomal positions of the schizophrenia loci are compared with those of the genes to detect coincidence. For more detailed analysis, the Human Genome Browser (http://genome.ucsc.edu/cgi-bin/hgGateway) or human Ensembl site (http://www.ensembl.org/Homo_{—}sapiens/) are used to compare the precise gene locations with locations of markers of maximal significance for a given region of susceptibility.

Results

Locations ofgenes encoding EGR proteins and EGR binding proteins. To investigate whether mutations in EGR genes could contribute to schizophrenia etiology, chromosomal locations of the four known human EGR genes, EGR1, EGR2, EGR3, and EGR4, were compared with previously identified schizophrenia susceptibility loci. It was observed that the chromosomal locations of all four EGR genes coincide with previously mapped schizophrenia loci identified either by the inventors or others.

As described above, EGR3 is located at 8p21.3, within 150 kb of the PPP3CC gene (FIG. 1), for which we have observed an association with schizophrenia, and within a confirmed schizophrenia susceptibility locus (2-8).

EGR2 is located at 10q 21.3. A number of genetic studies performed by the inventors detected linkage between this chromosomal region with schizophrenia (Example 4).

EGR1 is located at 5q31.2 (FIG. 2), within another confirmed schizophrenia susceptibility locus (8-13).

EGR4 is located at 2p13.2 within a putative schizophrenia susceptibility locus at 2p13-14 (6,14-15).

Thus all four EGR gene family members are located within putative schizophrenia susceptibility loci identified by linkage studies.

### Example 2

### Sequence Analysis of Genes Encoding EGR Molecules or EGR Interacting Molecules in Schizophrenia Patients

Materials and Methods

Patient samples. Patient samples, e.g., United States and South African patient samples suitable for use in this study have been previously described (Liu H, Heath S C, Sobin C, Roos J L, Galke B L, Blundell M L, Lenane M, Robertson B, Wijsman E M, Rapoport J L, Gogos J A, Karayiorgou M. (2002) Proc Natl Acad Sci USA. March 19; 99 (6): 3717-22; Liu H, Abecasis G R, Heath S C, Knowles A, Demars S, Chen Y J, Roos J L, Rapoport J L, Gogos J A, Karayiorgou M. (2002) Proc Natl Acad Sci USA. December 24; 99 (26): 16859-64). Detailed information about one such sample, the adult schizophrenic (AS) sample, is provided in Sobin, C., Blundell, M. L., Conry, A., Weiller, F., Gavigan, C., Haiman, C. & Karayiorgou, M. (2001) Psychiatry Res. 101, 101-113. The South African sample is part of our ongoing collection of schizophrenia patients of Afrikaner origin and will be described in detail elsewhere (M. K., M. Torrington, C. S., B. R., S. C. H., M. L. B., H. Pretorius, S. Lay, J. A. G., and J. L. R., unpublished work). Probands in both samples meet lifetime criteria for Diagnostic and Statistical Manual of Mental Disorders, 4th Ed. (DSM-IV) (American Psychiatric Association. (1994) Diagnostic and Statistical Manual (Am. Psychiatric Assoc., Washington, D.C.)) schizophrenia or schizoaffective disorder. Participants were interviewed by specially trained clinicians by using the Diagnostic Interview for Genetic Studies (DIGS) (urnberger, J. I., et al. (1994) Arch. Gen. Psychiatry 51, 849-859). Detailed information about the COS sample is provided in Usiskin, S. I., et al. (1999) J. Am. Acad. Child Adolesc. Psychiatry 38, 1536-1543 and Nicolson, R., et al. (2000) Am. J. Psychiatry 157, 794-800. All COS probands met unmodified criteria for schizophrenia with onset of psychotic symptoms before their 13th birthday and mean age of onset of psychosis at 10.1 (+1.8 yr). National Institute of Mental Health (NIMH) samples are obtained from the NIMH Human Genetics Inititative dataset (http://zork.wustl.edu/nimh).

PCR/sequencing. PCR primers are designed to amplify genomic fragments spanning exon sequence including coding and non-coding exons, promoter sequence and some intron sequence. The human draft sequence available at the UCSC working human draft site, http://genome.ucsc.edu/ is used for all primer design. Each PCR primer pair consists of a forward and reverse primer designed to amplify a specific genomic fragment. Forward PCR primers contain 19-21 bp of homologous sequence fused on the 5′ end to an 18 bp forward universal sequencing tag: 5′-TGTAAAACGACGGCCAGT-3′ (SEQ ID NO: 4). Reverse PCR primers contain 19-21 bp of homologous sequence fused on the 5′ end to an 18 bp reverse universal sequencing tag: 5′-CAGGAAACAGCTATGACC-3′ (SEQ ID NO: 5). This allows all PCR fragments to be sequenced in both directions, using these two primers to prime all sequencing reactions. PCR reactions are performed using a programmable PCR tetrad machine (MJ Research, Cambridge Mass.). Certain of the reactions are performed using OptiPrime 10× PCR buffer 6 (Stratagene, La Jolla, Calif.). Specific sequences are amplified in a 25 ul reaction mixture containing 20 ng genomic DNA, each primer at 400 nM concentration, each dNTP at 200 uM concentration and 1.5 U Taq polymerase (Sigma Chemical Co., St. Louis, Mo.) in appropriate OptiPrime buffer conditions. PCR amplification conditions are as follows: an initial denaturation step at 94° C. for 5 min, followed by 34 amplification cycles: 45 sec at 94° C.; 60 sec at appropriate annealing temperature (usually 62.5° C.): 60 sec elongation at 72° C., followed by a final extension step at 72° C. for 7 min. GC rich fragments are amplified using Advantage GC genomic polymerase mix (Becton Dickenson, Palo Alto, Calif.) in reactions of 25 ul containing IM GC melt, and 400 nM primer concentration, according to the manufacturer's instructions. GC rich PCR amplification is as follows: an initial denaturation step at 95° C. for 1 min followed by 34 amplification cycles of: 94° C. for 30 sec, 68° C. for 3 min, followed by a final extension step at 68° C. for 7 min. PCR fragments are separated by 2% agarose gel electrophoresis, and purified using the Qiagen Minelute Gel extraction kit (Qiagen, Valencia, Calif.). All sequencing reactions are performed by ACGT Inc. (Northbrook, Ill.).

Sequence analysis. Sequence analysis is performed using DNAStar software. Patient sequences are compared with the human genome draft sequence, available at the UCSC website, http://genome.ucsc.edu/. Contigs including the patient sequences and the human draft sequence are constructed for each fragment, and polymorphisms are identified by comparison.

Genotyping. Polymorphisms (e.g., SNPs, microsatellite markers) used for genotyping are either identified by direct sequencing, or found in SNP databases including the UCSC working human draft site, (see web site having URL http://genome.ucsc.edu/), the Weizmann Institute Gene Cards site, (see web site having URL http://bioinfo.weizmann.ac.il/cards/index.html) which both link to the NCB1 SNP database, http://www.ncbi.nlm.nih.gov/SNP/; and the Celera database at http://www.celera.com/, the JSNP database (http://snp.ims.u-tokyo.ac.jp/), or elsewhere (e.g., from Applied Biosystems Assayon Demand. Insertion/deletion polymorphisms are typed by PCR of genomic DNA from individual subjects and identified by altered fragment size as assessed by agarose gel electrophoresis. Single nucleotide polymorphisms (SNPs) that alter restriction endonuclease sites are typed using PCR-Restriction Fragment Length Polymorphism Genotyping (Liu H, Heath S C, Sobin C, Roos J L, Galke B L, Blundell M L, Lenane M, Robertson B, Wijsman E M, Rapoport J L, Gogos J A, Karayiorgou M. (2002) Proc Natl Acad Sci USA. March 19; 99 (6): 3717-22; Liu H, Abecasis G R, Heath S C, Knowles A, Demars S, Chen Y J, Roos J L, Rapoport J L, Gogos J A, Karayiorgou M. (2002) Proc Natl Acad Sci USA. December 24; 99 (26): 16859-64). Briefly a fragment spanning the SNP is amplified by PCR from genomic DNA of individual subjects and 10 ul of the PCR product is digested with the relevant restriction enzyme in a 15 ul reaction according to the manufacturer's specifications (New England Biolabs, Beverly, Mass.). Digested products are subjected to 4% agarose gel epectrophoresis, visualized by ethidium bromide staining, and photographed with an Eagle Eye apparatus (Stratagene, La Jolla, Calif.). SNPs that do not alter restriction sites are genotyped using fluorescence polarization template-directed dye-terminator incorporation (FP-TDI) genotyping as described in publications referenced in Liu H, Heath S C, Sobin C, Roos J L, Galke B L, Blundell M L, Lenane M, Robertson B, Wijsman E M, Rapoport J L, Gogos J A, Karayiorgou M. (2002) Proc Natl Acad Sci USA. March 19; 99 (6): 3717-22; Liu H, Abecasis G R, Heath S C, Knowles A, Demars S, Chen Y J, Roos J L, Rapoport J L, Gogos J A, Karayiorgou M. (2002) Proc Natl Acad Sci USA. December 24; 99 (26): 16859-64 and other references mentioned herein).

Results

To identify potential functional polymorphisms in six genes that could contribute to schizophrenia susceptibility (genes encoding either EGR molecules or EGR interacting molecules), as well as polymorphisms that could be used for association studies, the sequence of coding and non-coding exons and some of the promoter region for these genes in genomic DNA isolated from a set of independent schizophrenia patients is determined.

The sequencing strategy consists of PCR amplification of fragments covering the regions to be sequenced, followed by sequencing of these fragments. PCR primers are designed to amplify fragments spanning exon sequence and some promoter sequence from patient genomic DNA, and ar tagged with universal sequencing primers so that all fragments can be sequenced in forward and reverse directions with the same two primers. Exon fragments containing at least 100 bp of flanking intron sequence on each side to cover splice donor and recipient sites and in some cases, putative branch sites, are sequenced. The obtained sequence is compared with the human draft sequence to identify polymorphisms.

### Example 3

### Association Studies of EGR Genes and Genes Encoding EGR Interacting Molecules and Identification of Association

Materials and Methods

Association analysis. Transmissions of single SNPs, as well as multiple SNP haplotypes, are analyzed by the transmission disequilibrium test using the Transmit program (Clayton, D., Am J Hum Genet. 1999 October; 65 (4): 1170-7). P values listed represent global significance levels calculated by the Transmit program, described at http://www-gene.cimr.cam.ac.uk/clayton/software/transmit.txt. P values are calculated from the global chi square values obtained from the Transmit program Clayton, D., Am J Hum Genet. 1999 October; 65 (4): 1170-7) TDT analysis. Alternatively or additionally, analysis is performed using extended transmission disequilibrium test (eTDT) and/or pedigree transmission disequilibrium test (PTDT) using the Transmit program. Similar programs or other means of similarly analyzing the data to identify transmission disequilibrium could also be used.

Results

To further investigate the involvement of genes encoding EGR molecules and EGR interacting molecules in schizophrenia pathogenesis, candidate genes (e.g, EGR1, EGR2, EGR3, EGR4, NAB 1, and/or NAB2) are systematically tested for association with disease in samples comprising a large number of affected families. Polymorphisms identified by direct sequencing (see above), supplemented with additional single nucleotide polymorphisms (SNPs) obtained from the NCB1, Celera, JSNP databases or from other sources are used. Suitable polymorphisms for use in the analysis for EGR1, EGR3, EGR4, and EGR2, respectively, are listed in Tables 2, 3, 4, and 5. The nature and frequency of the polymorphism, where known, is listed either in a separate column or in the column identifying the source. Name is an arbitrary identifier used for convenience.

TABLE 2
SNPs from the EGR1 genetic locus.

| Name/ | | Source: | Source: ABI | Source: |
|---|---|---|---|---|
| Position | NCBI | CELERA | Assayon Demand | JSNP |
| EGR1-J1 | rs3813321 | hCV2627461 | | IMS- |
| | | T/C 4/3 | | JST173438 |
| | | | | C:0.824, |
| | | | | T:0.176 |
| EGR1-J2 | | hCV2627459 | | |
| ˜1300 bp | | G/C 5/1 | | |
| EGR1-J3 | rs2294175 | hCV16186198 | | IMS- |
| ˜1650 bp | | | | JST047572 |
| EGR1-J4 | | hCV2627457 | C_2627457_10 | |
| ˜68 bp | | A/G 3/5 | | |
| EGR1-J5 | | hCV2627456 | | |
| | | G/T 2/4 | | |

TABLE 3
SNPs from the EGR3 genetic locus.

| Name/ | | Source: | Source: | Source: |
|---|---|---|---|---|
| Position | Variation | JSNP | NCBI | CELERA |
| EGR3-J1 | A/G | | rs1533307 | |
| 923 bp | | | | |
| EGR3-J2 | T/C | | rs1130425 | |
| 1079 bp | | | | |
| EGR3-J3 | G/A | | | hCV1601146 |
| 913 bp | | | | |
| EGR3-J4 | C/A | IMS-JST102118 | rs3750192 | |
| 2806 bp | | C:0.7378 | | |
| | | A:0.2622 | | |
| EGR3-J5 | G/T | | rs1049155 | hCV8793216 |

TABLE 4
SNPs from the EGR4 genetic locus.

| Name/ | Source: | Source: | Source: ABI | Source: |
|---|---|---|---|---|
| Position | NCBI | CELERA | AssayonDemand | JSNP |
| EGR4-J1 | rs1317734 | hCV197476 | | IMS- |
| ˜9000 bp | A = .757, | A/C = 3/4 | | JST067208 |
| | C = .243 | | | |
| EGR4-J2 | rs1522928 | hCV11940175 | | |
| ˜3700 bp | | A/G = 2/6 | | |
| EGR4-J3 | rs6747506 | hCV2072863 | | |
| ˜1100 bp | | A/T = 5/5 | | |
| EGR4-J4 | | hCV11539842 | | |
| ˜2360 bp | | A/G = 2/1 | | |
| EGR4-J5 | rs533641 | hCV1026678 | | |
| ˜440 bp | | | | |
| EGR4-J6 | rs2229294 | hCV3216608 | C_3216608_10 | |
| ˜1900 bp | C = .667, | T/C = 1/3 | | |
| | T = .333 | | | |
| EGR4-J7 | rs3813226 | hCV3216609 | | IMS- |
| ˜9700 bp | | T/C = 3/5 | | JST173330 |
| EGR4-J8 | rs6737049 | hCV3216613 | C_3216613_10 | |
| | | T/C = 3/3 | | |

First the genotypes for a number of schizophrenia triads (parents and affected proband) collected from the United States population (Liu H, Heath S C, Sobin C, Roos J L, Galke B L, Blundell M L, Lenane M, Robertson B, Wijsman E M, Rapoport J L, Gogos J A, Karayiorgou M. (2002) Proc Natl Acad Sci USA. March 19; 99 (6): 3717-22; Liu H, Abecasis G R, Heath S C, Knowles A, Demars S, Chen Y J, Roos J L, Rapoport J L, Gogos J A, Karayiorgou M. (2002) Proc Natl Acad Sci USA. December 24; 99 (26): 16859-64) with respect to all of the polymorphisms tested (referred to as SNPs hereafter for convenience). The transmission disequilibrium test (TDT) is then employed, using the Transmit program with a sliding window strategy to determine if observed transmission of any individual SNP or multiple (e.g., 2, 3, 4 and 5) SNP combinations deviate from the expected value in the sample.

Haplotypes having at least a certain frequency, e.g., 3%, are considered, and P values representing global significance are calculated from the global chi square values obtained from the TDT analysis. Genes for which significant deviation from expected transmission is observed for any of the individual or multiple SNP combinations are identified. SNPs or multiple SNP combinations in which P>0.05 but preferably <0.1 are identified as possibly representing a trend towards transmission disequilibrium and association with disease.

In general, obsrvation of statistically significant transmission disequilibrium for single SNP or multiple SNP haplotypes for SNP(s) in the sample collection for any particular gene provides evidence for association of that gene with schizophrenia. Such a result provides evidence that altered functional activity of the gene contributes to schizophrenia susceptibility and suggests that modulation (e.g., enhancing, inhibiting, or changing temporal and/or spatial activity pattern) could provide a therapeutic approach to treat or prevent schizophrenia. Such a result further provides evidence that screens for modulators of gene function are useful for identifying new therapies for schizophrenia and related disorders.

Failure to detect significant transmission disequilibrium for any particular genes in this study does not necessarily indicate a lack of association of these genes with schizophrenia. For example, different genes may be associated with schizophrenia in different populations, and it may be the case that studying such populations would reveal association. It may also be necessary to examine more SNPs for such genes, in order to ensure that the entire gene is represented since it is presently unclear whether all haplotype blocks are covered by the analysis described herein, as would be desirable. Furthermore, examining more families might reveal association since a sample of a limited number of families may not have enough power to detect a weaker but significant association. In addition, some genes may be prone to a high level of mutations or genomic instability, e.g., deletions. If this is the case, the mutations might happen so frequently, and are ongoing in the human population so that they will not be associated with a particular haplotype.

To further investigate the association of the any particular gene with schizophrenia, the genotypes with respect to any SNPs for which association is found in the initial test described above, is performed using an additional sample, preferably including both a collection of triads and larger pedigrees. It may be desirable to include samples collected from diverse geographic locations. The TDT, determined using the transmit program with a sliding window strategy, is again employed to determine if observed transmission of any individual SNP or multiple (2, 3, 4 and 5) SNP combinations deviate from the expected value in the combined sample. SNPs or multiple SNP combinations showing significant transmission disequilibrium (P<0.05) are identified. Highly significant transmission disequilibrium (e.g., P<0.001, or even lower P values) are particularly noted.

Examination of the transmissions of the individual haplotypes is performed to identify particulars multiple SNP haplotypes comprised of 2 or more SNPs that drive the observed transmission disequilibrium. Specifically haplotypes that are transmitted with greater than expected frequency with a high degree of statistical significance are identified. The identification of such haplotypes strongly suggests that variation in the relevant gene is associated with schizophrenia susceptibility and defines the particular haplotype as a risk haplotype for schizophrenia. Coding sequence mutations in this gene in any of the patients, which may be identified in the sequencing described above, are of special interest. Such SNPs may represent a functional mutation that contributes to disease susceptibility in some patients/families.

### Example 4

### Evidence for Association of EGR2 with Schizophrenia

The inventors performed a number of studies to investigate the possible association of EGR2 with schizophrenia. In particular, a genome wide linkage scan in a specific founder population sample was performed, and linkage of this chromosomal region with schizophrenia was detected. As indicated in the third column from the left in Table 5, Loki LOD scores of 1.77 and 2.18 were obtained. Loki (http://www.stat.washington.edu/thompson/Genepi/Loki.shtml) is a software package that analyses a quantitative trait observed on large pedigrees using Markov chain Monte Carlo multipoint linkage and segregation analysis. The trait may be determined by multiple loci.

This linkage was replicated in an independent sample of families collected in the United States. Analysis was performed using the GeneHunter program (http://www.cs.washington.edu/homes/pmork/final_project/GeneHunter.html), and nonparameteric LOD scores of 1.84 and 2 were obtained (Table 5, fourth column from left). EGR2 is located at position 63,916,359-63,920,718, adjacent to the observed peak marker for linkage in both studies. Analysis with microsatellite markers and single nucleotide polymorphisms in a third independent sample consisting of 210 triads provided nominally significant evidence for association with two microsatellite markers and two SNPs in the vicinity of the EGR2 gene (Table 5, rightmost column). Specifically, Haplotype Based Haplotype Relative Risk (HHRR) P-values of 0.001 and 0.004 were obtained for two microsatellite markers, and HHRR P-values of 0.05 were obtained for two SNPs (Table 5) (ns=not significant).

TABLE 5
Markers from the EGR4 genetic locus.

| | | | | HHRR |
|---|---|---|---|---|
| | POSITION ON | | | P-value |
| | CHROM. 10 | | GENE- | (In US |
| MARKER | (UCSC July | LOKI | HUNTER | triad |
| NAME | 2003 Assembly) | LOD | NPL | families) |
| Microsatellites | | | | |
| D10S196 | 51,386,871-51,587,052 | 1.77 | | 0.025 |
| D10S1220 | 51,922,985-52,123,547 | | 1.84 | |
| D10S1756 | 58,342,009-58,542,385 | | | |
| GATA151F09 | 63,343,786-63,544,041 | | | 0.001 |
| D10S1652 | 63,652,098-63,852,456 | 2.18 | | 0.024 |
| D10S1719 | 63,741,819-63,942,209 | | | 0.004 |
| D10S1225 | 63,999,602-64,199,891 | | 2 | 0.01 |
| D10S561 | 64,309,764-64,510,142 | | | 0.03 |
| SNPs | | | | |
| rs224120 | 63,785,363-63,795,363 | | | 0.05 |
| rs224131 | 63,804,697-63,814,697 | | | ns |
| rs224150 | 63,827,690-63,837,690 | | | ns |
| rs224029 | 63,858,902-63,868,902 | | | ns |
| rs1509966 | 63,892,210-63,902,210 | | | ns |
| rs1397030 | 63,938,109-63,948,109 | | | 0.05 |
| rs911610 | 63,959,645-63,969,645 | | | ns |
| rs1571923 | 63,981,816-63,991,816 | | | ns |
| rs1876919 | 64,019,557-64,029,557 | | | ns |
| rs2136614 | 64,040,889-64,050,889 | | | ns |

### Example 5

### Testing Candidate Compound in an Animal Model of Schizophrenia

Materials and Methods

This example describes tests that can be performed to assess the ability of a test compound (e.g., a compound identified according to screening methods described above) to alleviate phenotypes suggestive of schizophrenia, e.g., to restore parameters measured in the test to a more normal value. Additional tests, e.g., evaluation of nesting behavior, evaluation of latent inhibition, and variations on the tests described below, can also be used.

Animals and experimental design. The ability of a candidate compound that modulates EGR functional activity is tested to determine whether it alleviates phenotypes suggestive of schizophrenia in an animal model for schizophrenia. The compound may increase or decrease the ability of one or more EGR proteins to stimulate or repress transcription of one or more target genes, including synthetic reporter genes whose transcription is driven from an EGR DNA binding site. For example, the candidate compound may disrupt binding of EGR1, EGR2, and/or EGR3 with NAB1 and/or NAB2. Candidate compounds are identified as described elsewhere herein.

Suitable animal models include forebrain specific calcineurin knockout mice (CN mutants) as detailed in Zeng, H., Chattarji, S., Barbarosie, M., Rondi-Reig, L., Philpot, B. D., Miyakawa, T., Bear, M. F. and Tonegawa, S., Forebrain-specific calcineurin knockout selectively impairs bidirectional synaptic plasticity and working/episodic-like memory, Cell, 107 (2001) 617-29. Other suitable animal models can also be used (see above).

Compound adminstration. Groups of mice are administered varying doses of a candidate compound dissolved in a suitable solvent, or are administered solvent alone (control mice) by either intravenous injection, orally, or by injection into the brain. Testing is performed at various time points following either single or multiple doses of compound.

Motor function tests. Motor coordination and balance are tested with the rotarod test. The rotarod test is performed using an accelerating rotarod (UGO Basile Accelerating Rotarod) and consists of placing a mouse on a rotating drum (3 cm diameter) and measuring the time each animal is able to maintain its balance on the rod. The speed of the rotarod is accelerated from 4 to 40 rpm over a 5-min period.

Object exploration test. The test consists of 5 trials (10 min/trial). Mice are introduced into a box (40×40×30 cm) made of transparent white plexiglass and allowed to explore freely on the first day (trial 1) and the second day (trial 2) without objects. On the third day, they are placed in the box in the presence of two identical objects (object A; trial 3). Ten min after trial 3, one of the objects is replaced by a novel object (object B) and they are allowed to explore the box with the two different objects (object A and object B; trial 4). On the following day, object B is replaced by another novel object (Object A and object C; trial 5).

Behavior is monitored using a color CCD camera (Sony DXC-151A) connected to a Macintosh computer. Locomotor activity, and the time each animal spends around the objects, as well as the time spent in the center part of the field are recorded. Regions of interest (ROI) around the objects are defined as the circles with 8 cm diameter from the center of the object position. When the center of the mouse image is within the defined ROI for each object, the mouse is considered to be ‘around the object’. Analysis is performed automatically using Image OE software (see “Image analysis”). The recognition index (R1) is defined as (tB/(tA+tB))/100 as an index for memory on the objects.

Openfield test. Each subject is placed in the center of an open field apparatus (40×40×30 cm; Accuscan Instruments, Columbus, Ohio). The apparatus is cleaned with water after each trial. Total distance traveled (cm), vertical activity, time spent in the center and the number and duration of episodes and beam-brake counts for stereotyped behaviors are recorded. Data were collected over a 60 min-period.

Hot plate test. The hot plate test is used to evaluate sensitivity to a painful stimulus. Mice are placed on a 55.0 (±0.3)° C. hot plate (Columbus Instruments, Columbus, Ohio), and latency to the first hind-paw response is recorded. The hind-paw response is either a foot shake or a paw lick.

Light/dark transition test. The apparatus used for the light/dark transition test consists of a cage (21×42×25 cm) divided into two sections of equal size by a black partition containing a small opening (O'Hara & Co, Tokyo, Japan). One chamber is brightly illuminated, whereas the other chamber is dark. Mice are placed into the illuminated side and allowed to move freely between the two chambers for 10 min. The chambers are cleaned with water after each trial. The total number of transitions, time spent in the dark side, and distance traveled are recorded by Image LD4 software (see ‘Image analysis’).

Social interaction test. Two mice of identical genotype, which are housed in different cages, are placed into a box together (40×40×30 cm) and allowed to explore freely for 10 min. Social behavior is monitored using a CCD camera (Sony DXC-151A), which is connected to a Macintosh computer. Analysis is performed automatically using Image SI software (see ‘Image analysis’). The number of contacts, mean duration per contact, and total distance traveled are measured.

Latent inhibition test. On the training day (day 1), each mouse is placed in a shocking chamber (Coulbourn Instruments, Allentown, Pa.) (Box A). The mice are divided into two groups: preexposed group (P group) and non-preexposed group (NP group). The P group receives 40 tones (68 dB, 5 sec duration, 25 sec inter-stimulus interval), whereas the NP group receives no stimulus during an equivalent period. Immediately following the tone pre-exposure or the exposure to the chamber, tone-shock pairs consisting of a 5-sec white noise tone (CS) co-terminated with a 2-sec foot shock (US) at 0.40 mA are delivered to both groups with 25 sec inter-stimulus interval. Afterwards, mice remain in the chamber for 25 sec before being returned to the home cage. On day 2, the mice are placed back in Box A for 5 min for the measurement of freezing to the context. On day 3, the mice are put in a white plexiglass chamber (Box B) and, after 180 sec, a 180 sec tone is delivered to measure cued freezing.

Prepulse inhibition task. A startle reflex measurement system is used (MED Associates, St. Albans, Vt.). A test session is begun by placing a mouse in a plexiglas cylinder where it is left undisturbed for 5 min. The duration of white noise used as the startle stimulus is 40 msec for all trial types. The startle response is recorded for 160 msec (measuring the response every 1 msec) starting with the onset of the prepulse stimulus. The background noise level in each chamber is 70 dB. The peak startle amplitude recorded during the 160 msec sampling window is used as the dependent variable. A test session consists of 6 trial types (i.e. two types for startle stimulus only trials, and four types for prepulse inhibition trials). The intensity of startle stimulus is 100, 105, 110 or 120 dB. The prepulse sound is presented 100 msec before the startle stimulus, and its intensity was 74 or 78 dB. Four combinations of prepulse and startle stimuli are employed (74-110, 78-110, 74-120 and 78-120 for the first batch of subjects and the first test of the second batch of subjects; 74-100, 78-100, 74-105, and 78-105 for the second test of the second batch of animals). Six blocks of the 6 trial types (four trial types with the combinations of prepulse and startle stimulus and two startle stimulus only trials) are presented in pseudorandom order such that each trial type is presented once within a block. The average inter-trial interval is 15 sec (range: 10-20 sec).

Porsoltforced swim test. The apparatus consists of four glass beakers (15 cm height×10 cm diameter). The cylinders are separated from each other by a non-transparent panel to prevent mice from seeing each other. The cylinders are filled with water (23° C.), up to a height of 7.5 cm. Mice are placed into the cylinders and their behavior is recorded over a 10-min test period. Data acquisition and analysis are performed automatically, using Image PS software (see “Image analysis”). Distance traveled is measured by Image OF software (see “Image analysis”) using stored image files.

Quantification of nesting. Pictures of the nests are taken using a digital camera (Olympus, Melvile, N.Y.) and exported into a computer. The number of scattered particles of the nestlets is counted for each cage using the NIH Image program (see Image Analysis).

Image analysis. All applications used for the behavioral studies (Image SI, Image OE, Image LD4, Image PS, Image OF, and Image FZ) are run on a Macintosh computer. Applications are based on the public domain NIH Image program (developed by Wayne Rasband at the U.S. National Institute of Mental Health and available on the Internet at http://rsb.info.nih.gov/nih-image/) and are modified for each test by Tsuyoshi Miyakawa (available through O'Hara & Co., Tokyo, Japan).

Statistical analysis. Statistical analysis is conducted using StatView (SAS institute) or SAS (SAS institute). Data are analyzed by two tailed t-test, two-way ANOVA, or two-way repeated measures ANOVA. Values in tables and graphs are expressed as mean±SEM.

Results

To assess various aspects of activity and behavior, CN mutant mice (e.g., CN forebrain-specific knockout mice), or other animal models for schizophrenia (referred to in this example as “model mice”, are subjected to a battery of tests after administration of either candidate compound or solvent. These tests reflect aspects of activity and behavior that are altered in human subjects suffering from schizophrenia and other psychiatric disorders. Mice not receiving a candidate compound display a variety of abnormalities in behavior and/or activity indicative of phenotypes characteristic of schizophrenia and/or related disorders. The ability of a candidate compound to alleviate one or more of these abnormalities, e.g., to restore a parameter towards a more normal value is determined by comparing the relevant parameters in control (untreated) model mice and in model mice receiving various doses of the candidate compound. The effect of the compound on the relevant parameter is also assessed in wild type mice, e.g., wild type mice congenic to the model mice. Mice are also monitored for general signs of toxicity according to standard methods and their performance on tests designed to identify abnormalities in behavior and/or activity that are suggestive of schizophrenia.

Locomotor Activity, Stereotyped Behavior and Exploratory Behavior Towards Inanimate Objects.

Locomotor activity is measured using a variety of different tests. A hyperactivity phenotype as indicated, for example, by a greater total distance traveled during object exploration and social interaction tests, is generally characteristic of model mice. The number of vertical activities in the open field test and the counts and/or durations of stereotyped behaviors may also be significantly increased in the model mice relative to wild type. The ability of the compound to modify behavior and/or activity so as to restore locomotor activity and/or the number of episodes of stereotyped behavior of the model mice to more normal values is determined by counting the number of such episodes in model mice that either do or do not receive compound. The effect of the compound on this parameter in wild type mice is also assessed.

Time spent near the objects in model mice may be significantly longer than that of wild type mice. The ability of the compound to modify behavior and/or activity so as to restore the time spent near the objects to more normal values is determined by measuring this time model mice that either do or do not receive compound. The effect of the compound on this parameter in wild type mice is also assessed. The effect of the compound on recognition index parameter in model mice and in wild type mice is assessed.

The effect of the compound on “behavioral despair” as assessed by distance traveled and time spent in immobile posture in Porsolt forced swim test is determined comparing the foregoing parameters in model mice and in wild type mice that receive or do not receive the compound.

Decreased social interaction and increased anxiety-like behavior. Model mice may spend significantly less time in the central region of the open field apparatus which is generally considered to reflect increased anxiety (Crawley, J. N., What's Wrong With My Mouse? Behavioral Phenotyping of Transgenic and Knockout Mice, John Wiley & Sons, New York, 2000.) In the light/dark transition test, the number of transitions between the two compartments may be significantly decreased in model mice relative to wild type mice. The number of transitions is considered to be a better measure of anxiety than time spent in the lit compartment (Crawley, J. N., Exploratory behavior models of anxiety in mice, Neurosci Biobehav Rev, 9 (1985) 37-44). The ability of the compound to modify behavior and/or activity so as to restore the time course of locomotor activity to more normal values is determined by examining these parameters counting particles in model mice that either do or do not receive compound. The effect of the compound on these parameters in wild type mice is also assessed.

In social interaction tests, the number of social contacts of model mice may be lower than those of wild type mice, and/or the total and mean duration of contacts may be significantly shorter than those of wild type mice. The ability of the compound to modify behavior and/or activity so as to restore the number of social contacts to more normal values is determined in model mice that either do or do not receive compound. The effect of the compound on this parameter in wild type mice is also assessed.

Impaired prepulse inhibition. The percent prepulse inhibition, an index of sensorimotor gating, may be altered, e.g., it may be significantly lower in model mice than in wild type mice. The ability of the compound to modify behavior and/or activity so as to restore the percent prepulse inhibition to normal values is determined by comparing prepulse inhibition in model mice that either do or do not receive compound. The effect of the compound on this parameter in wild type mice is also assessed.

### REFERENCES

1. Gerber, D. J., Hall, D., Miyakawa, T., Demars, S., Gogos, J. A., Karayiorgou, M. & Tonegawa, S. (in press). Evidence for association of schizophrenia with genetic variation in the 8p21.3 gene, PPP3CC, encoding the calcineurin gamma subunit Proc Natl Acad Sci USA 100 (15): 8993-8998.
2. Pulver, A. E., Lasseter, V. K., Kasch, L., Wolyniec, P., Nestadt, G., Blouin, J. L., Kimberland, M., Babb, R., Vourlis, S., Chen, H. & et al. (1995) Am J Med Genet 60, 252-60.
3. Kendler, K. S., MacLean, C. J., O'Neill, F. A., Burke, J., Murphy, B., Duke, F., Shinkwin, R., Easter, S. M., Webb, B. T., Zhang, J., Walsh, D. & Straub, R. E. (1996) Am J Psychiatry 153, 1534-40.
4. Blouin, J. L., Dombroski, B. A., Nath, S. K., Lasseter, V. K., Wolyniec, P. S., Nestadt, G., Thomquist, M., Ullrich, G., McGrath, J., Kasch, L., Lamacz, M., Thomas, M. G., Gehrig, C., Radhakrishna, U., Snyder, S. E., Balk, K. G., Neufeld, K., Swartz, K. L., DeMarchi, N., Papadimitriou, G. N., Dikeos, D. G., Stefanis, C. N., Chakravarti, A., Childs, B., Pulver, A. E. & et al. (1998) Nat Genet 20, 70-3.
5. Gurling, H. M., Kalsi, G., Brynjolfson, J., Sigmundsson, T., Sherrington, R., Mankoo, B. S., Read, T., Murphy, P., Blayeri, E., McQuillin, A., Petursson, H. & Curtis, D. (2001) Am J Hum Genet 68, 661-73.
6. Straub, R. E., MacLean, C. J., Ma, Y., Webb, B. T., Myakishev, M. V., Harris-Kerr, C., Wommley, B., Sadek, H., Kadambi, B., O'Neill, F. A., Walsh, D. & Kendler, K. S. (2002) Mol Psychiatry 7, 542-59.
7. Badner, J. A. and Gershon, E. S. (2002) Mol Psychiatry 7, 405-411.
8. Lewis, C. M., Levinson, D. F., Wise, L. H., DeLisi, L. E., Straub, R. E., Hovatta, I., Williams, N. M., Schwab, S. G., Pulver, A. E., Faraone, S. V., Brzustowicz, L. M., Kaufmann, C. A., Garver, D. L., Gurling, H. M., Lindholm, E., Coon, H., Moises, H. W., Byerley, W., Shaw, S. H., Mesen, A., Sherrington, R., O'Neill, F. A., Walsh, D., Kendler, K. S., Ekelund, J., Paunio, T., Lonnqvist, J., Peltonen, L., et al. (2003) Genome Scan Meta-Analysis of Schizophrenia and Bipolar Disorder, Part II: Schizophrenia. Am J Hum Genet. July; 73 (1): 34-48.
9. Straub, R. E., MacLean, C. J., O'Neill, F. A., Walsh, D., Kendler, K. S. Support for a possible schizophrenia vulnerability locus in region 5q22-31 in Irish families. (1997) Mol Psychiatry. March; 2 (2): 148-55.
10. Schwab, S. G., Eckstein, G. N., Hallmayer, J., Lerer, B., Albus, M., Borrmann, M., Lichtermann, D., Ertl, M. A., Maier, W. & Wildenauer, D. B. (1997) Mol Psychiatry 2, 156-60.
11. Paunio, T., Ekelund, J., Varilo, T., Parker, A., Hovatta, I., Turunen, J. A., Rinard, K., Foti, A., Terwilliger, J. D., Juvonen, H., Suvisaari, J., Arajarvi, R., Suokas, J., Partonen, T., Lonnqvist, J., Meyer, J. & Peltonen, L. (2001) Hum Mol Genet 10, 3037-48.
12. Hovatta, I., Lichtermann, D., Juvonen, H., Suvisaari, J., Terwilliger, J. D., Arajarvi, R., Kokko-Sahin, M. L., Ekelund, J., Lonnqvist, J., Peltonen, L. Linkage analysis of putative schizophrenia gene candidate regions on chromosomes 3p, 5q, 6p, 8p, 20p and 22q in a population-based sampled Finnish family set. (1998) Mol Psychiatry. September; 3 (5): 452-7.
13. Crowe, R. R. & Vieland, V. (1999) Am J Med Genet 88, 229-32.
14. Coon, H., Myles-Worsley, M., Tiobech, J., Hoff, M., Rosenthal, J., Bennett, P., Reimherr, F., Wender, P., Dale, P., Polloi, A. & Byerley, W. (1998) Mol Psychiatry 3, 521-7.
15. Camp, N. J., Neuhausen, S. L., Tiobech, J., Polloi, A., Coon, H. & Myles-Worsley, M. (2001) Am J Hum Genet 69, 1278-89.
16. Mittelstadt, P. R. & Ashwell, J. D. (1998) Mol Cell Biol 18, 3744-51.
17. Yamagata, K., Kaufmann, W. E., Lanahan, A., Papapavlou, M., Barnes, C. A., Andreasson, K. I. & Worley, P. F. (1994) Learn Mem 1, 140-52.
18. Hippenmeyer, S., Shneider, N. A., Birchmeier, C., Burden, S. J., Jessell, T. M. & Arber, S. (2002) Neuron 36, 1035-49.
19. Miyakawa, T., Leiter, L. M., Gerber, D. J., Gainetdinov, R. R., Sotnikova, T. D., Zeng, H., Caron, M. G. & Tonegawa, S. Conditional calcineurin knockout mice exhibit multiple abnormal behaviors related to schizophrenia. Proc Natl Acad Sci USA, 100 (15): 8987-8992.
20. Tsai, G. & Coyle, J. T. (2002) Annu Rev Pharmacol Toxicol 42, 165-79.
21. Seeman, P. (1987) Synapse 1, 133-52.
22. Carlsson, A., Hansson, L. O., Waters, N. & Carlsson, M. L. (1997) Life Sci 61, 75-94.
23. Abi-Dargham, A., Rodenhiser, J., Printz, D., Zea-Ponce, Y., Gil, R., Kegeles, L. S., Weiss, R., Cooper, T. B., Mann, J. J., Van Heertum, R. L., et al. (2000) Proc. Natl. Acad. Sci. USA 97, 8104-8109
24. Laruelle, M., Abi-Dargham, A., van Dyck, C. H., Gil, R., D'Souza, C. D., Erdos, J., McCance, E., Rosenblatt, W., Fingado, C., Zoghbi, S. S., et al. (1996) Proc. Natl. Acad. Sci. USA 93, 9235-9240.
25. Breier, A., Su, T. P., Saunders, R., Carson, R. E., Kolachan, B. S., de Bartolomeis, A., Weinberger, D. R., Weisenfeld, N., Malhotra, A. K., Eckelman, W. C., et al. (1997) Proc. Natl. Acad. Sci. USA 94, 2569-2574.
26. Stefansson, H., Sigurdsson, E., Steinthorsdottir, V., Bjornsdottir, S., Sigrnundsson, T., Ghosh, S., Brynjolfsson, J., Gunnarsdottir, S., Ivarsson, O., Chou, T. T., Hjaltason, O., Birgisdottir, B., Jonsson, H., Gudnadottir, V. G., Gudmundsdottir, E., Bjornsson, A., Ingvarsson, B., Ingason, A., Sigfusson, S., Hardardottir, H., Harvey, R. P., Lai, D., Zhou, M., Brunner, D., Mutel, V., Gonzalo, A., Lemke, G., Sainz, J., Johannesson, G., Andresson, T., Gudbjartsson, D., Manolescu, A., Frigge, M. L., Gurney, M. E., Kong, A., Gulcher, J. R., Petursson, H. & Stefansson, K. (2002) Am J Hum Genet 71, 877-92.
27. O'Donovan K J, Tourtellotte W G, Millbrandt J, Baraban JM. (1999) The EGR family of transcription-regulatory factors: progress at the interface of molecular and systems neuroscience. Trends Neurosci. April; 22 (4): 167-73.
28. Madden S L, Rauscher FJ 3rd. (1993) Positive and negative regulation of transcription and cell growth mediated by the EGR family of zinc-finger gene products. Ann N Y Acad. Sci. June 11; 684: 75-84. Review.
29. Beckmann A M, Wilce P A. (1997) Egr transcription factors in the nervous system. Neurochem Int. October; 31 (4): 477-510; discussion 517-6. Review.
30. O'Donovan K J, Tourtellotte W G, Millbrandt J, Baraban JM. (1999) The EGR family of transcription-regulatory factors: progress at the interface of molecular and systems neuroscience. Trends Neurosci. April; 22 (4): 167-73.
31. Yoshino, M., Mizutani, T., Yamada, K., Tsuchiya, M., Minegishi, T., Yazawa, T., Kawata, H., Sekiguchi, T., Kajitani, T., Miyamoto, K. (2002) Early growth response gene-1 regulates the expression of the rat luteinizing hormone receptor gene. Biol Reprod. June; 66 (6): 1813-9.
32. Boss V, Talpade D J, Murphy TJ. (1996) Induction of NFAT-mediated transcription by Gq-coupled receptors in lymphoid and non-lymphoid cells. J Biol. Chem. 1996 May 3; 271 (18): 10429-32.
33. Boute N, Jockers R, Issad T. (2002). The use of resonance energy transfer in high-throughput screening: BRET versus FRET. Trends Pharmacol Sci. August; 23 (8): 351-4. Review.
34. Liu H, Heath S C, Sobin C, Roos J L, Galke B L, Blundell M L, Lenane M, Robertson B, Wijsman E M, Rapoport J L, Gogos J A, Karayiorgou M. (2002) Proc Natl Acad Sci U S A. March 19; 99 (6): 3717-22.
35. Liu H, Abecasis G R, Heath S C, Knowles A, Demars S, Chen Y J, Roos J L, Rapoport J L, Gogos J A, Karayiorgou M. (2002) Proc Natl Acad Sci USA. December 24; 99 (26): 16859-64.
36. Clayton, D., Am J Hum Genet. 1999 October; 65 (4): 1170-7.
37. Camp, N. J., Neuhausen, S. L., Tiobech, J., Polloi, A., Coon, H. and Myles-Worsley, M., Genomewide multipoint linkage analysis of seven extended Palauan pedigrees with schizophrenia, by a Markov-chain Monte Carlo method, Am J Hum Genet, 69 (2001) 1278-89.
38. Coon, H., Myles-Worsley, M., Tiobech, J., Hoff, M., Rosenthal, J., Bennett, P., Reimherr, F., Wender, P., Dale, P., Polloi, A. and Byerley, W., Evidence for a chromosome 2p13-14 schizophrenia susceptibility locus in families from Palau, Micronesia, Mol Psychiatry, 3 (1998) 521-7.
39. Gurling, H. M., Kalsi, G., Brynjolfson, J., Sigmundsson, T., Sherrington, R., Mankoo, B. S., Read, T., Murphy, P., Blayeri, E., McQuillin, A., Petursson, H. and Curtis, D., Genomewide genetic linkage analysis confirms the presence of susceptibility loci for schizophrenia, on chromosomes 1q32.2, 5q33.2, and 8p21-22 and provides support for linkage to schizophrenia, on chromosomes 11q23.3-24 and 20q12.1-11.23, Am J Hum Genet, 68 (2001) 661-73.
40. Kennedy, J. L., Basile, V. S. and Macciardi, F. M., Chromosome 4 Workshop Summary: Sixth World Congress on Psychiatric Genetics, Bonn, Germany, Oct. 6-10, 1998, Am J Med Genet, 88 (1999) 224-8.
41. Levinson, D. F., Mahtani, M. M., Nancarrow, D. J., Brown, D. M., Kruglyak, L., Kirby, A., Hayward, N. K., Crowe, R. R., Andreasen, N. C., Black, D. W., Silverman, J. M., Endicott, J., Sharpe, L., Mohs, R. C., Siever, L. J., Walters, M. K., Lennon, D. P., Jones, H. L., Nertney, D. A., Daly, M. J., Gladis, M. and Mowry, B. J., Genome scan of schizophrenia, Am JPsychiatry, 155 (1998) 741-50.
42. Bailer, U., Leisch, F., Meszaros, K., Lenzinger, E., Willinger, U., Strobl, R., Gebhardt, C., Gerhard, E., Fuchs, K., Sieghart, W., Kasper, S., Hornik, K. and Aschauer, H. N., Genome scan for susceptibility loci for schizophrenia, Neuropsychobiology, 42 (2000) 175-82
43. Karayiorgou, M. and Gogos, J. A., A turning point in schizophrenia genetics, Neuron, 19 (1997) 967-79.
44. Thaker, G. K. and Carpenter, W. T., Jr., Advances in schizophrenia, Nat Med, 7 (2001) 667-71.
45. Stober, G., Saar, K., Ruschendorf, F., Meyer, J., Nurnberg, G., Jatzke, S., Franzek, E., Reis, A., Lesch, K. P., Wienker, T. F. and Beckmann, H., Splitting schizophrenia: periodic catatonia-susceptibility locus on chromosome 15q15, Am J Hum Genet, 67 (2000) 1201-7.
46. Paunio, T., Ekelund, J., Varilo, T., Parker, A., Hovatta, I., Turunen, J. A., Rinard, K., Foti, A., Terwilliger, J. D., Juvonen, H., Suvisaari, J., Arajarvi, R., Suokas, J., Partonen, T., Lonnqvist, J., Meyer, J. and Peltonen, L., Genome-wide scan in a nationwide study sample of schizophrenia families in Finland reveals susceptibility loci on chromosomes 2q and 5q, Hum Mol Genet, 10 (2001) 3037-48.
47. Faraone, S. V., Matise, T., Svrakic, D., Pepple, J., Malaspina, D., Suarez, B., Hampe, C., Zambuto, C. T., Schmitt, K., Meyer, J., Markel, P., Lee, H., Harkavy Friedman, J., Kaufmann, C., Cloninger, C. R. and Tsuang, M. T., Genome scan of European-American schizophrenia pedigrees: results of the NIMH Genetics Initiative and Millennium Consortium, Am J Med Genet, 81 (1998) 290-5.
48. Craddock, N. and Lendon, C., Chromosome Workshop: chromosomes 11, 14, and 15, Am J Med Genet, 88 (1999) 244-54.
49. Chowdari, K. V., Xu, K., Zhang, F., Ma, C., Li, T., Xie, B. Y., Wood, J., Trucco, M., Tsoi, W. F., Saha, N., Rudert, W. A. and Nimgaonkar, V. L., Immune related genetic polymorphisms and schizophrenia among the Chinese, Hum Immunol, 62 (2001) 714-24.
50. Wright, P., Donaldson, P. T., Underhill, J. A., Choudhuri, K., Doherty, D. G. and Murray, R. M., Genetic association of the HLA DRB1 gene locus on chromosome 6p21.3 with schizophrenia, Am J Psychiatry, 153 (1996) 1530-3.
51. Brzustowicz, L. M., Hodgkinson, K. A., Chow, E. W., Honer, W. G. and Bassett, A. S., Location of a major susceptibility locus for familial schizophrenia on chromosome 1q21-q22, Science, 288 (2000) 678-82.
52. Freedman, R., Leonard, S., Olincy, A., Kaufmann, C. A., Malaspina, D., Cloninger, C. R., Svrakic, D., Faraone, S. V. and Tsuang, M. T., Evidence for the multigenic inheritance of schizophrenia, Am J Med Genet, 105 (2001) 794-800.
53. Schwab, S. G., Eckstein, G. N., Hallmayer, J., Lerer, B., Albus, M., Borrmann, M., Lichtermann, D., Ertl, M. A., Maier, W., and Wildenauer, D. B., Mol. Psychiatry. (1997) March; 2 (2): 156-60.
54. Crowe, R. R. and Vieland, V., Am J Med Genet (1999) June 18; 88 (3): 229-32.
55. Straub, R. E., MacLean, C. J., Ma, Y., Webb, B. T., Myakishev, M. V., Harris-Kerr, C., Wormley, B., Sadek, H., Kadambi, B., O'Neill, F. A., Walsh, D. and Kendler, K. S. Mol Psychiatry. 2002; 7 (6): 542-59.
56. Czemik, A. J., et al., Production ofphosphorylation state-specific antibodies. Methods Enzymol, 1991. 201: p. 264-83.
57. Boute N, Jockers R, Issad T. Trends Pharmacol Sci. 2002 August; 23 (8): 351-4.
58. Chumakov I, Blumenfeld M, Guerassimenko O, Cavarec L, Palicio M, Abderrahim H, Bougueleret L, Barry C, Tanaka H, La Rosa P, Puech A, Tahri N, Cohen-Akenine A, Delabrosse S, Lissarrague S, Picard F P, Maurice K, Essioux L, Millasseau P, Grel P, Debailleul V, Simon A M, Caterina D, Dufaure I, Malekzadeh K, Belova M, Luan J J, Bouillot M, Sambucy J L, Primas G, Saumier M, Boubkiri N, Martin-Saumier S, Nasroune M, Peixoto H, Delaye A, Pinchot V, Bastucci M, Guillou S, Chevillon M, Sainz-Fuertes R, Meguenni S, Aurich-Costa J, Cherif D, Gimalac A, Van Duijn C, Gauvreau D, Ouellette G, Fortier I, Raelson J, Sherbatich T, Riazanskaia N, Rogaev E, Raeymaekers P, Aerssens J, Konings F, Luyten W, Macciardi F, Sham P C, Straub R E, Weinberger D R, Cohen N, Cohen D, Ouelette G, Realson J., Proc Natl Acad Sci USA. 2002 Oct. 15; 99 (21): 13675-80.
59. Lewontin RC (1988) On measures of gametic disequilibrium. Genetics 120: 849-852.
60. Rothermundt, M., V. Arolt, and T. A. Bayer, Review of immunological and immunopathological findings in schizophrenia. Brain Behav Immun, 2001. 15 (4): p. 319-39.
61. Mohn, A. R., et al., Mice with reduced NMDA receptor expression display behaviors related to schizophrenia. Cell, 1999. 98 (4): p. 427-36.
62. Czemik, A. J., et al., Production of phosphorylation state-specific antibodies. Methods Enzymol, 1991. 201: p. 264-83.
63. Alberts, A. S., et al., Expression of a peptide inhibitor of protein phosphatase 1 increases phosphorylation and activity of CREB in NIH 3T3 fibroblasts. Mol Cell Biol, 1994. 14 (7): p. 4398-407.
64. Lefebvre, P., et al., Protein phosphatases 1 and 2A regulate the transcriptional and DNA binding activities of retinoic acid receptors. J Biol Chem, 1995. 270 (18): p. 10806-16.
65. Lin, X., et al., Inhibition of calcineurin phosphatase activity by a calcineurin B homologous protein. J Biol Chem, 1999. 274 (51): p. 36125-31.
66. Sun, L., et al., Cabin 1, a negative regulator for calcineurin signaling in T lymphocytes. Immunity, 1998. 8 (6): p. 703-11.
67. Lai, M. M., et al., Cain, a novel physiologic protein inhibitor of calcineurin. J Biol Chem, 1998. 273 (29): p. 18325-31.
68. Swimoff, A. H., et al., Nab1, a Corepressor of NGR1-A (Egr-1) Contains an Active Transcriptional Repression Domain, Mol. Cell. Biol., 1998. 18 (1): 512-524.
69. Sevetson, B R, et al., A Novel Activation Function for NAB Proteins in EGR-dependent Transcription of the Luteinizing Hormone b Gene, J. Biol. Chem., 2000. 275 (13): 9749-9757.
70. Svaren, J., et al., NAB2, a Corepressor of NGFIOA (Egr-1) and Krox20, Is Induced by Proliferative and Differentiative Stimuli, Mol. Cell. Biol., 1996. 16 (7): 3545-3553.
71. Ehrengruber, M. U., et al., Modulation of early growth resposne (EGR) transcription factor-dependent gene expression by using recombinant adenovirus, Gene, 2000. 258: 63-69.

1. A method for the diagnosis of schizophrenia or schizophrenia susceptibility comprising:
(i) providing a sample obtained from a subject to be tested for schizophrenia or schizophrenia susceptibility; and
(ii) detecting a polymorphic variant of a polymorphism in a coding or noncoding portion of a gene encoding an EGR molecule or encoding an EGR interacting molecule, or detecting a polymorphic variant of a polymorphism in a genomic region linked to such a gene, in the sample.

2. The method of claim 1, wherein the gene or a portion thereof is coincident with a schizophrenia susceptibility locus.

3. The method of claim 2, wherein the locus is a genetically identified locus.

4. The method of claim 1, wherein the polymorphism occurs in a coding or noncoding portion of a gene encoding an EGR molecule or encoding an EGR interacting molecule.

5. The method of claim 4, wherein the polymorphism occurs in a coding portion of the gene.

6. The method of claim 5, wherein the polymorphic variant results in an alteration in the amino acid sequence of the EGR protein or EGR interacting molecule encoded by the gene.

7. The method of claim 1, wherein the polymorphism occurs in a genomic region linked to a gene encoding an EGR molecule or encoding an EGR interacting molecule.

8. The method of claim 7, wherein the polymorphism is genetically linked to the gene.

9. The method of claim 1, wherein the gene encodes a polypeptide selected from the group consisting of: EGR1, EGR2, EGR3, EGR4, NAB1, and NAB2.

10. The method of claim 1, wherein the polymorphism is selected from the markers listed in any of Tables 2, 3, 4, and 5.

11. The method of claim 1, wherein the method is performed so as to detect, either in individually or in parallel, polymorphic variants of multiple polymorphisms in a coding or noncoding portion of one or more genes encoding an EGR molecule or encoding an EGR interacting molecule, or in a genomic region linked to such a gene, in the sample.

12. The method of claim 1, wherein the detecting step comprises:
contacting the sample with an oligonucleotide array, wherein the array comprises a plurality of oligonucleotides designed to specifically detect polymorphic variants of multiple polymorphisms in a coding or noncoding portion of one or more genes encoding an EGR molecule or encoding an EGR interacting molecule, or in a genomic region linked to such a gene, in the sample.

13. The method of claim 11 or 12, wherein the multiple polymorphisms comprise a risk haplotype for schizophrenia.

14. The method of claim 13, wherein at least one of the polymorphisms is selected from markers listed in any of Tables 2, 3, 4, or 5.

15. The method of claim 1, wherein the detecting step comprises:
contacting the sample with an oligonucleotide, wherein the oligonucleotide is designed to specifically detect or amplify a polymorphic variant of the polymorphism.

16. The method of claim 1, wherein the detecting step comprises:
contacting the sample with an oligonucleotide array, wherein the array comprises one or more oligonucleotides designed to specifically detect a polymorphic variant of the polymorphism.

17. The method of claim 1, further comprising the step of: determining that the subject is susceptible to or suffers from schizophrenia if the polymorphic variant is associated with an increased risk of schizophrenia.

18. A method for the diagnosis of schizophrenia or schizophrenia susceptibility comprising:
(i) providing a sample obtained from a subject to be tested for schizophrenia or schizophrenia susceptibility; and
(ii) detecting an alteration or variation in expression or activity of an EGR molecule or an EGR interacting molecule, in the sample, relative to the expression or activity of the EGR molecule or EGR interacting molecule that would be expected in a sample obtained from a normal subject.

19. The method of claim 18, wherein the alteration or variation comprises an increase or decrease in abundance of an mRNA that encodes the EGR molecule or EGR interacting molecule, or an increase or decrease in abundance of the EGR molecule or EGR interacting molecule.

20. The method of claim 18, wherein the EGR molecule or EGR interacting molecule is selected from the group consisting of: EGR1, EGR2, EGR3, EGR4, NAB 1, and NAB2.

21. The method of claim 18, wherein the alteration or variation results in an increase or decrease in transcription of an EGR target gene.

22. A method for the diagnosis of schizophrenia or schizophrenia susceptibility comprising:
(i) providing a sample obtained from a subject to be tested for schizophrenia or schizophrenia susceptibility; and
(ii) detecting an alteration or variation in an EGR molecule or EGR interacting molecule in the sample.

23. The method of claim 22, wherein the alteration or variation comprises an alteration or variation in the amino acid sequence, size, or tissue or subcellular distribution of the EGR molecule or EGR interacting molecule.

24. The method of claim 22, wherein the EGR molecule or EGR interacting molecule is selected from the group consisting of: EGR1, EGR2, EGR3, EGR4, NAB1, and NAB2.

25. The method of claim 22, wherein the detecting step comprises employing an antibody that specifically binds to the EGR molecule or EGR interacting molecule.

26. The method of claim 22, wherein the detecting step comprises employing an antibody that specifically binds to a variant of the EGR molecule or EGR interacting molecule, the presence of which variant is indicative of susceptibility or presence of schizophrenia.

27. A method for treating schizophrenia or susceptibility to schizophrenia comprising:
providing a subject at risk of or suffering from schizophrenia; and
administering a compound that modulates activity or abundance of an EGR molecule or EGR interacting molecule to the subject.

28. The method of claim 27, wherein the compound enhances activity or abundance of the EGR molecule or EGR interacting molecule.

29. The method of claim 27, wherein the compound reduces activity or abundance of the EGR molecule or EGR interacting molecule.

30. The method of claim 27, wherein the compound modulates activity of the EGR molecule or EGR interacting molecule.

31. The method of claim 30, wherein the compound enhances activity of the EGR molecule or EGR interacting molecule.

32. The method of claim 30, wherein the compound reduces activity of the EGR molecule or EGR interacting molecule.

33. The method of claim 27, wherein the compound modulates expression of the EGR molecule or EGR interacting molecule.

34. The method of claim 33, wherein the compound enhances expression of the EGR molecule or EGR interacting molecule.

35. The method of claim 33, wherein the compound reduces expression of the EGR molecule or EGR interacting molecule.

36. The method of claim 27, wherein the compound binds to the EGR molecule or EGR interacting molecule.

37. The method of claim 27, wherein the compound disrupts binding of EGR molecule or EGR interacting molecule to a second molecule.

38. The method of claim 37, wherein the compound disrupts binding of an EGR molecule and either NAB1 or NAB2.

39. The method of claim 27, wherein the EGR molecule or EGR interacting molecule is selected from the group consisting of: EGR1, EGR2, EGR3, EGR4, NAB1, and NAB2.

40. The method of claim 27, wherein the administering step comprises introducing a gene therapy vector into the subject.

41. The method of claim 40, wherein the gene therapy vector comprises a nucleic acid that encodes an EGR molecule or EGR interacting molecule or an expression product of a target gene of an EGR molecule or an EGR interacting molecule.

42. The method of any of claim 27, further comprising the step of: identifying the subject as at risk of or suffering from schizophrenia using the method of claim 1 or any other appropriate method.

43. A method of identifying a polymorphism useful in diagnosis of schizophrenia or susceptibility to schizophrenia comprising steps of:
identifying one or more polymorphisms in or linked to a gene encoding an EGR molecule or EGR interacting molecule;
providing a set of samples including samples obtained from subjects affected with schizophrenia;
testing the samples for linkage or association of one or more variants of the polymorphism with schizophrenia; and
identifying the polymorphism as useful in diagnosis of schizophrenia if linkage or association exists between one or more variants of the polymorphism and schizophrenia susceptibility.

44. A method of identifying a mutation that contributes to or causes schizophrenia or susceptibility to schizophrenia comprising steps of:
identifying a polymorphism in or linked to a gene encoding an EGR molecule or EGR interacting molecule;
determining that a polymorphic variant of the polymorphism is linked to or associated with susceptibilty to schizophrenia;
sequencing the gene and optionally regulatory regions of the gene in a sample obtained from one or more subjects suffering from schizophrenia;
comparing the sequence obtained with a normal or wild type sequence of the same gene;
identifying the polymorphic variant as representing a mutation that causes or contributes to schizophrenia if the sequence obtained in the sequencing step differs from the normal or wild type sequence.

45. A method for identifying a candidate compound for treatment of schizophrenia or susceptibility to schizophrenia comprising steps of:
providing a biological system comprising an EGR molecule and an EGR reporter;
contacting the biological system with a compound;
comparing the transcriptional response of the reporter in the presence of the compound with the response or expected response in the absence of the compound; and
identifying the compound as a candidate compound for treatment of schizophrenia or susceptibility to schizophrenia if the transcriptional response in the presence of the compound is different from the transcriptional response that occurs or would be expected in the absence of the compound.

46. The method of claim 45, wherein the biological system is a cell or population of cells.

47. The method of claim 45, wherein the biological system further comprises an NAB molecule.

48. A method of identifying a candidate compound for treatment of schizophrenia or susceptibility to schizophrenia comprising steps of:
providing a biological system comprising an EGR molecule and an endogenous EGR modulator;
contacting the biological system with the compound;
comparing extent or rate of binding of the EGR molecule and the endogenous EGR modulator in the presence of the compound with the extent or rate of binding that occurs or would be expected to occur in the absence of the compound; and
identifying the compound as a candidate compound for treatment of schizophrenia or susceptibility to schizophrenia if the extent or rate of binding of the EGR molecule and the endogenous EGR inhibitor in the presence of the compound is different from the extent or rate of binding that occurs or would be expected in the absence of the compound.

49. The method of claim 48, wherein the endogenous EGR modulator is an NAB protein.

50. The method of claim 48, wherein the comparing step comprises performing a two or three hybrid screen.

51. A method for identifying a candidate compound for treatment of schizophrenia or schizophrenia susceptibility comprising steps of:
providing a molecular structure of an EGR molecule;
identifying a structure that is expected to bind to the EGR molecule or to prevent binding of the EGR molecule to an EGR interacting molecule; and
selecting a compound having such a structure as a candidate compound for treatment of schizophrenia or schizophrenia susceptibility.

52. The method of any of claims 45, 48, or 51, further comprising the step of testing the compound in an animal model for schizophrenia or in human subjects at risk of or suffering from schizophrenia.

53. A method of identifying a compound for treatment of schizophrenia comprising steps of:
providing a subject or subjects;
administering a candidate compound to the subject or subjects, wherein the candidate compound modulates activity or abundance of an EGR molecule or EGR interacting molecule;
comparing severity or incidence of the phenotype in the subject or subjects to severity or incidence of the phenotype to that existing or expected to exist in a subject or subjects to which the candidate compound is not administered; and
identifying the candidate compound as a compound for the treatment of schizophrenia or schizophrenia susceptibility if severity or incidence of the phenotype in the subject or subjects is less than that existing or expected to exist in a subject or subjects to which the compound is not administered.

54. A method of identifying a compound for treatment of schizophrenia comprising steps of:
providing a subject or subjects at risk of or exhibiting one or more phenotypes suggestive of schizophrenia, wherein the subject or subjects have an alteration in at least one EGR molecule or EGR interacting molecule or in a gene encoding such a molecule;
administering a candidate compound to the subject or subjects;
comparing severity or incidence of the phenotype in the subject or subjects to severity or incidence of the phenotype to that existing or expected to exist in a subject or subjects to which the candidate compound is not administered; and
identifying the candidate compound as a compound for the treatment of schizophrenia or schizophrenia susceptibility if severity or incidence of the phenotype in the subject or subjects is less than that existing or expected to exist in a subject or subjects to which the compound is not administered.

55. The method of claim 54, wherein the alteration is an alteration in expression level or expression pattern.

56. The method of claim 54, wherein the alteration is an alteration in amino acid sequence.

57. The method of claim 54, wherein the subject or subjects exhibit a polymorphic variant of a gene encoding an EGR molecule or EGR interacting molecule, wherein presence of the variant is associated with one or more phenotypes suggestive of schizophrenia.

58. The method of claim 54, wherein the subject or subjects are mice.

59. The method of claim 54, wherein the subject or subjects are humans.

60. The method of claim 54, wherein the subject or subjects are genetically engineered.

61. The method of claim 54, wherein the subject or subjects have an alteration in at least two molecules selected from the group consisting of EGR molecules and EGR interacting molecules.

62. The method of claim 54, wherein the subject or subjects are deficient in expression of at least one EGR molecule or EGR interacting molecule.

63. A compound identified according to the method of any of claims 45, 48, 51, 53, or 54, or a derivative thereof, wherein the derivative optionally displays enhanced bioavailability, enhanced ability to cross the blood-brain barrier, or an improved safety profile.

64. A pharmaceutical composition comprising:
the compound of claim 63; and
a pharmaceutically acceptable carrier.

65. A method of treating schizophrenia or susceptibility to schizophrenia comprising steps of:
providing a subject at risk of or suffering from schizophrenia; and
administering any of the pharmaceutical compositions of claim 64 to the subject either alone or concurrently with a second compound for treatment of schizophrenia or schizophrenia susceptibility or a compound that reduces side effects of such a compound.

66. The method of claim 65, further comprising the step of identifying the subject as at risk of or suffering from schizophrenia according to the method of claim 1.

67. An oligonucleotide designed to specifically detect or amplify a naturally occurring polymorphic variant of a polymorphism in a coding or noncoding portion of a gene encoding an EGR molecule or EGR interacting molecule, or a polymorphic variant of a polymorphism in a genomic region linked to such a gene, wherein the gene or a portion thereof is coincident with a schizophrenia susceptibility locus.

68. The oligonucleotide of claim 67, wherein the gene is selected from the group consisting of: EGR1, EGR2, EGR3, EGR4, NAB 1, and NAB2.

69. The oligonucleotide of claim 68, wherein the polymorphism is the selected from the markers listed in any of Tables 2, 3, 4, and 5.

70. An oligonucleotide designed to specifically detect or amplify a naturally occurring nucleic acid region comprising a polymorphic site in a coding or noncoding portion of a gene encoding an EGR molecule or EGR interacting molecule, or a polymorphic variant of a polymorphism in a genomic region linked to such a gene, wherein the gene or a portion thereof is coincident with a schizophrenia susceptibility locus.

71. The oligonucleotide of claim 70, wherein the schizophrenia susceptibility locus is genetically identified.

72. The oligonucleotide of claim 70, wherein the gene is selected from the group consisting of: EGR1, EGR2, EGR3, EGR4, NAB1, and NAB2.

73. The oligonucleotide of claim 72, wherein the polymorphism is the selected from the markers listed in any of Tables 2, 3, 4, and 5.

74. A pair of oligonucleotides as set forth in claim 67 or 70, wherein the oligonucleotides hybridize to opposite DNA strands on either side of the polymorphic site.

75. A kit comprising the oligonucleotide of claim 67 or 70 and one or more items selected from the group consisting of: packaging and instructions for use, a buffer, nucleotides, a polymerase, an enzyme, a positive control sample, a negative control sample, and a negative control primer or probe.

76. An oligonucleotide array comprising a plurality of oligonucleotides as set forth in claim 67 or 70.

77. The oligonucleotide array of claim 76, wherein the oligonucleotides detect polymorphic variants at a plurality of different polymorphic sites.

78. A kit comprising the oligonucleotide array of claim 76 and one or more items selected from the group consisting of: packaging and instructions for use, a buffer, nucleotides, a polymerase, an enzyme, a positive control sample, a negative control sample, and a negative control primer or probe.

79. A primer that terminates at the nucleotide position immediately adjacent to a naturally occurring polymorphic site on the 3′ side and extends at least 8 and less than 100 nucleotides in the 5′ direction from this site, wherein the polymorphic site is the site of a polymorphism in a coding or noncoding portion of a gene encoding an EGR molecule or EGR interacting molecule or is the site of a polymorphism in a genomic region linked to such a gene.

80. The primer of claim 79, wherein the gene or a portion thereof is coincident with a schizophrenia susceptibility locus.

81. The primer of claim 80, wherein the schizophrenia susceptiblity locus is genetically identified.

82. The primer of claim 80, wherein the gene is selected from the group consisting of: EGR1, EGR2, EGR3, EGR4, NAB1, and NAB2.

83. The primer of claim 80, wherein the polymorphism is the selected from the markers listed in any of Tables 2, 3, 4, and 5.

84. A pair of primers as set forth in claim 79, wherein the primers hybridize to opposite DNA strands adjacent to the location of the polymorphic site.

85. A kit comprising the primer of claim 79 and one or more items selected from the group consising of: packaging and instructions for use, a buffer, nucleotides, a polymerase, an enzyme, a positive control sample, a negative control sample, and a negative control primer or probe.

86. An siRNA or shRNA molecule targeted to a transcript encoding an EGR molecule or EGR interacting molecule.

87. The siRNA or shRNA molecule of claim 86, wherein the EGR molecule or EGR interacting molecule is encoded by a gene that is coincident with a schizophrenia susceptibility locus.

88. The siRNA or shRNA molecule of claim 87, wherein the schizophrenia susceptibility locus is genetically identified.

89. The siRNA or shRNA molecule of claim 86, wherein the molecule is selectively or specifically targeted to a transcript encoding a polymorphic variant of such a transcript, wherein existence of the polymorphic variant in a subject is indicative of susceptibility to or presence of schizophrenia.

90. The siRNA or shRNA molecule of claim 86, wherein the transcript encodes a molecule selected from the group consisting of: EGR1, EGR2, EGR3, EGR4, NAB1, and NAB2.

91. A kit comprising the siRNA or shRNA molecule of claim 86 and one or more items selected from the group consisting of: packaging and instructions for use, a buffer, a positive control sample, and a negative control siRNA or shRNA.

92. An antibody that specifically binds to a variant of an EGR molecule or EGR interacting molecule, wherein the calcineurin subunit or calcineurin interacting molecule is encoded by a gene comprising a polymorphic variant, wherein existence of the polymorphic variant in a subject is indicative of susceptibility to or presence of schizophrenia.

93. The antibody of claim 92, wherein the calcineurin subunit or calcineurin interacting molecule is selected from the group consisting of: EGR1, EGR2, EGR3, EGR4, NAB 1, and NAB2.

94. A kit comprising the antibody of claim 92 and one or more items selected from the group consisting of: packaging and instructions for use, a buffer, a substrate, a secondary antibody, an enzyme, a positive control sample, a negative control sample, and a negative control antibody.

95. A database comprising a list of polymorphic sequences stored on a computer-readable medium, wherein the polymorphic sequences occur in a coding or noncoding portion of a gene encoding an EGR molecule or EGR interacting molecule, or in a genomic region linked to such a gene, and wherein the list is largely or entirely limited to polymorphisms have been identified as useful in performing genetic diagnosis of schizophrenia or susceptibility to schizophrenia, or for performing genetic studies of schizophrenia or susceptibility to schizophrenia.
